(19) United States
(12) Patent Application Publication
(10) Pub. No.: US 2008/0283122 A1
(43) Pub. Date: Nov. 20, 2008

(54) Beta-Substituted Porphyrins

(76) Inventors: Wayne Mason Campbell, Palmerston North (NZ); David Leslie Officer, New South Wales (AU); Michael Graetzel, Vaud (CH); Md. Khaja Nazeeruddin, Vaud (CH)

Correspondence Address: BALDWINS, LEVEL 14, BALDWIN CENTER, 342 LAMBTON QUAY, WELLINGTON, NZ

(21) Appl. No.: 11/576,690

(22) PCT Filed: Oct. 10, 2005
(86) PCT No.: PCT/NZ05/00259

§ 371 Date: Apr. 22, 2008

(30) Foreign Application Priority Data
Oct. 8, 2004 (NZ) 535858

(51) Int. Cl. H01L 31/00; C07D 225/00

(52) U.S. Cl. 136/265; 540/465

(57) ABSTRACT

The invention relates to ÿ-substituted porphyrins, methods for their synthesis, and their use in the preparation of photoelectric materials. In particular, the invention relates to solid state photoelectric devices, including solar cells and photodetectors, incorporating these photoelectric materials, with improved photon-to-current conversion efficiencies.

### FIELD OF INVENTION

The invention relates to β-substituted porphyrins, methods for their synthesis, and their use in the preparation of photoelectric materials. In particular, the invention relates to solid state photoelectric devices, including solar cells and photodetectors, incorporating these photoelectric materials, with improved photon-to-current conversion efficiencies.

### BACKGROUND

Photoelectronic devices are devices that function on the basis of the photoelectric effect, namely, the absorption of photon (light) energy by electrons, leading to their release from a surface or otherwise allowing conduction. The efficiency of such devices is measured in terms of photon-to-current conversion.

Photoelectronic devices include photoelectro-chemical cells (PECs), more commonly referred to as solar cells, such as the Grätzel Cell (Hagfeldt, A.; Grätzel, M. Acc. Chem. Res., 2000, 33, 269-277) and solid state heterojunction devices (Wienke, J.; Schaafsma, T. J.; Goossens, A. J Phys. Chem., B, 1999, 103, 2702-2708).

Photoelectric materials used in the manufacture of these devices include semiconductors. In these semiconductor-based devices photon energy is absorbed and excited electrons are injected into the conduction band of the semiconductor. Zinc oxide (ZnO), titanium dioxide (TiO₂) and tin dioxide (SnO₂) are wide-band-gap (≧3.0 eV) semiconductors. These semiconductors absorb photon energy with wavelengths ≦413 nm.

Accordingly, these semiconductors are nearly transparent to the major part of the solar light spectrum. Methods of sensitising these semiconductors to increase their absorbance in the visible part of the light spectrum have been sought.

To increase the efficiency of absorption of photon energy from solar light, the semiconductors are coated with a thin layer of sensitising dye (chromophore). If the oxidative energy level of the excited state of the dye molecule is favourable (i.e. more negative) with respect to the conduction band energy level of the semiconductor, then there will be electron transfer and injection of an excited electron into the conduction band of the semiconductor.

Titanium dioxide is a preferred substrate for the preparation of dye-sensitised semiconductors (DSSCs). It is a chemically inert, non-toxic and biocompatible semiconductor readily available in high purity. It therefore represents an economical and ecologically safe semiconductor for use in the preparation of photoelectric materials.

Thin films of TiO₂ are prepared by many different physical and chemical techniques such as thermal oxidation, sputtering and chemical vapour deposition. Transparent mesoporous nanocrystalline films of TiO₂ with large surface area may be prepared, for example by depositing nanosized colloidal TiO₂ particles on a support.

Coating mesoporous nanocrystalline films of TiO₂ with a thin layer of sensitising dye has provided DSSCs with absorbance in the visible part of the solar light spectrum and improved solar energy conversion efficiency. To date, the most successful DSSCs are ruthenium-polypyridyl based dyes adsorbed on nanocrystalline films of TiO₂ (M. K. Nazeeruddin, P. Pechy, T. Renouard, S. M. Zakeeruddin, R. Humphry-Baker, P. Comte, P. Liska, L. Cevey, E. Costa, V. Shklover, L. Spiccia, G. B. Deacon, C. A. Bignozzi, M. Grätzel, J. Am. Chem. Soc. 123 (2000) 1613.) Recently, purely organic-based (coumarin bithiophene linked) DSSCs have been reported that provide energy conversion efficiencies of 7.7% (K. Hara, M. Kurashige, Y. Dan-oh, C. Kasada, A. Shinpo, S. Suga, K. Sayama, H. Arakawa, New J. Chem 27 (2003) 783).

These DSSCs absorb across the visible light spectrum. The dyes desirably bind strongly to the TiO₂ surface and have a suitably high redox potential for regeneration following excitation. However, ruthenium-based dyes are likely to become increasingly more expensive as the demand for ruthenium raw materials increases. Alternatives to ruthenium-polypyridyl complexes for use as sensitising dyes have therefore been sought.

The use of porphyrins as sensitising dyes is particularly attractive given their primary role in photosynthesis and the relative ease with which a variety of covalent or noncovalent porphyrin arrays (“molecular antennae”) can be constructed. The attachment of a large porphyrin array to a nanocrystalline semiconductor surface provides a way to dramatically increase the surface dye concentration and therefore, the light energy conversion efficiency of the device.

Various porphyrins have been used for the photosensitisation of wide-band-gap semiconductors like NiO, ZnO and TiO₂, the most common being the free-base and zinc derivatives of the meso-benzoic acid substituted porphyrin tetrakis(4-carboxyphenyl).

These porphyrins exhibit long-lived (>1 ns) π* singlet excited states and only weak single/triplet mixing. They have an appropriate LUMO level that resides above the conduction band of the TiO₂ and a HOMO level that lies below the redox couple in the electrolyte solution. This is required for charge separation at the semiconductor-dye-electrolyte surface.

Investigations into the efficiency of DSSCs sensitised with porphyrins bound to a surface through the β-pyrollic position of the porphyrin macrocycle are rare. The use of natural porphyrins, and zinc and antimony metallo-uroporphyrins as photosensisisers has been investigated. Monochromatic photon-to-current conversion efficiency values of 4.5% at 540 nm for a zinc-uroporhyrin have been obtained.

Grätzel and his group have also studied a number of natural metallochlorophyll derivatives and related natural metallo-meso-porphyrins for the photosensitisation of TiO₂ solar cells. These cells showed efficient sensitisation of TiO₂, with near unity quantum efficiency of charge injection for Soret peak illumination of Cu-meso-porphyrin. However, the solar light energy conversion efficiency value of the cell was 2.6%.

In these studies it was concluded that free carboxyl groups are important for adsorption, however, conjugation of the carboxyl groups to the π electron system of the chromophore is not necessary for efficient electron transfer with a linker of the length used. The study also revealed a strong dependence on the type of co-adsorbates and type of solvent containing the redox electrolyte.

Using ruthenium sensitizers and a nitrile-based electrolyte, the efficiency of nanocrystalline TiO₂ solar cells has reached more than 11% at AM 1.5 sunlight. (M. Grätzel, J. Photochem. Photobiol., C, 4, 145 (2003)). However, in addition to the use of ruthenium sensitisers another long-term limitation of these PECs is the use of liquid electrolytes and a need to find a sealing method that can resist the organic solvents containing the electrolyte.

A solution to the problem is the replacement of the liquid electrolyte by a solid hole-conducting electrolyte. For this reason, gel-based electrolytes (P. Wang, Q. Dai, S. M. Zakeeruddin, M. Forsyth, D. R. MacFarlane, M. Grätzel, J. Am. Chem. Soc. 2004, 126, 13 590), polymers (W. Kubo, K. Murakoshi, T. Kitamura, S. Yoshida, M. Haruki, K. Hanabusa, H. Shirai, Y. Wada, S. Yanagida, J. Phys. Chem. B 2001, 105, 12 809) and p type semiconductors have been extensively studied (B. O'Regan, F. Lenzmann, R. Muis, J. Wienke, Chem. of Materials 2002, 14, 5023).

Bach et al. (U. Bach, D. Lupo, P. Compte, J. E. Moser, F. Weissgrtel, J. Salbeck, H. Spreitzer, M. Grätzel, Nature 1998, 395, 583) have demonstrated that the liquid electrolyte can be replaced by an amorphous organic hole-transport material 2,2′,7,7′-tetrakis(N,N-di-p-methoxyphenylamine)-9,9′-spirobi-fluorene (spiro-MeOTAD) creating a solid p-type semiconductor/TiO₂ heterojunction. This hole-conducting material allows the regeneration of the sensitizers after electron injection due to its efficient hole-transport properties. However, the overall cell conversion efficiency using the ruthenium dye [Ru—(H₂dcbpy)₂(NCS)₂] was significantly lower than the value of 11% observed for the corresponding liquid-junction cell (J. Kruger, R. Plass, L. Cevey, M. Piccirelli, M. Grätzel, Appl. Phys. Lett. 2001, 79, 2085).

Accordingly there remains a need for porphyrin dyes as an alternative to ruthenium dyes that can be used in the preparation if DSSCs, especially DSSCs that provide a less significant loss of conversion efficiency when used in conjunction with a gelled or solid electrolyte or hole-transport material. As nanocrystallined TiO₂ based DSSCs are translucent to the eye, practical applications such as photovoltaic windows would then be rendered possible.

It is an object of this invention to provide β-substituted porphyrin dyes for use in the preparation of DSSCs.

It is a further object of this invention to provide DSSCs for use in the manufacture of solid state photoelectric devices with improved conversion efficiencies.

These objects of the invention are to be read disjunctively with the object to at least provide a useful choice.

### STATEMENTS OF INVENTION

In a first aspect the invention provides a photoelectric device incorporating a dye-sensitised semiconductor where the bound dye has the structure:

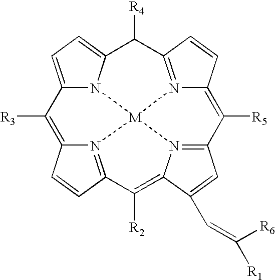

and where:
R₁ is selected from the group consisting of: carboxylic acids, phosphonic acids, sulfonic acids, or salts thereof;
R₂, R₃, R and R₅ are independently selected from the group consisting of: H, substituted or unsubstituted alkyl, substituted or unsubstituted aryl, and substituted or unsubstituted alkyl aryl;
R₆ is selected from the group consisting of: H, CN or —COOH; and
M is absent (and the porphin exists in the free base, protonated diacid, or dianion form) or is selected from the group consisting of: Cu, Ni or Zn.

Preferably the porphin of the dye exists in the metallated form. More preferably the porphin of the dye is metallated with Zn.

Preferably the semiconductor is selected from the group consisting of: zinc oxide (ZnO), titanium dioxide (TiO₂) and tin dioxide (SnO₂). More preferably titanium dioxide (TiO₂).

Preferably the semiconductor is in a mesoporous nanocrystalline form.

Preferably the photoelectric device is a solid state device including a gelled or solid electrolyte or hole transport material.

Preferably R₁ is a carboxylic acid selected from the group consisting of: cyanoacetatic acids, malonatic acids, or salts thereof.

Preferably R₂, R₃, R₄ and R₅ are independently selected from the group consisting of: tert-butyl, phenyl, methylphenyl, methoxyphenyl, ethylphenyl, dimethylphenyl (xylyl), tert-butylphenyl, octylphenyl, di-tert-butylphenyl, and methoxyphenyl.

Preferably R₆ is selected from the group consisting of: H or CN.

Preferably the semiconductor includes a surface coating of a non-acceptor. More preferably the non-acceptor is selected from the group consisting of: 4-tert-butylpyridine and Nb2O₅. Preferably the electrolyte or hole transport material comprises 2,2′,7,7′-tetrakis(N,N-di-p-methoxyphenyl-amine)9,9′-spirobifluorene.

Preferably the electrolyte or hole transport material further comprises tris-(4-bromophenyl)-ammoniumylhexachloroantimonate.

Preferably the electrolyte or hole transport material comprises lithium triflate and tert-butylpyridine.

Preferably the photoelectric device is a photoelectro-chemical cell. More preferably the photoelectric device is a photoelectro-chemical cell with an overall conversion efficiency of at least 2.5%. Most preferably the photoelectric device is a photoelectro-chemical cell with an overall conversion efficiency of at least 3.0%.

In a first embodiment the dye is a cyanoacetic acid and selected from the group consisting of:

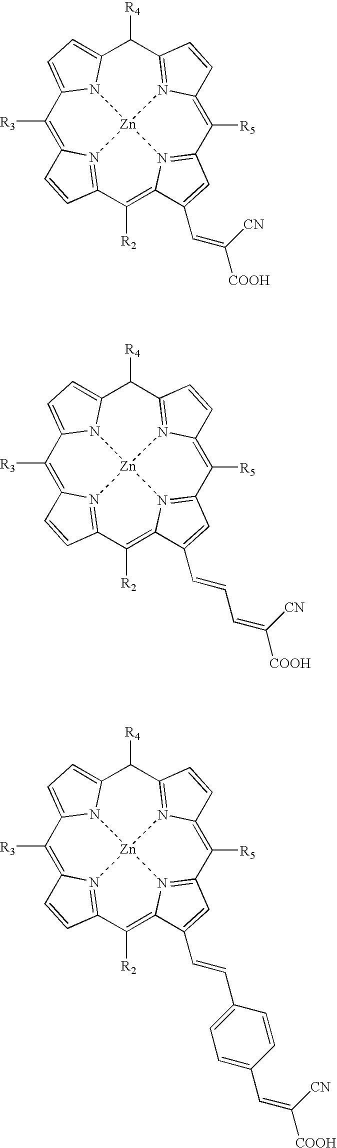

where R₂, R₃, R₄ and R₅ are tert-butyl, phenyl, methylphenyl, ethylphenyl, dimethylphenyl (xylyl), tert-butylphenyl, octylphenyl, di-tert-butylphenyl, or methoxyphenyl.

In a second embodiment the dye is a malonic acid and selected from the group consisting of:

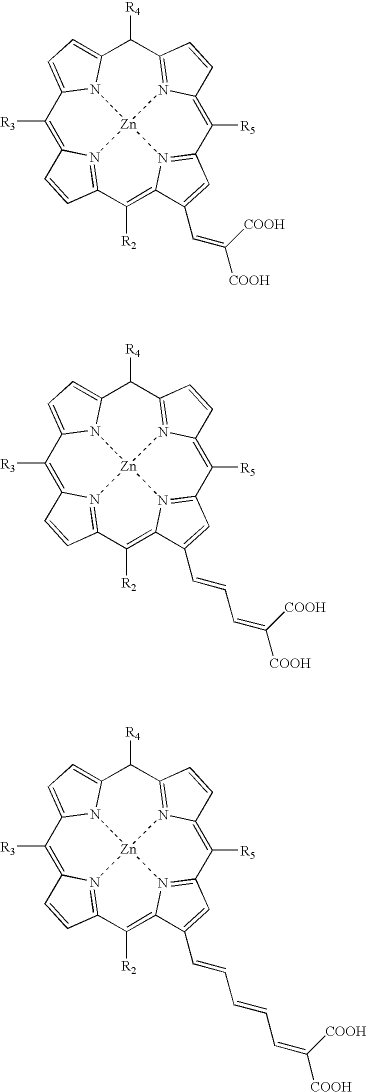

where R₂, R₃, R₄ and R₅ are tert-bityl, phenyl, methylphenyl, ethylphenyl, dimethylphenyl (xylyl), tert-butylphenyl, octylphenyl, di-tert-butylphenyl, or methoxyphenyl.

In a second aspect the invention provides a dye where the dye has the structure:

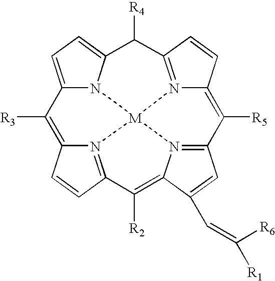

and where:
R₁ is selected from the group consisting of: carboxylates, phosphonates and sulphonates or free acids thereof;
R₂, R₃, R₄ and R₅ are independently selected from the group consisting of: H, substituted or unsubstituted alkyl, substituted or unsubstituted aryl and substituted or unsubstituted alkyl aryl;
R₆ is selected from the group consisting of: H, CN or —COOH; and
M is absent (and the porphin exists in the free base, protonated diacid, or dianion form) or is selected from the group consisting of: Cu, Ni or Zn.

Preferably the porphin of the dye exists in the metallated form. More preferably the porphin of the dye is metallated with Zn.

Preferably R₁ is a carboxylate selected from the group consisting of: cyanoacetates, malonates, or free acids thereof.

Preferably R₂, R₃, R₄ and R₅ are independently selected from the group consisting of: tert-butyl, phenyl, methylphenyl, methoxyphenyl, ethylphenyl, dimethylphenyl (xylyl), tert-butylphenyl, octylphenyl, di-tert-butylphenyl, and methoxyphenyl.

Preferably R₆ is selected from the group consisting of: H or CN.

In a first embodiment the dye is a cyanoacetic acid and selected from the group consisting of:

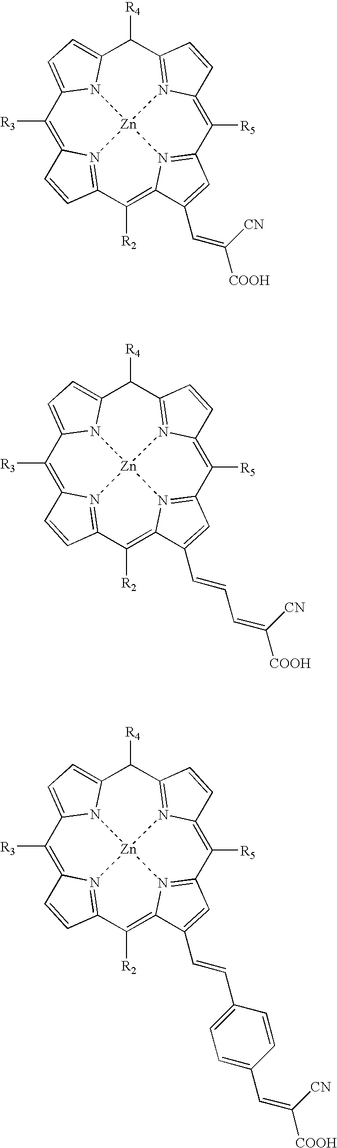

where R₂, R₃, R₄ and R₅ are tert-butyl, phenyl, methylphenyl, ethylphenyl, dimethylphenyl (xylyl), tert-butylphenyl, octylphenyl, di-tert-butylphenyl, or methoxyphenyl.

In a second embodiment the dye is a malonic acid and selected from the group consisting of:

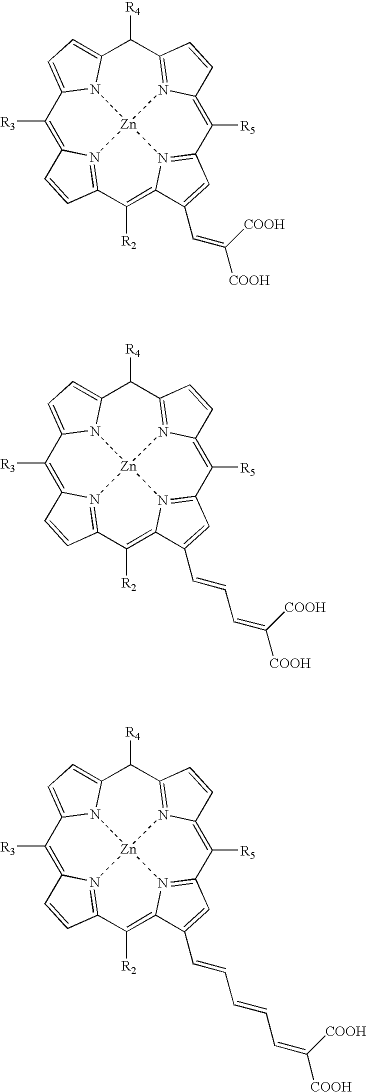

where R₂, R₃, R₄ and R₅ are tert-butyl, phenyl, methylphenyl, ethylphenyl, dimethylphenyl (xylyl), tert-butylphenyl, octylphenyl, di-tert-butylphenyl, or methoxyphenyl.

In a third aspect the invention provides a solid state photovoltaic window comprising nanocrystalline TiO₂ dye sensitised with a dye of the second aspect of the invention and an overall conversion efficiency of at least 2.5%. Most preferably the photovoltaic window has an overall conversion efficiency of at least 3.0%.

“Acid porphyrin dye” means a porphyrin dye where the substituent at the β-pyrollic carbon(s) of the porphin nucleus is an acid, e.g. carboxylic acid or benzoic acid.

“β-substituted porphyrin” means a substituted porphin including a substituent at the β-pyrollic carbon(s) of the porphin nucleus where the porphin exists in the free base, protonated diacid, dianion or metallated forms.

“Bound” means by an ester formation, coordination (syn-syn bridging), chelating, or H-bonding interaction between an acid function of the β-substituted porphyrin and the semiconductor surface.

“Carboxylic acid” means a compound (or substituent) having one or more carboxyl radicals and phosphonic acid and sulfonic acid have corresponding meanings.

“Hole conducting material” means a material that allows the regeneration of the porphyrin dye after electron injection in to the conduction band of the semiconductor due to its hole transport properties.

“Non-acceptor” means a substance used to coat the semiconductor surface to raise the conduction band potential at the electrode-electrolyte interface.

As used in the Statements of Invention and Claims the phrase “overall conversion efficiency” means the conversion efficiency measured in a solid state photovoltaic window normalised to provide a corrected “overall conversion efficiency” of 3.0% for ZnTPP-=-=(CO₂H)₂.

The following abbreviations are used:
AFM atomic force microscopy
AM1.0 air mass 1.0 (shortest path length for solar radiation through the atmosphere, 1000 Wm⁻²)
AM1.5 air mass 1.5 (1.5 times the shortest path length for solar radiation through the atmosphere, 1000 Wm⁻²)
AR analytical reagent
app apparent
aq. aqueous
Ar aryl group
avg average
Au gold
BAP 5,15-bis-aryl-porphyrin
BAcP bis-acetal-porphyrin
BCP bis-carboxy-porphyrin
BCMP bis-carboxy-bis-methoxy-porphyrin
BEP bis-ester-porphyrin
BDP bis-disulfide-porphyrin
BFP bisformyl-porphyrin
BP 3,5-di-tert-butylphenyl group
B2TP bis-2-thienyl-porphyrin
B3TP bis-3-thienyl-porphyrin
BTTP bis-terthienyl-porphyrin
Calcd calculated
CHCA α-cyano-4-hydroxycinnamic acid
conc. concentrated
COSY correlated spectroscopy
d doublet
DBU 1,8-diazabicyclo[5.4.0]undec-7-ene
DCE 1,2-dichloroethane
DCM dichloromethane or CH₂Cl₂
DMF N,N-dimethylformamide
DMSO dimethylsulfoxide
dia circular diameter
DPM dipyrrylmethane
EI electron ionisation
eq equivalent
ES electrospray
Et ethyl
Et₂O diethyl ether
EDW electron donating group
Et₃N triethylamine
EWG electron withdrawing group
FAB fast atom bombardment
FET field-effect-transistor
FF fill factor (ratio of the maximum output of the photovoltaic device, to the product of I_{SC} and V_{OC})
GaAs gallium arsenide
GP general-purpose reagent
h hours
hept heptet
hex hextet
HMTA hexamethylenetetranine
HR high resolution
HRMS high resolution mass spectrometry
HOMO highest occupied molecular orbital
IPCE incident photon-to-current conversion efficiency
I_{sc} short circuit current
ITO indium-tin-oxide (conductive glass coating)
LUMO lowest unoccupied molecular orbital
LR low resolution (MS) or long range (NMR)
LRMS low resolution mass spectrometry
min minutes
M mol L-1
M a metal ion
m multiplet, milli
MALDI matrix assisted laser desorption ionisation spectroscopy
Me methyl
MeOH methanol
mp melting point
MS mass spectrometry
NMR nuclear magnetic resonance
[O] oxidation
oct octet
P(A-D) TiO₂ coated ITO glass (batches A-D)
Pc phthalocyanine
PEC photoelectrochemical cell
pent pentet
Ph phenyl
ppm parts per million
Pps phosphonium salt
q quartet
[R] reduction
R_{f} retention factor
RT room temperature
RO reverse osmosis
ROSEY rotating frame overhauser enhancement spectroscopy
sat. saturated
SC semiconductor
SEM scanning electron microscopy
sh shoulder
SP “sticky” porphyrin
SS steady state
STM scanning tunnelling microscopy
t triplet
TAcP tetra-acetal-porphyrin
TAP 5,10,15,20-tetra-aryl-porphyrin
TBM tetrabutyltetramethyl
TBMP 2,8,12,18-tetra-n-butyl-3,7,13,17-tetramethylporphyrin
TBP 5,10,15,20-tetrakis(3′,5′-di-tert-butylphenyl)porphyrin
TBP 5,10,15,20-tetra-n-butylporphyrin
TCA trichloroacetic acid
TCP tetra-4′-carboxy-porphyrin
T3 CP tetra-3′-carboxy-porphyrin
T3,5CP tetra-3′,5′-dicarboxy-porphyrin
TEP tetra-4′-ester-porphyrin
T3EP tetra-3-ester-porphyrin
T3,5EP tetra-3′,5′-diester-porphyrin
TFA trifluoroacetic acid
TFPP tetra-(4′-formylphenyl)porphyrin
THF tetrahydrofuran
TiO₂ titanium dioxide
TLC thin layer chromatography
TMS tetramethylsilane
TOF time-of-flight
TOPP 5,10,15,20-tetra(4-octylphenyl)porphyrin
TPP 5,10,15,20-tetraphenylporphyrin
TR all-trans-retinoic acid
TTTP tetra-3″-terthienyl-porphyrin
TXP tetra-xylyl-porphyrin or 5,10,15,20-tetrakis(3′,5′-dimethylphenyl)porphyrin
UV-vis ultraviolet-visible spectroscopy
Xyl xylyl (3,5-dimethylphenyl group)
V_{oc} open circuit voltage

The following designations are used.
ZnTPP—=CNCO₂H for:

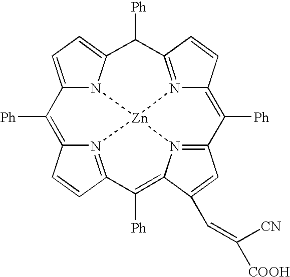

alternatively designated Zn-3.
ZnTPP-=-=CO₂H for:

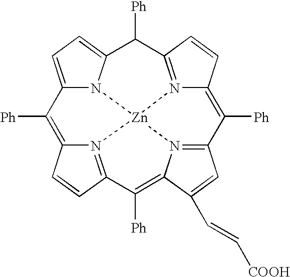

alternatively designated Zn-5.
ZnTPP-=-=CNCO₂H for:

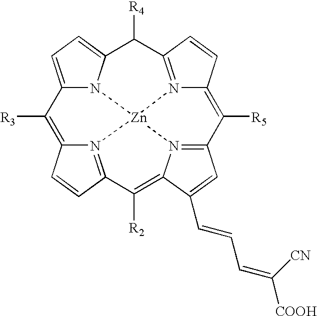

alternatively designated Zn-8.
TPP-=-Ph-3,4-(CO₂H)₂ for:

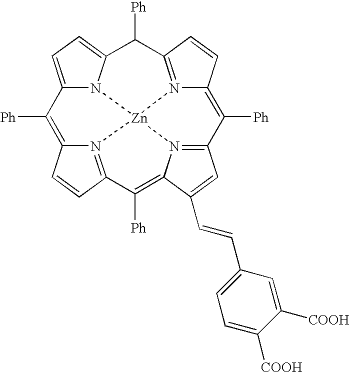

alternatively designated Zn-11.
ZnTPP-=-Ph-=CNCO₂H for:

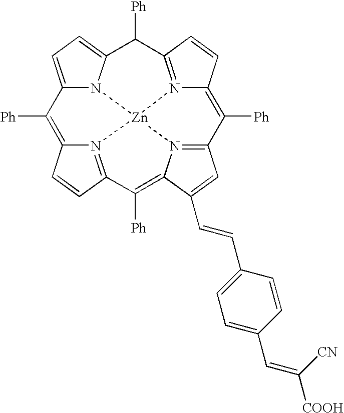

alternatively designated Zn-13.

### BRIEF DESCRIPTION OF FIGURES

The invention will now be described by way of example with reference to the accompanying drawings in which:

FIG. 1. A comparison of UV/Vis spectral properties of zinctetraphenylporphyrin (ZnTPP) with ZnTPP-=CNCO₂ (Zn-3), ZnTPP-=-CO₂H (Zn-5), ZnTPP-=-=CNCO₂H (Zn-8), TPP-=-Ph-3,4-(CO₂)₂ (Zn-11) and ZnTPP-=-Ph-=CNCO₂H (Zn-13) in THF solution.

FIG. 2. Emission spectra of ZnTPP-=CNCO₂H (Zn-3), ZnTPP-=-CO₂H (Zn-5), ZnTPP-=-=CNCO₂H (Zn-8), TPP-=-Ph-3,4-(CO₂H)₂ (Zn-11) and ZnTPP-=-Ph-=CNCO₂H (Zn-13) in THF solution.

FIG. 3. ATR-FTIR spectra of the ZnTPP-=CNCO₂H (Zn-3) obtained using solid sample (bottom) and adsorbed onto 3 μm thick TiO₂ film, which was obtained by subtracting 3 um thick TiO₂ film (top).

FIG. 4. Cyclic votammograms of 1 mM Zn-porhyrins [ZnTPP-=CHCO₂H (Zn-3), ZnTPP-=-CO₂H (Zn-5), ZnTPP-=-=CNCO₂H (Zn-8), TPP-=-Ph-3,4-(CO₂H)₂ (Zn-11) and ZnTPP-=-Ph-=CNCO₂H (Zn-13)] in DMF solution containing 0.1M TBAPF₆ with a scan rate of 100 mV/s. Working electrode is a Pt disk (2 mm in diameter); counter electrode is Pt coil; and Ferrocene is used as internal reference.

FIG. 5. Cyclic votammograms of TPP-=-Ph-3,4-(CO₂H)₂ (Zn-11) absorbed on TiO₂ film in acetonitrile containing 0.2M TBAPF₆ with a scan rate of 20 mV/s. The film thickness is 10 mm. Counter electrode is Pt coil and Ferrocene is used as internal reference.

FIG. 6. Photocurrent action spectra obtained with ZnTPP-=CHCO₂H (Zn-3) [plot 1], ZnTPP-=-=CNCO₂H (Zn-8) [plot 2], ZnTPP-=-Ph-=CNCO₂H (Zn-13) [plot 3], ZnTPP-=-CO₂H (Zn-5) [plot 4] and TPP-=-Ph-3,4-(CO₂H)₂ (Zn-11) [plot 5] anchored on nanocrystalline TiO₂ films. The incident photon to current conversion efficiency (IPCE) is plotted as a function of the wavelength of the exciting light. The spectra have been corrected according to the integrated photocurrent and the I-V measurement at 1 sun to avoid the problem caused by molecular aggregations.

FIG. 7. Photocurrent-voltage characteristics of the nanocrystalline photoelectrochemical cell sensitized with ZnTPP-=CHCO₂H. (A) 0.2 mM chenodeoxycholic acid was added in the dye solution using THF as solvent; (B) two hours' sensitization in the dye solution using ethanol as solvent. A light source simulating global AM1.5 solar radiation was employed, the incident intensity being 100 mW/cm². The insert shows the ZnTPP-=CHCO₂H coated TiO₂ film, which was used to obtain photocurrent-voltage characteristics.

FIG. 8. Photocurrent action spectra obtained for the ZnTPP-=CNCO₂H (Zn-1) and ZnTXP-=-=(CO₂H)₂ (Zn-2) sensitized heterojunction devices.

FIG. 9. Photocurrent-voltage characteristics of the nanocrystalline photoelectrochemical cell, sensitized with ZnTPP-=CNCO₂H (Zn-1) and ZnTXP-=-=(CO₂H)₂ (Zn-2) in TIE under simulated global AM 1.5 solar radiation.

### DETAILED DESCRIPTION

Many interacting factors determine the absorbance and photon-to-current conversion efficiencies of DSSC-based PECs. To date the rational design of dyes for use in the preparation of DSSCs for use in the manufacture of photoelectric devices has not been possible.

It is believed that the absorbance and photon-to-current conversion efficiencies depend on both the interaction of the sensitising dye with the surface of the semiconductor and neighbouring chromophores. These interactions are in turn influenced by the type, strength and number of anchoring groups of the sensitising dye; the type, length and number of linking groups of the sensitising dye; the spacial relationship of the chromophore of the sensitising dye to the surface of the semiconductor; chromophore assembly, e.g. discrete moieties or molecular arrays, of the sensitising dye; and inter-chromophore spacial requirements.

The ability to tune the photochemical, redox and molecular recognition properties of synthetic porphyrins offers unprecedented opportunities to develop new and more efficient sensitising dyes for the preparation of DSSCs. However, because of the many interacting factors that determine the absorbance and photon-to-current conversion efficiencies of DSSCs the ability to tune the photochemical, redox and molecular recognition properties of synthetic porphyrins in a predictive way has been limited.

Another interaction upon which photon-to-current conversion efficiency is dependent in an assembled photoelectric device such as a PEC, is that between the excited dye and the electrolyte or hole transporter. Furthermore where a liquid electrolyte or hole transporter is replaced by one in a gelled or solid state significant reductions in photon-to-current conversion efficiency are observed even for the most efficient of known ruthenium DSSCs.

Through the preparation and synthesis of a wide range of substituted porphyrin dyes the inventors have identified porphyrins substituted with a vinyl substituent at the O-pyrollic carbon(s) of the porphin nucleus (“beta substituted porphyrins”) as a class of substituted porphyrin dye that provide DSSCs with improved photon-to-current conversion efficiencies.

The low efficiency of solid state PECs incorporating DSSCs may be due to the lack of intimate contact between the hydrophilic sensitizer and the hydrophobic hole conductor. Another possibility is that there is an insufficient light absorbance resulting from the fact that the thickness of the nanocrystalline TiO₂ film on the electrode is much less than that used in the liquid-junction cell.

It has been shown for TiO₂-bound tetrakis(carboxyphenyl)-porphyrins that the efficiency of electron injection into the TiO₂ conduction band and the kinetics of electron injection and recombination are indistinguishable from those of ruthenium polypyridyl sensitizers (Y. Tachibana, S. A. Haque, I. P. Mercer, J. R. Durrant, D. R. Klug, J. Phys. Chem. B 2000, 104, 1198), although liquid-junction cells incorporating these porphyrins have to date demonstrated only moderate cell efficiencies (S. Cherian, C. C. Wamser, J. Phys. Chem. B 2000, 104, 3624).

The significant increase in cell efficiency observed for the beta substituted porphyrin sensitizers with fully conjugated carboxylate anchoring groups provides the potential to further improve conversion efficiencies of DSSCs through varying the hydrophobicity of the porphyrin sensitizer, increasing TiO₂ surface coverage through close-packing of the dyes, tuning the absorbance spectrum of the dye, and modifying the anchoring interaction of the bound dye.

From comparative, quantitative studies the inventors have now identified a subset of the beta substituted porphyrin dyes (cyanoacetic and malonic acids) that provide DSSCs demonstrating further improvements in proton-to-current conversion efficiencies, and reduced loss of these improved efficiencies when used in conjunction with gelled or solid state electrolytes or hole transporters in PECs.

### Experimental

### Porphyrin Syntheses

The syntheses of the porphyrins were achieved by combining a mixture of porphyrin Wittig chemistries using the appropriate functionalised aldehydes, integrated with classical porphyrin forming condensation reactions. The key to the efficient syntheses of the acid derivatives was to synthesise the parent compounds as ester derivatives, followed by hydrolysis as the last step to the desired acids.

Detailed description of the synthesis of specific porphyrin dyes is provided below. The following test results (without synthetic methods) are reported in Campbell et al. (Coordination Chemistry Reviews, 248, 1363-1379 (2004)) and Md. K. Nazeeruddin et al. (Langmuir, 20, 6514-6517 (2004)).

### Testing of Beta Substituted Porphyrins—Liquid Electrolyte

The test cell

Equipment and procedures were initially established to develop a semi-quantitative dye screening protocol that would provide enough information to allow promising dyes to be identified. The whole testing regime needed to be simple and allow the performance of many dye samples to be quickly evaluated.

A Grätzel cell (using a liquid electrolyte) was specifically designed and constructed for this purpose. Such a cell output is dependent on many factors other than the dye itself. In particular, it depends strongly on the thickness and quality of the TiO₂ plates and electrolyte composition used.

In order to counter these effects, a dye standard, generally the TXP derivative ZnTXP-=-Ph-4-CO₂H and later the TPP derivative ZnTPP-=-Ph-4-CO₂H, was used. The apparatus consisted simply of a 50 W halogen bulb (Philips Haltone Master Line Plus, GU 5.3, 12V/50 W Halogen bulb, 24° Beam (UV Block)) light source positioned above an X-Y-Z microscope stage, and connected to a regulated power supply.

A Darlington Phototransistor sensor was fabricated into a calibration sensor by imbedding it in an epoxy resin case. This was used prior to every test to ensure that the light intensity (c. 100 mW cm⁻²) was always consistent.

A cell holder was also fabricated to hold dye-coated TiO₂ coated conducting glass, which incorporated a light spring to hold a solid Pt counter electrode against the TiO₂ layer. The final apparatus, cell set-up and following testing procedures allowed the qualitatively screening of a large number of samples.

TiO₂ Electrode Preparation

Sections (10 mm×15 mm) of TiO₂ coated ITO glass supplied by Sustainable Technologies Australia Ltd (STA) were prepared using a cutting guide. One edge of the TiO₂ layer was then scraped back for an electrode contact point, to give a 10 mm×10 mm section.

Later plates were supplied screen-printed as 7 mm×9 mm TiO₂ sections. The TiO₂ plates were then pre-treated by washing with ethanol (30 min), hexane (30 min), Milli-Q-water (30 min) and then rinsed with ethanol again prior to drying.

Dye Adsorption

Prior to dye adsorption, the plates were fired at 490° C. for 30 min and then immersed in the dye solution while still warn. The plates were immersed in sealed containers of dye solution (2×10⁻⁴M) for overnight adsorption (12-20 h) prior to testing. UV-Vis analysis of dye adsorption indicated that complete adsorption usually occurs in 5 h.

Most dye adsorptions were carried out using about 10 ml per 1.0 cm² of TiO₂ area. The TiO₂ plates were removed from the dye solution directly before testing and the excess solvent removed by blotting on lint-free tissue paper and then drying under high vacuum for 5 min prior to cell assembly in the cell holder.

Platinum Counter Electrode Preparation

The Pt counter electrode (13 mm×12 mm) was stored in ethanol until required. The cell side was polished by rubbing on lint-free paper wetted with acetone on a flat glass surface prior to each test run.

Cell Assembly and Electrolyte Introduction

Prior and during assembly of the cell, the cell holder electrodes were short-circuited (to prevent any damaging V_{oc} conditions within the cell before testing). Following the clamping of the dyed TiO₂ electrode into the cell holder, the Pt counter electrode was placed against the TiO₂ layer while releasing the counter electrode contact support onto the Pt plate.

Electrolyte was then introduced by capillary action into the cell between the two cell electrode surfaces and any excess electrolyte removed with a paper towel. Electrolytes used in this study included: Electrolyte E (0.5 M NaI, 0.05 M I₂, in glutaronitrile); Electrolyte G (0.5 M NaI, 0.05 M I₂,4-t-butylpyridine (0.01 mol1⁻¹) in glutaronitrile); Electrolyte 1376 (0.6 M, butylmethylimidazolium iodide; BMII), 0.05 M I₂, LiI 0.1 M, 0.5 M tert-butylpyridine, 1:1 acetonitrile:valeronitrile).

Calibration of Light Source and Testing

The bulb assembly required a 15-min warm up before testing, to stabilise any thermal drift. After checking calibration of the light source the assembled cell holder was connected up to a multimeter then placed into the testing rig, in a closed circuit current (I_{sc}) reading mode.

Data Acquisition

The data collection system comprised of a Digitech Multimeter with a PC RS232C interface, collecting the data directly on a computer. I_{sc} data was recorded automatically every 30 s, and V_{oc} readings were performed for 5 s (V_{oc} reading times were kept short to eliminate any damaging open circuit conditions) as required.

V_{oc} readings were generally taken after either steady state (SS) or maximum I_{sc} value was observed. All I_{sc} values recorded in the final set-up using cell holder were corrected to mA cm⁻² by accurately measuring the TiO₂ area with Vernier calipers after testing.

Results Analysis and Errors

In general, the protocol for the initial screening of porphyrins involved the fabrication and testing of three to four separate cells for each compound. If two consistent results (<10% deviation in I_{sc}) were not obtained, extra cells were constructed and tested, calculating the average I_{sc} and V_{oc} results of these.

Any cell results that appeared as outliers were discarded; if this was a result of poor cell mechanics (i.e. short-circuiting between electrodes or damaged TiO₂ layer) generally a low V_{oc} resulted, allowing a confident exclusion of this result. Generally maximum I_{sc} values are used.

Normally ‘standard deviations’ did not exceed 8% in I_{sc}. Therefore, the errors in I_{sc} measurements are conservatively displayed as 10% errors in the following data. V_{oc} errors rarely exceeded 2%.

The Wittig-based building block approach to the development of porphyrin arrays and the concomitant introduction of surface binding functionality provided an opportunity to investigate a wide variety of electronic and structural features that could have an effect on the light harvesting ability of porphyrins in PECs.

Along with the type of solvent and concentration used for the adsorption process, a variety of general porphyrin physical and structural features were first investigated, these being the type of metalloporphyrin that would give the best cell performance, and the form of the acid moiety (i.e. salt or free acid).

Comparisons were then made with a range of mono meso-benzoic acid porphyrin dyes, followed by evaluation of the proposed array systems. Finally further optimisation of the ZnTXP-=-Ph-4-CO₂H based cell was undertaken, exploring the effect of different, o,m,p-regioisomers, steric aryl groups, electrolytes, binding groups and the importance of linker conjugation.

Initial Test Cell and Chromophore Optimisation

The solvent used to prepare the dye solutions had a significant effect on the cell performance. The solvent that gave the best cell performance for ZnTXP-=-Ph-4-CO₂H was THF and this solvent was used for all the dye comparisons. Dye concentration during the adsorption process was also important to cell performance.

For the ZnTXP-=-Ph-4-CO₂H dye, the optimum concentration appears to be somewhere between 10⁻⁴ and 10⁻⁵ M, and a standard dye concentration of 2×10⁻⁴ M was chosen. Also, in order to ascertain whether the free carboxylic acid or a salt is best for cell efficiency, the Na+ ZnTXP-=-Ph-4-CO₂H (Na) and the tetrabutylammonium salt ZnTXP-=-Ph-4-CO₂H (TBA) derivatives of the free acid form ZnTXP-=-Ph-4-CO₂H were synthesised and tested. As the free acid form ZnTXP-=-Ph-4-CO₂H gave superior cell performance, the subsequent acids were tested in their free acid form only.

TABLE 1
Salts of ZnTXP- = -Ph-4-CO₂H

| Dye | I_{SC} (mA cm⁻²) | V_{oc} (mV) |
|---|---|---|
| ZnTXP-=-Ph-4-CO₂H | 0.73 (7) | 478 |
| ZnTXP-=-Ph-4-CO₂H (Na) | 0.27 (3) | 388 |
| ZnTXP-=-Ph-4-CO₂H (TBA) | 0.066 (7) | 292 |
| Electrolyte E, PA1 Plate. | | |

It is also well known that the coordination of metal ions in the porphyrin core has an effect on the life times of the excited electronic states, which, in turn, can influence the electron transfer process between the chromophore and the conduction bands of the TiO₂. In addition, metallation affects the chemical and physical properties of porphyrins.

The Zn(II) derivative ZnTXP-=-Ph-4-CO₂H performs better than the Cu(II) CuTXP-=-Ph-4-CO₂H and the free-base species TXP-=-Ph-4-CO₂H. Both ZnTXP-=-Ph-4-CO₂H and CuTXP-=-Ph-4-CO₂H cells developed a SS behaviour in their I_{sc} values and showed good recovery in I_{sc} after a V_{oc} condition. Cu(II) porphyrins are known to have shorter lived excited states compared to the zinc porphyrins, but they are inherently more stable.

Although the CuTXP-=-Ph-4-CO₂H cell performance was half of the ZnTXP-=-Ph-4-CO₂H I_{sc} value, the results suggest that Cu(II) porphyrins may be useful where long term stability of the chromophores is required in solar cells. Based on these results and the ease of synthesis and charaterisation of Zn(II) porphyrins, all subsequent chromophores were synthesised and tested as Zn(II) metallo derivatives.

TABLE 2
Effect of metallation of TXP- = -Ph-4-CO₂H on cell performance

| Dye | I_{SC} (mA cm⁻²) | V_{oc} (mV) |
|---|---|---|
| ZnTXP-=-Ph-4-CO₂H | 1.2 (1) | 459 |
| CuTXP-=-Ph-4-CO₂H | 0.61 (6) | 460 |
| TXP-=-Ph-4-CO₂H | 0.018 (2) | 198 |
| Electrolyte E, (0.5 M NaI, 0.05M I₂, in | | |
| glutaronitrile, PA2 Plate. | | |

Comparison of β- and Meso-Substituted Monoporphyrin Carboxylic Acids

The β-substituted ZnTXP-=-Ph-4-CO₂H carboxylic acid and a range of Zn(II) meso-benzoic acid monoporphyrins were first tested in the Grätzel cell. The β-substituted ZnTXP-=-Ph-4-CO₂H monoacid, performed considerably better than all the meso-substituted porphyrin dyes. The superior performance of ZnTXP-=-Ph-4-CO₂H suggests that mode of binding is an important factor in determining cell performance. All of the porphyrins could lie flat on or edgewise to the SC surface. Edgewise binding would allow a significantly higher dye concentration on the highly uneven, porous TiO₂ surface than flat binding.

It is likely that ZnTXP-=-Ph-4-CO₂H binds predominantly edgewise given the single binding functionality. In contrast, it might be anticipated that a larger number of carboxylic functionalities might force the porphyrin to lie flat on the surface. Of the tetra-meso-acid porphyrins, Zn-T3CPP, Zn-TCPP and Zn-T3,5CPP, the meta-substituted monoporphyrin Zn-T3CPP gave significantly higher I_{sc} and V_{oc} results, with an I_{sc} value four-fold higher than the para-acid derivative Zn-TCPP, and 50% of ZnTXP-=-Ph-4-CO₂H.

Simple modelling of the tetra acids clearly demonstrates that while T3CPP could lie flat on the surface with all four acid groups available for binding, this would not be the case for TCPP. Indeed, Cherian and Wamser (J. Phys. Chem. B104 (2000)) have concluded that TCPP itself likely binds to TiO₂ in “a variety of different adsorption modes including multilayers” rather than a fully flat geometry. Therefore, the predominant factor for the improved efficiency of ZnTXP-=-Ph-4-CO₂H over Zn-TCPP could be the improved charge transfer through the fully conjugated system, along with a higher surface area coverage by this possibly edgewise-bound porphyrin.

The nearly orthogonal electronically decoupled meso-benzoic acids may limit charge injection from Zn-TCPP. In contrast, if the meta-T3CPP can adopt a fully flat binding mode, this might allow more efficient direct charge injection from the porphyrin to the SC surface, accounting for the higher performance of this tetra acid.

It was also noted that generally the low I_{sc} values of all the multiacid porphyrins were accompanied by the observation of faint dye colourations and shadowing on the TiO₂ surface after removal from the dye solution, indicating poor binding or low surface coverage. The weak binding of these acids is surprising considering the number of carboxylic acid groups (4-8) present on these molecules. However, given the potentially flatter binding modality of the multiacid porphyrins, lower surface coverage is to be expected and the rough, nanocrystalline TiO₂ may not be the optimal surface for flat binding.

In addition, Odobel et al. (J. Mater. Chem. 13, 502 (2003)) have shown that the position of substitution (meta,para) of the binding functionality has a significant influence on the sensitisation efficiency of porphyrins. It is apparent that this is indeed the case here, with the porphyrins containing meta binding functionality showing superior cell performance to those with para binding functionality. This may again be a result of the surface binding modality.

Finally, of the meso-substituted dyes, the I_{sc} for Zn-BCMPP is an order of magnitude larger than that for Zn-BCPP, and 24% of the ZnTXP-=-Ph-4-CO₂H I_{sc} value. The higher I_{sc} value for Zn-BCMPP over Zn-BCPP may possibly result from the introduction of electron donating methoxyphenyl groups to the porphyrin periphery.

TABLE 3
Mono porphyrin acids

| Dye | I_{SC} (mA cm⁻²) | V_{oc} (mV) |
|---|---|---|
| ZnTXP-=-Ph-4-CO₂H | 0.84 (8) | 453 |
| Zn-BCPP | 0.018 (2) | 118 |
| Zn-BCMPP | 0.20 (2) | 299 |
| Zn-TCPP | 0.079 (8) | 204 |
| Zn-T3CPP | 0.41 (4) | 363 |
| Zn-T3, 5CPP | 0.15 (1) | 285 |
| Electrolyte E, PD1 Plate. | | |

o,m,p-Carboxlic acids of ZnTXP-=-Ph-4-CO₂H

The effect on cell performance by the acid regioisomers of ZnTXP-=-Ph-4-CO₂H was investigated. The varying steric and electronic effects of these isomers should affect the binding ability of the dye and the relationship of the porphyrin core, to the TiO₂ surface.

It is clear that the para-Zn(II) metalloporphyrin acid ZnTXP-=-Ph-4-CO₂H performed the best, with cell performance dropping off from para, meta to ortho. If the TAP group is electron donating (reducing the acidity) the meta-acid derivative might be expected to bind stronger due to greater electronic isolation. This is however not the case, and possibly suggests surface geometry of the porphyrin is more critital.

The low I_{sc} value for the ortho-species is not unexpected, as a very light colour of porphyrin was observed on the TiO₂ surface after 10 h of adsorption, suggesting poor binding. Significant steric constraints on binding imposed by the ortho-acid group would be expected to hinder binding of this dye.

The meta-diacid ZnTXP-=-Phₘ(CO₂H)₂ gave similar I_{sc} results to the mono meta-derivative. Strong shadowing was seen on the TiO₂ layer on removal from the dye solution again implying weak binding.

TABLE 4
o,m,p-Acids of ZnTXP- = -Ph-4-CO₂H

| Dye | I_{SC} (mA cm⁻²) | V_{oc} (mV) |
|---|---|---|
| ZnTXP-=-Ph-4-CO₂H | 0.87 (9) | 422 |
| m-ZnTXP-=-Ph-CO₂H | 0.67 (7) | 436 |
| o-ZnTXP-=-Ph-CO₂H | 0.0092 99) | 209 |
| m,m-ZnTXP-=-Ph-(CO₂H)₂ | 0.73 (7) | 448 |
| Electrolyte E, PB Plate. | | |

Arylporphyrin Substituents (TPP Versus TXP Versus TBP)

It has been suggested that the lower efficiency of porphyrin sensitisers results from the increased probability of exciton annihilation from close porphyrin proximity, as a result of porphyrin aggregation. Aggregation can be significantly diminished by increasing the steric interactions between porphyries through the attachment of bulky aryl substituents. It might be anticipated therefore, that if aggregation is a problem, then replacing the meso-aryl groups of ZnTXP-=-Ph-4-CO₂H with larger groups should improve cell performance.

The performance of PECs containing TXP and TBP derivatives (ZnTXP-=-Ph-4-CO₂H and ZnTBP-=-Ph-4-CO₂H) were found to be comparable, but surprisingly the TPP ZnTPP-=-Ph-4-CO₂H derivative gave a significantly higher I_{sc} value. A second set of cells were run at a later date using a new Electrolyte 1376 on a different batch of TiO₂ plates (PE). These results also showed no significant differences in I_{sc} between the TXP and TBP derivatives and the ZnTPP-=-Ph-4-CO₂H still being superior.

It is tempting to infer from these results that closer porphyrin proximity enhances light harvesting on the SC surface. This might not be surprising given the nature of photosynthetic light harvesting complexes. However, the meso-aryl substituent significantly varies the electronic nature of the porphyrin and attached functionality, as we have observed through the change in reactivity of various porphyrin styryl benzaldehydes.

This improved performance may be entirely due to electronic tuning of the porphyrin orbitals to better interact with the TiO₂ conduction band. Nonetheless, it is not unreasonable to draw the conclusion from these observations that close proximity of porphyrins may not significantly diminish light harvesting.

Given the improved light harvesting performance of the TPP derivative ZnTPP-=-Ph-4-CO₂H, it was used as the new reference compound and the base porphyrin core in future porphyrin synthesis.

TABLE 5
TXP (ZnTXP- = -Ph-4-CO₂H) vs. TPP (ZnTPP- = - Ph-4-CO₂H) vs.
TBP (ZnTBP- = -Ph-4-CO₂H) derivatives

| Dye | I_{SC} (mA cm⁻²) | V_{oc} (mV) |
|---|---|---|
| ZnTXP-=-Ph-4-CO₂H | 0.73 (7) | 423 |
| ZnTPP-=-Ph-4-CO₂H | 1.1 (1) | 434 |
| ZnTBP-=-Ph-4-CO₂H | 0.64 (6) | 403 |
| Electrolyte G, PD2 Plate. | | |

Electrolyte Influence

The nature of the electrolyte has a significant impact on the cell performance. As indicated earlier, it was observed that the current generated from the Cu(II) TXP derivative of M-1a was 50% less than that of the Zn(II) derivative when using Electrolyte E on PA2 plates. This is also the case with Electrolyte 1376, which gives superior performance for the Zn(II) TPP derivative ZnTPP-=-Ph-4-CO₂H. Surprisingly, CuTPP-=-Ph-4-CO₂H has a very small I_{sc} output in Electrolyte 1376 yet still maintains a good V_{oc}. In Electrolyte G the overall cell performance is down, but there is no differentiation between the I_{sc} values of the two different metallostates.

TABLE 6
Electrolyte effect

| | Electro- | | |
|---|---|---|---|
| Dye | lyte | I_{SC} (mA cm⁻²) | V_{oc} (mV) |
| ZnTPP-=-Ph-4-CO₂H | G | 3.1 (3) | 442 |
| | 1376 | 5.3 (5) | 597 |
| CuTPP-=-Ph-4-CO₂H | G | 3.1 (3) | 505 |
| | 1376 | 0.054 (5) | 505 |
| Electrolyte G, (0.5 M NaI, 0.05 M | | | |
| I₂, 4-t-butylpyridine (0.01 mol⁻¹) | | | |
| in glutaronitrile), Electrolyte | | | |
| 1376 (0.6 M, butyl-methyl- | | | |
| imidazolium iodide, BMII), | | | |
| 0.05 M I₂, LiI 0.1 M, 0.5 M tert- | | | |
| butylpyridine, 1:1 acetonitrile: | | | |
| valeronitrile) (PE Plate). | | | |

Binding Groups

It was apparent throughout these studies that the degree of binding of the porphyrin dyes to the SC surface was highly variable and undoubtedly reflected the electropic influence of the porphyrin moieties on the acid pKa values.

With the best dye ZnTPP-=-Ph-4-CO₂H, the dye-coated plates discoloured when they were rinsed in the adsorption solvent. In addition, on removal of the TiO₂ plates from the dye solution, a coloured patterning, known as “shadowing” was observed as the solvent front dried and carried dye across the TiO₂ surface.

These observations suggest that ZnTPP-=-Ph-4-CO₂H is not strongly bound to the TiO₂. In contrast, the Ru dyes, such as N3 and Black Dye, are known to bind strongly to TiO₂ and are not removed by rinsing. This is presumably due to the greater acidity of the Ru dyes (pKa <3.3) compared to the TAP dye ZnTPP-=-Ph-4-CO₂H.

While it has not been possible to determine the acid pKa value for the latter, the porphyrin macrocycle is believed to be electron donating, leading to an acid pKa greater than that for the parent benzoic acid (pKa=4.2). Thus, a stronger binding group might be expected to increase the cell performance and consequently the sulphonic acid ZnTPP-=-Ph-4-SO₃H and phosphonic acid ZnTPP-=-Ph-4-PO₃H₂ (synthesised as the TBA salt) derivatives of ZnTPP-=-Ph-4-CO₂H were synthesised and tested.

These new acids were not removed by rinsing and displayed no “shadowing” yet the carboxylic acid derived ZnTPP-=-Ph-4-CO₂H is significantly superior in SC sensitisation to either the sulphonic or phosphonic acid porphyrins. This tends to suggest that electronic coupling between the dye and TiO₂ surface through the binding group has an important role to play in the efficiency of light harvesting.

TABLE 7
Carboxylic vs. sulphonic vs. phosphonic acid binding groups

| Dye | I_{SC} (mA cm⁻²) | V_{oc} (mV) |
|---|---|---|
| ZnTPP-=-Ph-4-CO₂H | 5.2 (5) | 597 |
| ZnTPP-=-Ph-4-SO₃H | 0.27 (3) | 364 |
| ZnTPP-=-Ph-4-PO₃H₂ (TBA salt) | 0.21 (2) | 410 |
| Electrolyte 1376, Plate PE. | | |

Linker Conjugation

One of the critical features of this work has been to demonstrate the value of the conjugative linkage between the porphyrin and the binding group. It was inferred from the results that it was this linkage that was responsible for the improved light harvesting performance of these β-substituted carboxylic acid functionalised porphyrins over the meso-aryl carboxylic acid functionalised porphyrins. Therefore, we not only prepared a variety of β-substituted carboxylic acid porphyrins with varying degrees of conjugation, but also porphyrins with the conjugation removed.

For the conjugated linkers, the number of double bonds, or the presence of the phenyl moiety, made no significant difference in overall cell performance. However, when the conjugation is interrupted, there is a significant fall in the cell performance. Both the reduced porphyrin of ZnTPP-=-Ph-4-CO₂H, ZnTPP—CH₂—CH₂-PhCO₂H and the phenyl acetic acid derivative ZnTPP-=-Ph-CH₂—CO₂H gave considerably lower I_{sc} values. Therefore, it appears clear that conjugation of the binding functionality to the porphyrin core significantly enhances solar cell performance.

Quantitative Grätzel Cell Results

The value of this approach to the screening of porphyrin dyes for PECs has been confirmed with independent quantitative testing of the best performing porphyrin dye ZnTPP-=-Ph-4-CO₂H. This dye gave a q of 4.24% (IPCEₘₐₓ=80%, I_{sc}=9 mA cm⁻², V_{oc}=654 mV, FF=0.72) under AM 1.5 conditions using Electrolyte 1376. This value is better than the best-reported literature value of η=3.5% for a TCPP TiO₂-dyed PEC.

Summary

We have shown that beta-substituted porphyrin styryl carboxylic acids are superior to meso-aryl porphyrin carboxylic acids as light harvesters. Although this work does not allow an easy rationalisation of this difference, it is clear that surface orientation, electronic communication and the electronic nature of the porphyrin all profoundly affect its light harvesting ability.

Zn(II) metalloporphyrin ZnTPP-=-Ph-4-CO₂H has been shown to be the most efficient porphyrin photosensitiser tested to date. This dye has an efficiency of η=4.2%. The porphyrin dyes of the invention have potential as alternatives to Ru-based dyes in the DSSC.

### Testing of Cyanoacetic Acid Beta Substituted Porphyrins

DSSCs for use in the preparation of photoelectric devices, e.g. solar cells, with improved light energy conversion efficiency have been identified. Further studies were then performed to provide DSSCs for use in the preparation of photoelectric devices, e.g. photodetectors, with absorbances towards the near infrared portion of the visible light spectrum. Of particular interest were beta substituted porphyrins for use in PECs including gelled or solid electrolytes or hole transporter.

The synthesis of β-substituted porphyrin carboxylic acids was achieved from the free-base (FB) β-formylporphyrin TPP-CHO using the procedures provided. The α-cyanoacetic acid derivative ZnTPP-=CNCO₂H (Zn-3) was synthesized by metallation using the acetate method to give the metallo derivative ZnTPP-CHO. Knoevenagel condensation with cyanoacetic acid gave the piperidine salt of ZnTPP-=CNCO₂H in good yield. Subsequent treatment of the salt with phosphoric acid (pH 2), gave the desired carboxylic acid ZnTPP-=CNCO₂H without demetallation.

Both the piperidine salt proton resonances/integrals of the salt and the acid proton of ZnTPP-=CNCO₂H were observable in the ¹H NMR. In the piperidine salt, a broadened base line was observed under the β-pyrrolic resonances, this was presumed due to the NH⁺ resonance.

The synthesis of the acrylic acid derivative ZnTPP-=-CO₂H has previously been reported via the Wittig reaction of the nickel β-formylporphyrin with ethyl (triphenylphosphoranylidene) acetate to give NiTPP-=-CO₂Et and subsequent demetallation of this with sulfuric acid yielded TPP-=-CO₂Et. Metallation using zinc acetate, followed by hydrolysis with potassium hydroxide gave ZnTPP-=-CO₂H.

Here a synthesis of ZnTPP-=-CO₂H was achieved by converting the FB β-formylporphyrin TPP-CHO directly to the FB porphyrin ethyl acrylic acid ester TPP-=-CO₂Et. FB TPP-CHO has also been previously synthesized by Effenberger and Strobel using the phosphonium salt, but not fully characterized.

We choose the Wittig reaction of aldehyde TPP-CHO directly with the ylide ethyl (triphenylphosphoranylidene)acetate in refluxing toluene to give TPP-=-CO₂Et in excellent yield. Isomerisation of the resulting cis/trans (2:3) mixture with 12 then gives TPP-=-CO₂Et in 90% overall yield. The metallation of TPP-=-CO₂Et to give ZnTPP-=-CO₂Et, followed by ester hydrolysis quantitatively gave ZnTPP-=-CO₂H as described by Selve et. al.

The ¹H NMR spectra of TPP-=-CO₂Et and ZnTPP-=-CO₂Et are consistent with those reported elsewhere, and the resulting acid ZnTPP-=-CO₂H has now been fully characterised by ¹H NMR, FAB HRMS and UV-visible spectroscopy.

Synthesis of the α-cyanopentadienoic acid ZnTPP-=-=CNCO₂H (Zn-8) was achieved via the DIBAL-H reduction of ester TPP-=-CO₂Et to alcohol TPP-=-CH₂OH using a similar procedure to that of Effenberger and Strobel. MnO₂ oxidation of TPP-=-CH₂OH to aldehyde TPP-=-CHO, followed by metallation gave ZnTPP-=-CHO. An alternative synthesis of aldehyde TPP-=-CHO has also been previously reported by Ishkov et. al via the Wittig reaction of β-formylporphyrin TPP-CHO with the protected ylide of 2-bromoethanal, followed by hydrolysis to an inseparable cis: trans mixture of aldehyde TPP-=-CHO. The ¹H NMR data of TPP-=-CH₂OH and TPP-=-CHO is consistent with that published. The Knoevenagel condensation of ZnTPP-=-CHO with cyanoacetic acid then gave the pentadienoic acid ZnTPP-=-=CNCO₂H in good yield (84%).

The conversion of β-formyl porphyrin TPP-CHO into the phosphonium salt allowed the synthesis of ZnTPP-=-Ph-3,4(CO₂H)₂ and ZnTPP-=-Ph-=CNCO₂H. First using the Wittig chemistry of the phosphonium salt with an excess of dimethyl 4-formylphthalate allowed the efficient synthesis of TPP-=-Ph-3,4(CO₂Me)₂, after 12 isomerisation. Metallation then hydrolysis gave ZnTPP-=-Ph-3,4(CO₂H)₂ in excellent yield.

Again the Wittig reaction of the phosphonium salt with terephthaldialdehyde affords the building block TPP--=-Ph-4-CHO. Quantitative metallation of TPP--=-Ph-4-CHO to give ZnTPP--=-Ph-4-CHO and the subsequent condensation with cyanoacetic acid gave the desired α-cyanoacetic acid product ZnTPP-=-Ph-=CNCO₂H. Generally all the final acid products were purified via precipitation from appropriate solvents. Chromatography of the acids was avoided at all costs, as experience showed that this generally resulted irretrievable loss of these compounds.

All compounds were characterised by ¹H NMR spectroscopy, UV-visible spectroscopy and FAB HRMS. All the ¹H NMR spectra were fully assigned and where necessary aided by 2D NMR spectra. Often a small long-range 4J coupling 0.8 Hz was observed between the closest vinylic proton at the nearest β-pyrrolic proton on the porphyrin ring. Also in the majority of cases the broadened acid proton resonance was readily visible (and integrateable) in d6-DMSO. The HRMS data was as expected for the desired compounds.

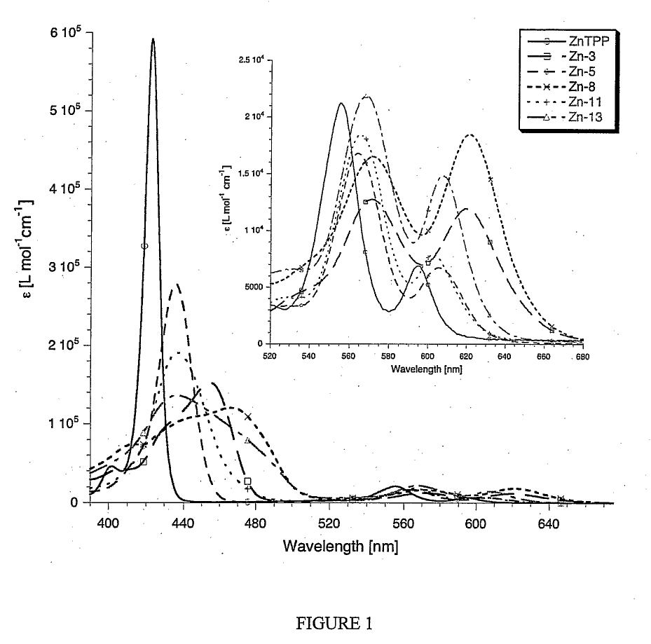

The UV/V is spectral data of metalloporphyrins ZnTPP-=CNCO₂H, ZnTPP-=-CO₂H, ZnTPP-=-=CNCO₂H, TPP-=-Ph-3,4-(CO₂H)₂ and ZnTPP-=-Ph-=CNCO₂H were measured in THF solvent. These metalloporphyrins in THF solution show a series of visible bands between 400 to 650 nm due to π-π absorptions of the conjugated macrocycle (FIG. 1). In contrast, the UV visible spectra for the acids show significant differences, with red shifts in the Soret and Q-bands relative to ZnTPP (FIG. 1). In particular, the α-cyanoacetic acids ZnTPP-=CNCO₂H, ZnTPP-=-=CNCO₂H and ZnTPP-=-Ph-=CNCO₂H display large broadening of their Soret band adsorptions, extending over a range of 100 nm from 400 through to 500 nm. The Soret and the Q-band maxima in metalloporphyrin ZnTPP-=CNCO₂H red shifted significantly compared to the metalloporphyrin ZnTPP-=-CO₂H due to the addition of cyano group. The molar extinction coefficient of the Soret band is lower in ZnTPP-=CNCO₂H compared to the other metalloporphyrins. Interestingly the Q-bands in ZnTPP-=CNCO₂H are quite intense compared to the metalloporphyrins (ZnTPP-=-CO₂H, ZnTPP-=-=CNCO₂H, TPP-=-Ph-3,4-(CO₂H)₂ and ZnTPP-=-Ph-=CNCO₂H), which we assign to the electron withdrawing nature of cyano group next to the carboxylic anchoring moiety.

The absorption spectra of the ZnTPP-=CNCO₂H, ZnTPP-=-CO₂H, ZnTPP-=-=CNCO₂H, TPP-=-Ph-3,4-(CO₂H)₂ and ZnTPP-=-Ph-=CNCO₂H adsorbed on a 9 thick TiO₂ electrodes are similar to the solution spectra with a red shift of few nm due to the interaction of the anchoring groups to the surface. The emission spectra of metalloporphyrins ZnTPP-=CNCO₂H, ZnTPP-=-CO₂H, ZnTPP-=-=CNCO₂H, TPP-=-Ph-3,4-(CO₂H)₂ and ZnTPP-=-Ph-=CNCO₂H were obtained at room temperature by exciting at 600 nm in THF solution. The emission maxima are independent of the excitation wavelength between 400 to 650 nm and the spectra show characteristic vibranic bands for ZnTPP-=-CO₂H, TPP-=-Ph-3,4-(CO₂H)₂ and ZnTPP-=-Ph-=CNCO₂H at around 628 and 671 nm, which is similar to the reported Zn porphyrins. However, the porphyrins that have α-cyano groups ZnTPP-=CNCO₂H and ZnTPP-=-=CNCO₂H display a broad single band at 668 and 678 nm, respectively.

Interestingly, the ZnTPP-=-Ph-=CNCO₂H porphyrin that also contains α-cyano group exhibits two vibranic bands at 626 and 662 nm. The difference in the emission spectra of ZnTPP-=CNCO₂H and ZnTPP-=-=CNCO₂H and ZnTPP-=-Ph-=CNCO₂H is due to the increased distance of the cyano group in the later case compared to the former two, which is consistent with electrochemical data. When exited within the emission maxima at 676 nm, the excitation spectrum exhibits an intense Soret and Q-bands, which correspond to the ground state absorption spectrum indicating the presence of a single emitting species.

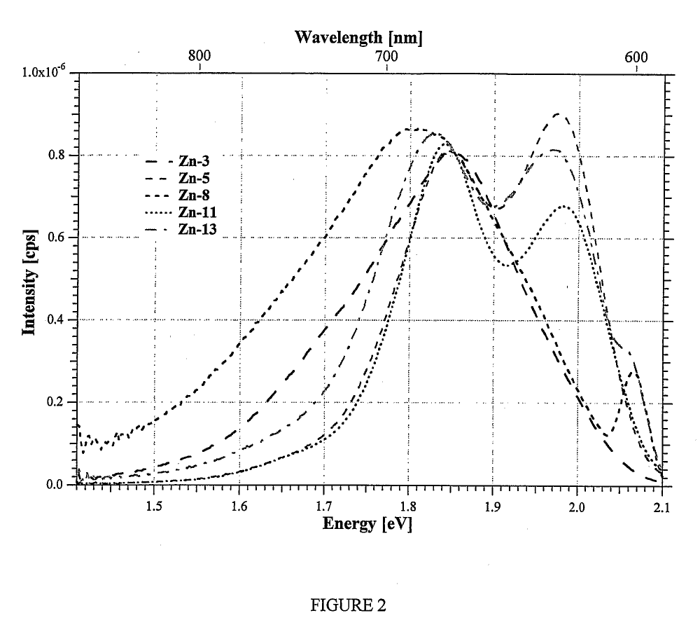

FIG. 2 shows emission spectra of metalloporphyrins ZnTPP-=CNCO₂H, ZnTPP-=-CO₂H, ZnTPP-=-=CNCO₂H, TPP-=-Ph-3,4-(CO₂H)₂ and ZnTPP-=-Ph-=CNCO₂H in THF solution at room temperature. The emission spectrum of the adsorbed metalloporphyrins ZnTPP-=CNCO₂H, ZnTPP-=-CO₂H, ZnTPP-=-=CNCO₂H, TPP-=-Ph-3,4-(CO₂H)₂ and ZnTPP-=-Ph-=CNCO₂H on a 9 μm thick TiO₂ layers are significantly quenched as a result of electron injection from the excited singlet state of the porphyrin into the conduction band of the TiO₂. Argon-degassed solution of ZnTPP-=CNCO₂H, ZnTPP-=-CO₂H, ZnTPP-=-=CNCO₂H, TPP-=-Ph-3,4-(CO₂H)₂ and ZnTPP-=-Ph-=CNCO₂H when excited at 532 nm π-π* absorption band at 298 K exhibit shorter lifetimes. In all these porphyrins the emission decayed as a single exponential with lifetimes of 2-5 ns in degassed THF solution where the origin of the emitting state is the same.

ATR-FTIR spectral data of metalloporphyrins ZnTPP-=CNCO₂H, ZnTPP-=-CO₂H, ZnTPP-=-=CNCO₂H, Zn-1l and ZnTPP-=-Ph-=CNCO₂H were measured as a solid and adsorbed on TiO₂ films. The characteristic stretching modes of —(COOH) and —(CN) groups are used to identify the attachment phenomenon of porphyrins on the surface. The AT TPP-=-Ph-3,4-(CO₂H)₂ R-FTIR spectra of the ZnTPP-=CNCO₂H, ZnTPP-=-CO₂H, ZnTPP-=-=CNCO₂H, TPP-=-Ph-3,4-(CO₂H)₂ and ZnTPP-=-Ph-=CNCO₂H porphyrins measured as solid sample show a strong and intense absorption at around 1700 cm⁻¹ due to the ν(C═O) of the carboxylic acid group. The strong bands at 1520, 1490, 1484, 1439, 1334 and 997 cm⁻¹ are due to the ring stretching modes of the porphyrin. The IR band at around 1595 cm⁻¹ is due to characteristic ν(C═C) stretching frequency. The α-cyanoacetic acids porphyrins ZnTPP-=CNCO₂H, ZnTPP-=-=CNCO₂H and ZnTPP-=-Ph-=CNCO₂H display a medium intense band at 2223 cm⁻¹ due to the ν(CN) group stretching frequency.

ATR-FTIR spectroscopy has been shown to be a powerful tool to extract structural information of the metal complexes adsorbed onto the TiO₂ surface. ATR-FTIR spectra of the adsorbed complex on TiO₂ film show the presence of strong carboxylate asymmetric cm⁻¹ ν(—COO⁻s) and symmetric 1383 cm⁻¹ ν(—COO⁻s) bands.

In the porphyrins, ZnTPP-CNCO₂H, ZnTPP-=-=CNCO₂H and ZnTPP-=-Ph-=CNCO₂H that contain α-cyano group display besides the carboxylate asymmetric and symmetric stretching frequencies a very weak ν(NC) band at 2214 cm⁻¹. The presence of carboxylate bands in the IR spectra of adsorbed complexes on TiO₂ testify that the carboxylic acid groups are dissociated and implicated in the adsorption on the TiO₂ surface.

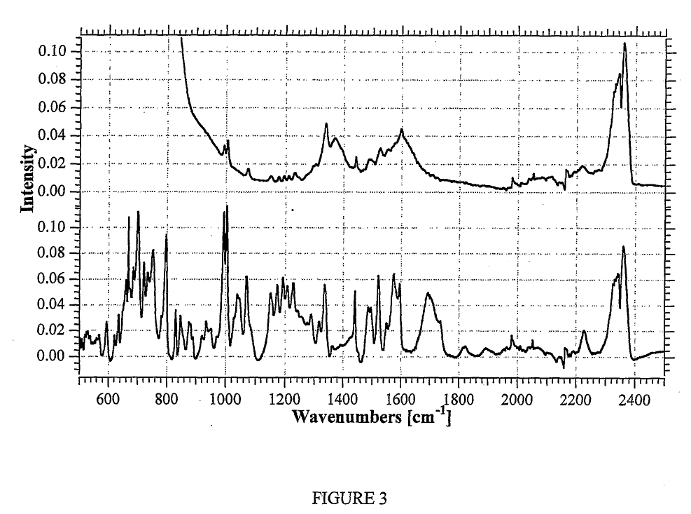

A representative ATR-FTIR spectra of ZnTPP-=CNCO₂H porphyrin measured as a solid and in the adsorbed form onto TiO₂ film were shown in FIG. 3. For sensitizers containing carboxylic acid groups several binding modes either as a unidentate and/or as a chelating and/or as a bridging bidentate are possible on the TiO₂ surface. On the basis of the IR data and the recent theoretical studies on the interaction of formic acid and sodium formate on anatase surface the unidentate and the bidentate chelation mode are ruled out, leaving the possibility of bridging coordination mode.

The decreased intensity and slight shift to lower energy of the cyano group 2214 cm⁻¹ of adsorbed porphyrins ZnTPP-=CNCO₂H, ZnTPP-=-=CNCO₂H and ZnTPP-=-Ph-=CNCO₂H clearly indicate that the cyano group is interacting with the TiO₂ surface in addition to the carboxylate binding.

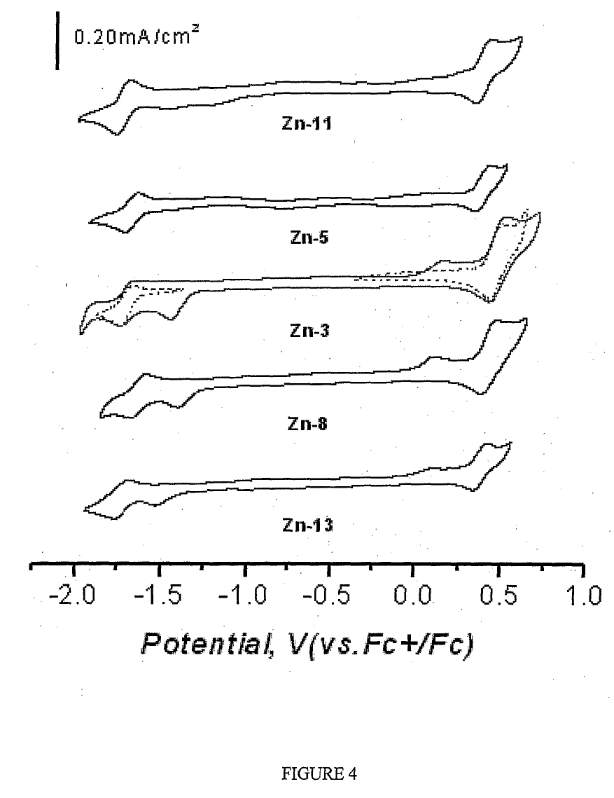

Cyclic voltammetry was used to obtain information about the energetics for formation of radical cation and anion. The CV for these Zn-porphyrins in DMF using 0.1M TBAPF₆ as supporting electrolyte is shown in FIG. 4. All porphyrins show chemically reversible waves for a one-electron reduction and an oxidation, at a scan rate of 100 mV/s. The first oxidation potential shifts slightly positively for the cynano substituted porphyrins. The oxidation potential of ZnTPP-=CNCO₂H, Eₒₓₗ=0.45V (vs.Fc+/Fc), is positively shifted by 60 mV compared with the porphyrin ZnTPP-=-CO₂H owing to the strong electron-withdrawing nature of CN-group.

In contrast, porphyrin ZnTPP-=-=CNCO₂H and ZnTPP-=-Ph-=CNCO₂H present lower oxidation potential due to the vinylene and phenyl vinylene spacer substituents, which weaken the effect from the CN-group. The TPP-=-Ph-3,4-(CO₂H)₂ shows the stepwise oxidations of two one-electron transfers (not shown). The second oxidation potential is less reversible for porphyrins ZnTPP-=CNCO₂H, ZnTPP-=-CO₂H, ZnTPP-=-=CNCO₂H and ZnTPP-=-Ph-=CNCO₂H suggesting that the radical cations are not stable.

When scanning towards negative potentials the porphyrins ZnTPP-=CNCO₂H, ZnTPP-=-CO₂H, ZnTPP-=-=CNCO₂H, TPP-=-Ph-3,4-(CO₂H)₂ and ZnTPP-=-Ph-=CNCO₂H exhibited a reversible couple at around −1.7 V, which is assigned to the reduction of porphyrin ring (FIG. 4). However, for CN-substituted porphyrins, there is an irreversible reduction peak located just before the first reversible reduction, and an induced irreversible oxidation peak during the reverse scan, which we assign to the reduction of cyano group. However, this reduction is not influencing electron injection processes because its energy is above the conduction band of TiO₂.

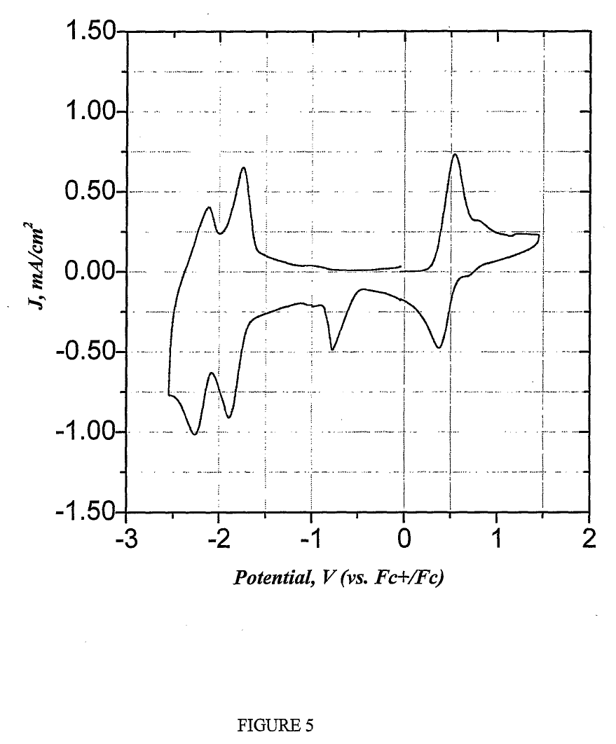

An electrochemical study of porphyrins on nanocrystalline TiO₂ film is helpful to scrutinize the electronic interactions among porphyrin molecules. Here we studied the electrochemical responses of porphyrin molecules absorbed on nanocrystalline TiO₂. A representative CV of porphyrin TPP-=-Ph-3,4-(CO₂H)₂ adsorbed on nanocrystalline TiO₂ film is shown in FIG. 5.

It is interesting to note that the redox potentials of porphyrins adsorbed on nanocrystalline TiO₂ films are similar to those in solution. However, there are two one-electron reductions as the potential is negative enough. Generally, nanocrystalline TiO₂ film are conductive only in the accumulation regime and are electronically blocking under reverse bias due to the electronic band gap. Thus, charge-transfer reactions should be restricted to adsorbed species whose redox potential lies above the conduction band edge.

For TiO₂, the flat band potential (V_{fb}) in aprotic solvents such as acetonitrile is very negative, but is tunable in the presence of protons. The oxidation potential is around 0.5V(vs.Fc+/Fc), indicating that the oxidation of porphyrins is not from the direct electron transfer between absorbed molecules and the substrate.

Our group has studied the lateral electron hopping between electroactive species on nanocrystalline oxides, where triphenylamine and fullerene prevent lateral hopping of hole and electron at given potentials, respectively. Owing to the high-delocalized structure, the electronic communication between porphyrin molecules in self assembled multilayer has been observed.

From the oxidation peaks of CV in FIG. 5, it is shown that lateral electron hopping is also favorable among porphyrin molecules absorbed on nanocrystalline TiO₂ film, indicating the effective overlapping with neighbouring of the nearest molecules. Except the reversible redox peaks, there is also a small irreversible peak around −0.8V (vs. Fc+/Fc) for porphyrin TPP-=-Ph-3,4-(CO₂H)₂.

According to our study of the lateral electron hopping among conjugated perylene-triphenylamine-thiophen molecules, where similar peak was also observed. this is attributed to the reduction of the separated porphyrins (partly caused by the insertion of the counter ion) via surface states other than the reduction of protons, since TiO₂ is partially conductive at this potential.

Light induced charge separation between Porphyrin/TiO₂ interface. Like conventional molecule/semiconductor heterojunction in dye-sensitized solar cell, excitation of the porphyrin molecules with visible light leads to an electronically excited state that undergoes electron-transfer quenching, injecting electrons into the conduction band of the semiconductor.

The oxidized porphyrin is subsequently reduced back to the ground state (S) by the electron donor (I—) presented in the electrolyte. The electrons in the conduction band collect at the back current collector and subsequently pass through the external circuit to arrive at the counter electrode.

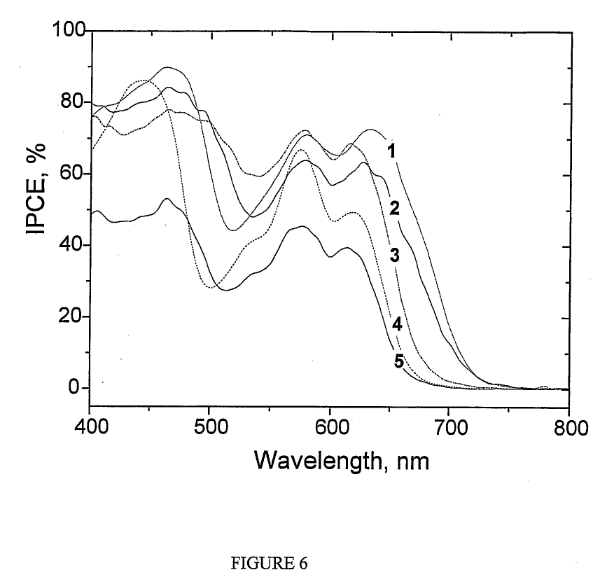

Photocurrents generated by the cell were measured as a function of wavelength in the 400-800 nm region. FIG. 6 indicates the IPCE curves versus wavelength. Porphyrins ZnTPP-=CNCO₂H, ZnTPP-=-CO₂H, ZnTPP-=-=CNCO₂H, TPP-=-Ph-3,4-(CO₂H)₂ and ZnTPP-=-Ph-=CNCO₂H have similar shape of IPCE curves. The cutoff occurs at around 700 nm. The shape of the photocurrent action spectrum is slightly broader but clearly follows the shape of the absorption spectrum of porphyrins.

The difference of IPCE values between ZnTPP-=CNCO₂H, ZnTPP-=-CO₂H, ZnTPP-=-=CNCO₂H, TPP-=-Ph-3,4-(CO₂H)₂ and ZnTPP-=-Ph-=CNCO₂H clearly indicates the effect of the anchoring group. The 3,4-carboxylic group in TPP-=-Ph-3,4-(CO₂H)₂ is spatially favorable to the adsorption of the molecules onto TiO₂ film and is beneficial to the interfacial electron injection. In addition, according to the electrochemical measurements, the stronger electron-withdrawing behavior of the 3,4-carboxylic group is also advantageous for fast dye regeneration and effective electron injection.

The higher photocurrent (J_{sc}) renders porphyrin ZnTPP-=CNCO₂H possessing much higher light-to-electricity conversion efficiency compared to the other Zn-porphyrins. The effect of CN-substituent is apparent from the photocurrent action spectra of porphyrins ZnTPP-=CNCO₂H, ZnTPP-=-CO₂H, ZnTPP-=-=CNCO₂H, TPP-=-Ph-3,4-(CO₂H)₂ and ZnTPP-=-Ph-=CNCO₂H. The CN-group in porphyrin ZnTPP-=CNCO₂H makes the spectrum to red-shifts by 50 nm and extends to 750 nm, which almost covers the visible spectrum.

The maximum IPCE value achieved at around 463 nm (Soret-band) is more than 85%, which corresponds to almost unity quantum yield (electrons per absorbed photon) if light losses are taken into account. The IPCE value of more than 70% is also obtained at the Q-band. Photocurrent of 13.5 mA/cm² has been achieved at 1 sun (AM1.5) from I-V measurement. In comparison, the cutoff wavelength of porphyrin ZnTPP-=-CO₂H, is 700 nm and the obtained photocurrent is only 9.3 mA/cm².

The CN-group is an effective auxocbrome in dye chemistry. Here, except for the enhancement of extinction coefficient, the absorption threshold of porphyrin ZnTPP-=CNCO₂H is red-shifted since the electron-withdrawing CN-substituent decreases the excited state electron density of the macrocycle, which is responsible for the more efficient light harvesting. Furthermore, the more positive oxidation potential of porphyrin ZnTPP-=CNCO₂H also helps to accelerate the dye regeneration process.

Extending the π-system of the dye is another approach to enhance the light harvesting (not only increase the extinction coefficient but also shift the absorption spectrum to longer wavelength) by tuning the band energy of the molecule. Here porphyrin ZnTPP-=-=CNCO₂H and ZnTPP-=-Ph-=CNCO₂H were synthesized to study the effect of conjugated vinylene and phenylvinylene substituents on the light-induced charge separation.

From the IPCE curves, it can be seen that the photocurrent action spectrum of porphyrin ZnTPP-=-=CNCO₂H almost extends to 750 nm, which is similar to that of porphyrin ZnTPP-=CNCO₂H. However, the IPCE value of ZnTPP-=-=CNCO₂H at the Q-band drops greatly resulting decreased integrated photocurrent, which is only 11.4 mA/cm². In comparison, the Q-band IPCE value of porphyrin ZnTPP-=-Ph-=CNCO₂H is identical to porphyrin ZnTPP-=CNCO₂H, whereas the cutoff wavelength decreases to 720 nm due to the effect of vinylene and phenylene substituent.

Although the conjugated substituents delocalize the macrocycle of the porphyrin, the distance between the macrocycle and the CN-group weakens the effect from CN-group compared to the porphyrin ZnTPP-=CNCO₂H. Therefore a blue shift for porphyrin ZnTPP-=-Ph-=CNCO₂H compared to the ZnTPP-=CNCO₂H is not surprising because the effects of vinylene and phenylene substituent is less predominant than CN-group.

Open-circuit photovoltage (V_{oc}) is an important parameter for the photovoltaic performance of molecule/semiconductor hetero-junction. Theoretically, V_{oc} is determined by the quasi-Fermi level of semiconductor and the redox potential of electrolytes. However, there are many factors, such as the surface adsorbed species (e.g. H⁺, Li⁺, etc), back reaction rate as well as recombination rates influences the value of V_{oc} in practice. The low V_{oc} of porphyrin TPP-=-Ph-3,4-(CO₂H)₂ can be attributed to the presence of two protons of the two carboxylic acid groups. As indicated in Table 2, V_{oc} of porphyrin TPP-=-Ph-3,4-(CO₂H)₂ is lower than that of porphyrin ZnTPP-=CNCO₂H from the I-V measurements. It is assumed that the possible attack of I₃⁻ on the vinylene group and the resulted higher value of Iₒ are responsible for the low V_{oc}.

For macrocyclic organic dyes, like phthalocyanine or porphyrin, molecular aggregation is one of the key issues for achieving high efficient light harvesting. Owing to the n-stacking of the aromatic rings, porphyrin molecules are subject to weak adsorption on TiO₂ film, which leads to unfavorable electron injection. The ways to prevent the aggregation include selection of proper solvent to enhance the solubility of the dye, as well as adding bulky molecule as spacer. Here two hours' sensitization of the film in the dye solution of porphyrin ZnTPP-=CNCO₂H using ethanol as solvent resulted in a conversion efficiency of 5.4%, where V_{oc}=637 mV, J_{sc}=12.1 mA/cm² and FF=0.70. Alternatively, 0.2 mM chenodeoxycholic acid was added to the porphyrin solution to alleviate the aggregation problem.

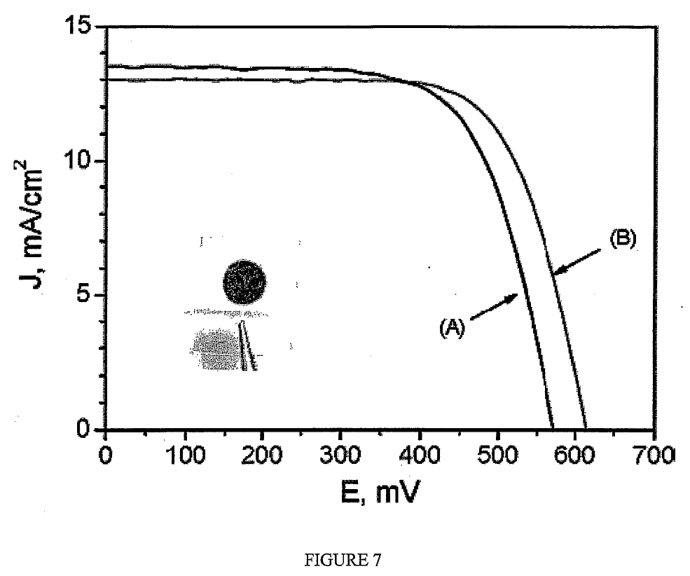

As the I-V curve indicated in FIG. 7, the modified electrode demonstrates much improve photovoltaic performance. The V_{oc} is 610 mV, J_{sc} is 13 mA/cm², and Mill factor is 0.70, yielding the overall solar (global AM 1.5 solar irradiance 100 mW/cm²) to electricity conversion efficiency of 5.6%.

To the best of our knowledge these are the highest values reported for the porphyrin/TiO₂ heterojunction light harvesting system. Since porphyrin ZnTPP-=CNCO₂H has an intense green color like a green leaf, it is very interesting as a mimic of natural chlorophylls.

The soaking test with porphyrin ZnTPP-=CNCO₂H is promising. After light soaking at 55° C. for 200 hours, the photocurrent showed no decrease. The higher temperature stability (e.g. 80° C.) needs molecules with stronger anchoring group. The CN-substituted porphyrins with phosphonate anchoring group that have higher pKa values should be promising.

Summary

A series of novel porphyrins which show intense green color when anchored onto TiO₂ surfaces have been provided. The ground and excited state redox potentials show that the porphyrins possess suitable potentials to inject electrons into the TiO₂ substrate yielding 85% IPCE. Among the reported porphyrins, the green colored ZnTPP-=CNCO₂H shows the highest power conversion efficiency obtained so far on nanocrystalline films.

Additional comparative data is provided on the following pages.

TABLE 8
Efficiencies of cyanoacetic acid and malonic acid beta
substituted porphyrins in a solid state heterojunction.

| Compound | η (%, 1 sun)* |
|---|---|
| ZnTPP-=CNCO₂H | 2.8 |
| ZnTPP-=-=(CO₂H)₂ | 2.5 |
| ZnTTP-=-=(CO₂H)₂ | 3.6 |
| ZnTXP-=-=(CO₂H)₂ | 3.0 |
| ZnT4BP-=-=(CO₂H)₂ | 3.0 |
| ZnTBP-=-=(CO₂H)₂ | 2.8 |

*All efficiencies were obtained from solid state cells prepared at EPFL, Lausanne, Switzerland.

TABLE 9
Efficiencies of cyanoacetic acid and malonic acid beta substituted
porphyrins in a liquid junction titanium dioxide photovoltaic cell.

| | Semiquantitative | Quantitative |
|---|---|---|
| Compound | η (%, 1 sun)^{a} | η (%, 1 sun)^{b} |
| Ru535 bis-TBA | 3.6 | 10.4 |
| ZnTPP-=CNCO₂H | 2.8 | 5.2 |
| ZnTPP-=-=CNCO₂H | — | 4.0 |
| ZnTPP-=-Ph-=CNCOOH | — | 3.7 |
| ZnTPP-=(CO₂H)₂ | 2.6 | 5.6 |
| ZnTXP-=(CO₂H)₂ | — | 3.0 |
| ZnTPP-=-=(CO₂H)₂ | 1.9 | 5.0 |
| ZnTXP-=-=(CO₂H)₂ | 2.5 | 6.1 |
| ZnTTP-=-=(CO₂H)₂ | 2.7 | 5.8 |
| ZnTEP-=-=(CO₂H)₂ | 3.0 | — |
| ZnTnBP-=-=(CO₂H)₂ | 3.3 | — |
| ZnT4BP-=-=(CO₂H)₂ | 2.0 | 5.9 |
| ZnTBP-=-=(CO₂H)₂ | 2.1 | 5.6 |
| ZnTnOctylPP-=-=(CO₂H)₂ | 2.9 | — |

^{a}Semiquantitative efficiencies were obtained from unsealed liquid junction titanium dioxide photovoltaic cell prepared at Massey University, Palmerston North New Zealand according to the experimental details given on p. 23 using the following electrolytes: Electrolyte E-Zn2 (0.1 M LiI, 0.05 M I₂, 0.5 M 4-tert-butylpyridine, 0.6 M BMII in 1:1 valeronitrile:glutaronitrile); Electrolyte E-Zn3 (0.1 M LiI, 0.05 M I₂, 0.5 M 4-tert-butylpyridine, 0.6 M BMII, 0.5 M BHT in 1:1 valeronitrile:glutaronitrile).
^{b}Quantitative efficiencies were obtained from sealed liquid junction titanium dioxide photovoltaic cell prepared at EPFL, Lausanne, Switzerland according to the experimental details given in Q. Wang, W. M. Campbell, E. E. Bonfantani, K. W. Jolley, D. L. Officer, P. J. Walsh, K. Gordon, R. Humphry-Baker, M. K. Nazeeruddin, M. Graetzel, Journal of Physical Chemistry B 2005, 109, 15397.

### Testing of Cyanoacetic and Malonic Acid Beta Substituted Porphyrins in a Solid State Heterojunction

Wittig reaction of tetraxylylporphyrin aldehyde TXP-CHO with the phosphorane, ethyl (triphenylphosphoranylidene)-acetate, resulted in a cis/trans (42% cis) mixture of the vinyl ester. Isomerization of this mixture to the all-trans ester TXP-=-CO₂Et was efficiently achieved with iodine. Oxidation of the alcohol TXP-=-COH₂OH, produced from the DIBAL-H reduction of this ester TXP-=-CO₂Et, with MnO₂ gave an excellent yield of the allylaldehyde TXP-=-CHO. A quantitative yield of the extended malonic acid derivative ZnTXP-=-=(CO₂H)₂ was obtained by malonic-acid condensation with aldehyde TXP-=-CHO, followed by metallation of the resulting product with zinc(II) acetate. The analytical and spectroscopic data for ZnTXP-=-=(CO₂H)₂ are fully consistent with the structures. The introduction of the malonic-acid group into ZnTXP-=-=(CO₂H)₂ provides a group that is a better anchoring group than the cyanoacrylic acid group.

The metalloporphyrins ZnTPP-=CNCO₂H and ZnTXP-=-=(CO₂H)₂ both show a series of absorption bands (between 400 and 650 nm) due to p-p* transitions of the conjugated macrocycle. Both compounds show red-shifts in the Soret and Q bands with respect to zinc tetraphenylporphyrin (ZnTPP) and increased molar extinction coefficients for the Q bands due to the extended conjugation and the electron-withdrawing nature of the anchoring groups. The red-shift of the Q bands in ZnTPP-=CNCO₂H compared to ZnTXP-=-=(CO₂H)₂ is due to the stronger electron-withdrawing nature of cyanide group.

The visible absorption spectra of both ZnTPP-=CNCO₂H and ZnTXP-=-=(CO₂H)₂, adsorbed on a TiO₂ film, show features similar to those seen in the corresponding solution spectra, but exhibit a small red-shift due to the interaction of the anchoring groups with the surface. The emission data of metalloporphyrins ZnTPP-=CNCO₂H and ZnTXP-=-=(CO₂H)₂ were obtained at room temperature by excitation at 570 nm in THF solution; the spectra show characteristic maxima at 670 nm. The emission time constants are several orders of magnitude greater than the electron-injection rate into the conduction band of TiO₂.

The HOMO and LUMO of ZnTPP-=CNCO₂H are at −5.62 and −3.46 eV, respectively; for ZnTXP-=-=(CO₂M)₂ they are at −5.52 and −3.56 eV, respectively. The HOMOs of both ZnTPP-=CNCO₂H and ZnTXP-=-=(CO₂H)₂ are slightly more negative than that of the standard dye cis-dithiocyanatobis(4,4′-dicarboxylic acid-2,2′-bipyridine) ruthenium(II), which makes the regeneration of the porphyrin dyes more favorable.

In addition, cyclic voltammetric measurements on porphyrin monolayers adsorbed on TiO₂ indicate that—probably as a result of the highly delocalized structure of the porphyrins and an effective overlap of neighboring molecules—lateral charge hopping takes place within the monolayer itself. This process alleviates the problem of the lack of intimate contact between the adsorbed dye and the hole conductor, since the dye can be regenerated through lateral hole hopping between dye molecules.

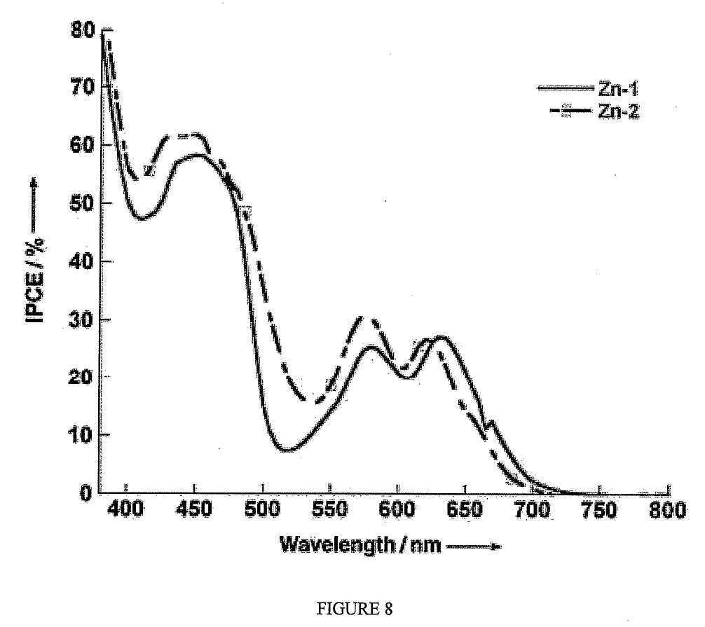

Mesoporous solid-state heterojunction cells, incorporating ZnTPP-=CNCO₂H and ZnTXP-=-=(CO₂H)₂, were prepared as previously described (U. Bach, D. Lupo, P. Compte, J. E. Moser, F. Weissgrtel, J. Salbeck, H. Spreitzer, M. Grätzel, Nature 1998, 395, 583). The photocurrent action spectra obtained from the ZnTPP-=CNCO₂H and ZnTXP-=-=(CO₂H)₂ sensitized heterojunction devices is given in FIG. 8.

The shapes of the action spectra are similar to those of the corresponding absorption spectra. For ZnTPP-=CNCO₂H, the incident monochromatic photon-to-current conversion-efficiency (IPCE) values peak at about 65% in the Soretband region, but in the Q-band region, the highest value is only 25%. In the corresponding dye-sensitized liquid-junction cell, however, the IPCE peaks are at 90% in the Soret-band region and 70% in the Q-band region. It should also be noted that the IPCE values in ZnTXP-=-=(CO₂H)₂ porphyrin-sensitized heterojunctions are substantially better than that previously obtained for the analogous ruthenium-dye-sensitized heterojunction.

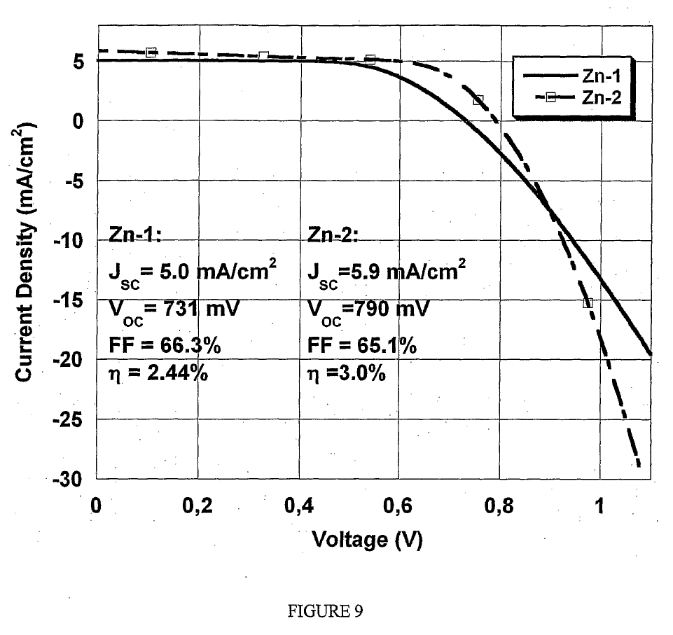

FIG. 9 show the current and voltage characteristics of the ZnTPP-=CNCO₂H and ZnTXP-=-=(CO₂H)₂ sensitized heterojunction cells under simulated global AM 1.5 light intensity (1000 W m⁻²). For the ZnTXP-=-=(CO₂H)₂ sensitized cell, the short-circuit photocurrent density (J_{sc}) of 5.9 mA cm⁻², open-circuit photovoltage (V_{oc}) of 790 mV, and fill factor (ff) of 0.65 yield an overall conversion efficiency η,derived from the equation: η=J_{sc}×V_{oc}×ff, of 3%. The corresponding values for the ZnTPP-=CNCO₂H sensitized cell are 5.1 mA cm⁻², 730 mV, 0.66, and 2.5%.

The porphyrin-sensitized cells demonstrate greatly improved efficiencies, and the porphyrin molecules themselves are readily functionalized. Further improvements in the conversion efficiencies of solid-state cells are to be anticipated by selection of the one or more of the cyanoacetic or malonic acid beta substituted porpyrins, the synthesis of which is provided below.

### Syntheses

General Procedure

¹H nuclear magnetic resonance (NMR) spectra were obtained at 270.19 MHz using a JEOL JMN-GX270 FT-NMR Spectrometer with Tecmag Libra upgrade, and at 400.13/500.13 MHz using Brucker 400/500 Avance machines running X-WIN-NMR software. The chemical shifts are relative either to tetramethylsilane (TMS) or to the residual proton signal in deuterated solvents (CDCl₃ δ 7.27, d₆-DMSO δ 3.41). ¹³C NMR shifts are relative to CDCl₃ (δ 77.0). Chemical shifts are reported as position (6), multiplicity (s=singlet, d=doublet, t=triplet, q=quartet, dd=doublet of doublets, m=multiplet), relative integral, coupling constant (J in Hz), and assignment. Full structural assignments were assisted by the acquisition of appropriate data from 2D experiments (COSY, HMQC, HMBC). UV/Vis/NIR spectra were collected on a Shimadzu UV-3101PC UV/Vis/NIR scanning spectrophotometer controlled by a Shimadzu software. AR-, HPLC-, or spectroscopy-grade solvents were used in all cases. High-resolution mass spectrometry (HRMS) (fast-atom bombardment, FAB, and electron ionisation, EI) was carried out using a Varian VG70-50S double-focusing magnetic-sector mass spectrometer. Samples analysed by FAB-HRMS were supported on an m-nitrobenzyl alcohol matrix (unless otherwise stated). The data were put through a MASSPEC II data system to give ±5 ppm error formulations on molecular ions. Major fragmentations are given as percentages relative to the base-peak intensity. Column chromatography was performed using Merck Kieselgel 60 (230-400 mesh) and thin-layer chromatography TLC was carried out using precoated silica-gel plates (Merck Kieselgel ⁶⁰F₂₅₄). The term “chromatographed” hereby implies that it (the mixture or crude product) has been subjected to either gravity or flash chromatography. Where a dual solvent system is used, gradient elution is implied, collecting the major band unless otherwise stated. All fractions or solutions containing a single spot by TLC with the same R_{f} were combined, filtered and solvent removed in vacuo (rotary evaporation followed by high vacuum), unless otherwise stated. All solid precipitates were separated by filtration or centrifugation, rinsing with the precipitating solvent, then dried under high vacuum overnight unless otherwise stated. All porphyrin reactions were in general carried out shielded from ambient light, under a nitrogen or argon atmosphere and using dry degassed solvents. The reagents and solvents used herein came from many different sources and were generally AR-grade reagents. Chromatography solvents were laboratory grade and were distilled before use. For most applications, water was treated with a reverse-osmosis filtration system. Higher purity water was obtained by distilling Milli-Q H₂O off activated charcoal. Dry degassed CH₂Cl₂ and DMF were prepared by distillation of the AR-grade solvent over CaH₂ under an N₂ atmosphere. Dry toluene, ether, benzene, and THP were prepared by passing the argon-degassed solvent through activated alumina columns. N₂ (oxygen-free) was passed through a KOH drying column to remove moisture.

### Porphyrin Cyanoacetic Acids

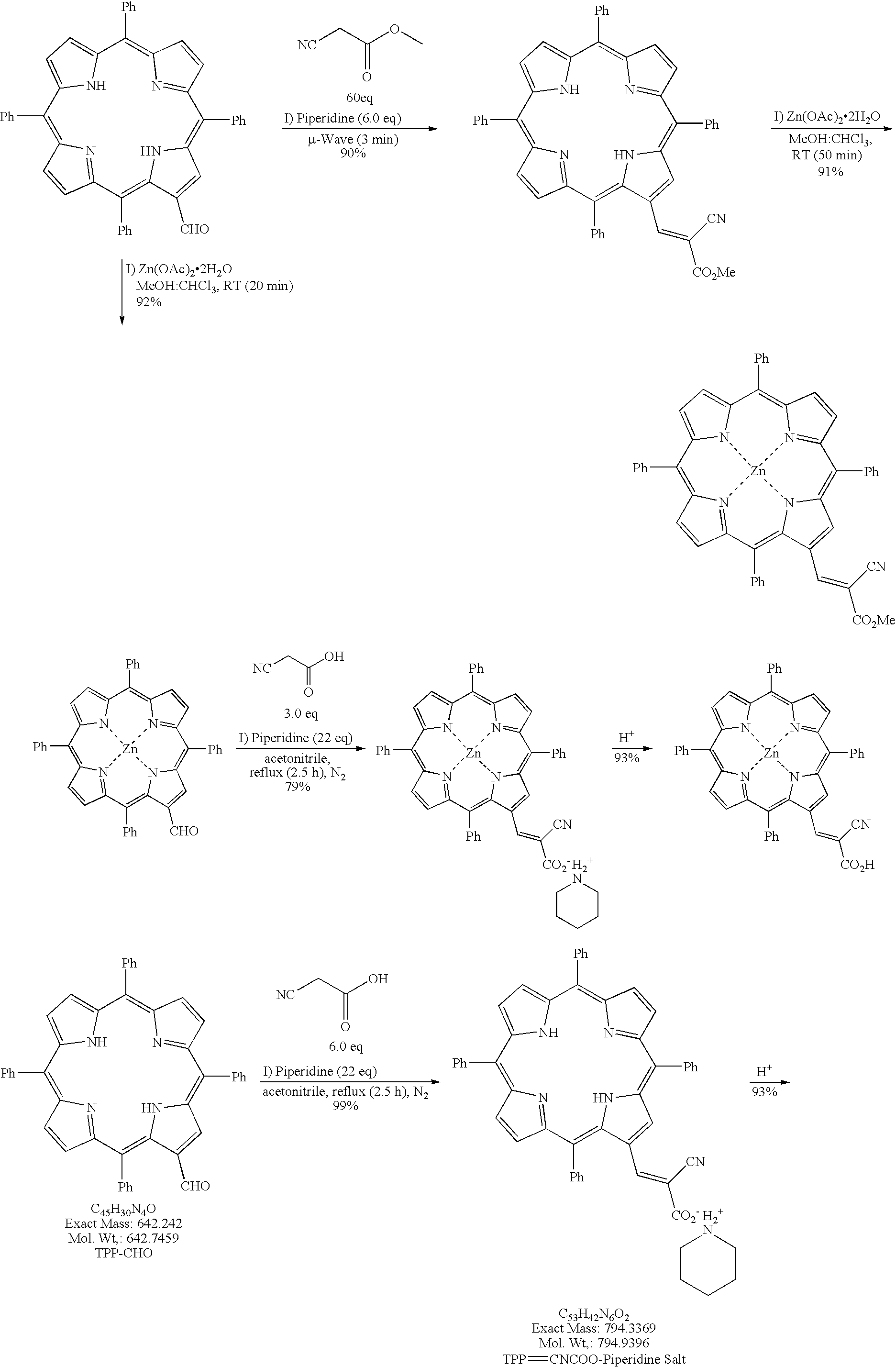

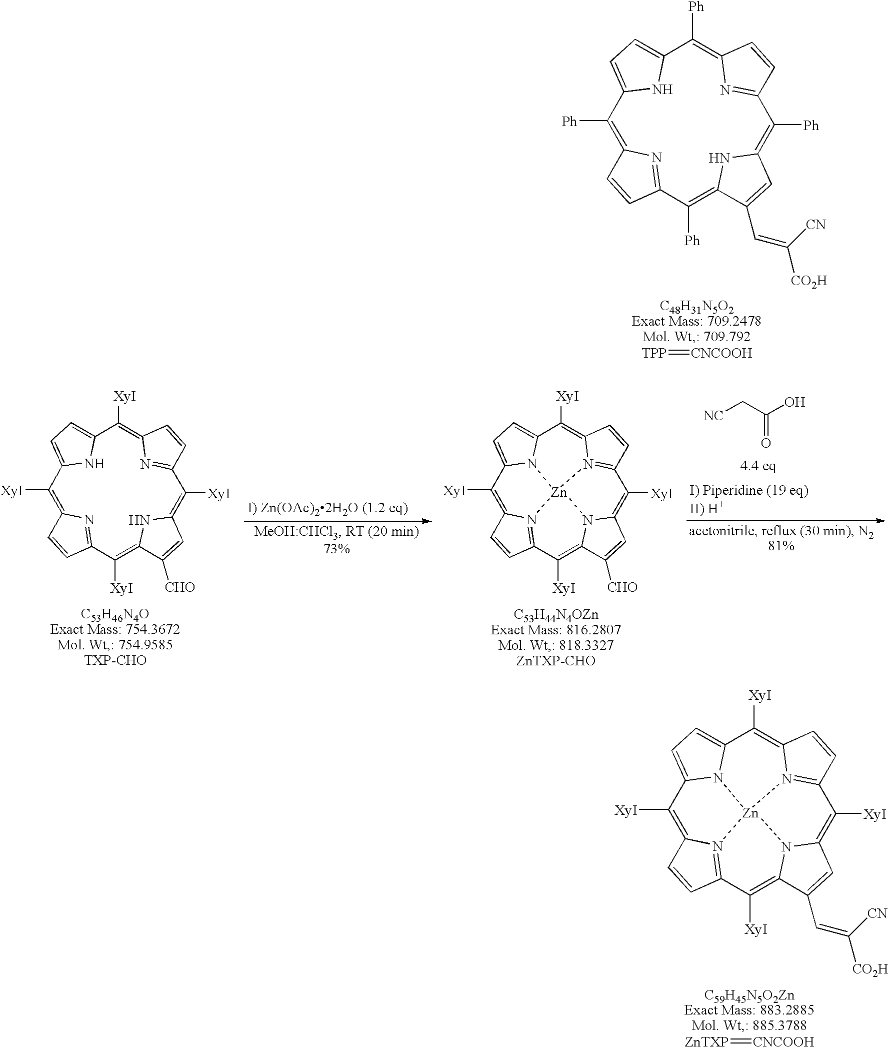

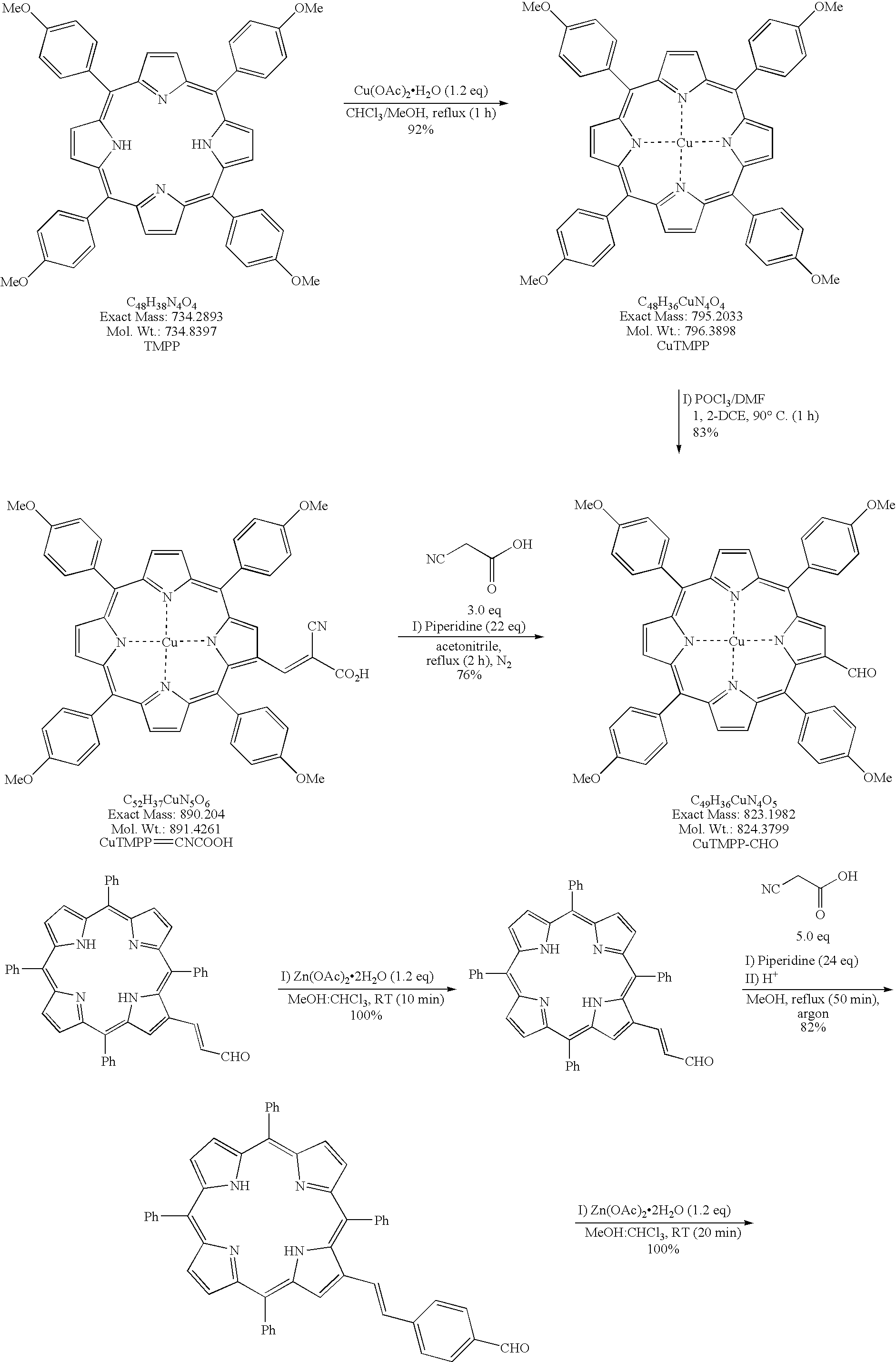

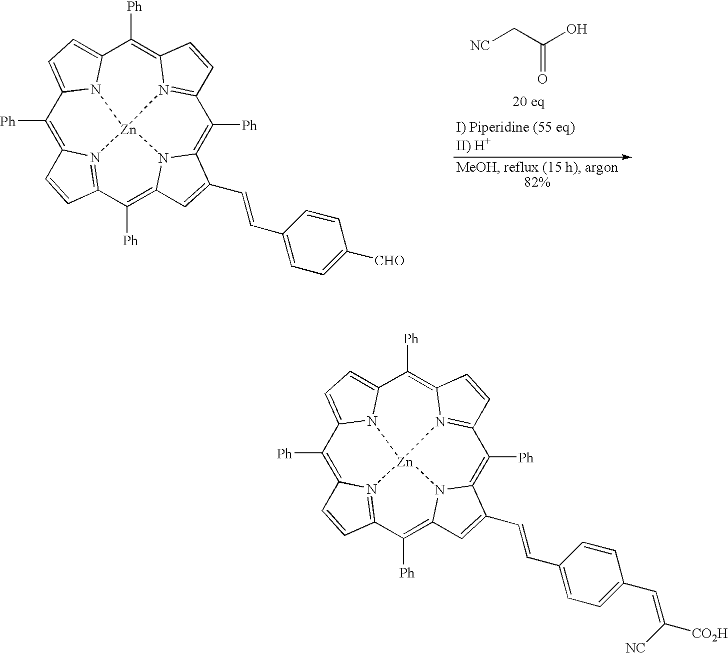

### TPP-=CNCOOMe.

### 2-Cyano-3-(5′, 10′, 15′,20′-tetraphenylporphyrin-2′-yl)-acrylic acid methyl ester

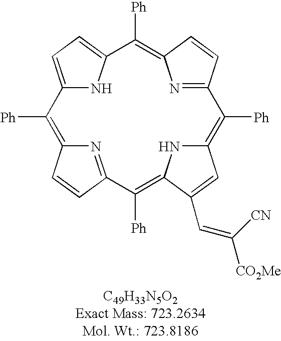

A mixture of TPP-CHO (300 mg, 466 μmol), Methyl cyanoacetate (2.5 mL, 28 mmol, 60 eq) and piperidine (300 μL, 3.0 mmol) were irradiated with μ-wave for 3 min at 50% power (Y 600 W). On cooling to RT CH₂Cl₂ (50 mL) was added and the solution filtered through paper (#1). The product was precipitated from solution with methanol to give TPP-=CNCOOMe (305.4 mg, 90%) as a purple powder.

¹H NMR (400 MHz, CDCl₃, TMS): δ-2.68 (s, 2H, NH), 3.862 (s, 3H, CO₂CH₃), 7.72-7.84 (m, 12H, H_{m.p.-Ph}), 8.066 (d, 1H, ⁴J=1.0 Hz, H₃), 8.12-8.15 (m, 2H, H_{o-Ph}), 8.18-8.22 (m, 4H, H_{o-Ph}), 8.25-8.29 (m, 2H, H_{o-Ph}), 8.743 and 8.758 (ABq, 2H, ³J=4.7, 4.7 Hz, H_{β-pyrrolic}), 8.848 and 8.864 (ABq, 2H, ³J=5.0, 5.3 Hz, H_{β-pyrolic}), 8.893 and 8.90 (ABq, 2H, 3J=4.5, 4.9 Hz, H_{β-pyrrolic}), 9.571 (d, 1H, ⁴J=1.0 Hz, H_{3′(β-pyrrolic)}). Assignments aided by COSY spectra.

UV-vis (CH₂Cl₂): λₘₐₓ [nm] (ε×10⁻³) 445 (133), 530 (14.4), 575 (5.77), 609 (5.60), 671 (7.38).

FAB-LRMS: m/z (%, assignment) cluster at 723-727, 724 (100, MH⁺). HRMS: Calcd for MH⁺ (C₄₉H₃₄N₅O₂): 724.2713, found: 724.2707.

### ZnTPP-=CNCOOMe

### 2-Cyano-3-(2′-(5′, 10′,15′,20′-tetraphenylporphyrinato zinc(II))yl)-acrylic acid methyl ester

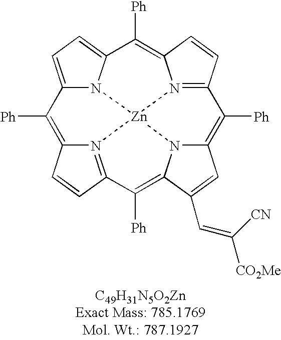

A solution of Zn(OAc)₂.2H₂O (90.9 mg, 414 μmol, 1.2 eq) in MeOH (2.5 mL) was added to a solution of TPP-=CNCOOMe (250 mg, 345 μmol) as in CHCl₃ (17 mL) with stirring at RT. After 50 min, TLC analysis indicated that the reaction was complete with the appearance of a new more polar green band (R_{f}=0.125, silica, CH₂Cl₂). The solvent was removed in vacuo and the residue was column chromatographed (silica, 37 mm_{dia}×100 mm, CH₂Cl₂). Recrystallisation from CH₂Cl₂/hexane gave ZnTPP-=CNCOOMe (247.4 mg, 91%) as a purple powder.

¹H NMR (400 MHz, CDCl₃, TMS): δ 3.693 (s, 3H, CO₂CH₃), 7.62-7.80 (m, 12H, H_{m.p.-Ph}), 7.883 (d, 1H, ⁴J=1.0 Hz, H₃), 8.02-8.05- (m, 2H, H_{o-Ph}), 8.16-8.24 (m, 6H, H_{o-Ph}), 8.825 and 8.880 (ABq, 2H, ³J=4.7, 4.7 Hz, H_{β-pyrrolic}), 8.885 (s, 2H, H_{β-pyrrolic}), 8.881 and 8.900 (ABq, 2H, ³J=4.5, 4.7 Hz, H_{β-pyrrolic}) 9.624 (d, 1H, ⁴J=1.0 Hz, H_{3′(β-pyrrolic)}). Assignments aided by COSY spectra.

UV-vis (CH₂Cl₂): λₘₐₓ [nm] (ε×10⁻³) 309 (19.5), 398 (43.5), 451 (163), 565 (13.3), 612 (14.4).

FAB-LRMS: m/z (%, assignment) cluster at 785-791, 785 (100, M⁺). HRMS: Calcd for M⁺ (C₄₉H₃₁N₅O₂Zn): 785.1769, found: 785.1755.

### ZnTPP-CHO

### 2-Formyl-5,10,15,20-tetraphenylporphyrinato zinc(II)

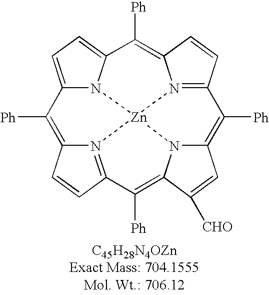

A solution of Zn(OAc)₂.2H₂O (123 mg, 560 μmol, 1.2 eq) in MeOH (3.0 mL) was added to a solution of TPP-CHO (300 mg, 467 μmol) in CHCl₃ (20 mL) with stirring at RT. After 20 min, TLC analysis indicated that the reaction was complete with the appearance of a new more polar green band (R_{f}=0.10, silica, 2:1 (CH₂Cl₂:hexane). Precipitation using MeOH gave the crude product. Recrystallisation from CH₂Cl₂/MeOH gave ZnTPP-CHO (326.6 mg, 97%) as a purple microcrystalline solid.

¹H NMR (400 MHz, CDCl₃, TMS): δ 7.71-7.83 (m, 12H, H_{m.p.-Ph}), 8.15-8.23 (m, 8H, H_{o-Ph}), 8.88-8.94 (m, 6H, H_{β-pyrrolic}) 9.224 (s, 1H, H_{3″(β-pyrrolic)}), 9.515 (s, CHO).

UV-vis (CH₂Cl₂): λₘₐₓ [nm] (ε×10⁻³) 432 (342), 526 (4.08), 560 (15.6), 602 (11.3), 681 (8.39).

FAB-LRMS: m/z (%, assignment) cluster at 704-710, 704 (100, M⁺). HRMS: Calcd for M⁺ (C₄₅H₂₈N₄OZn): 704.1555, found: 704.1542.

### ZnTPP-=CNCOOH (Piperidine Salt)

### 2-Cyano-3-(2′-(5′,10′,15′,20′-tetraphenylporphyrinato zinc(II))yl)-acrylic acid piperidine salt

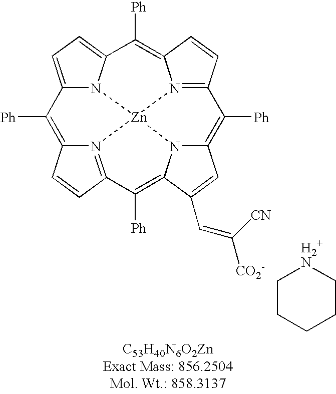

A solution of ZnTPP-CHO (250 mg, 354 μmol), cyanoacetic acid (90 mg, 1.06 mmol, 3.0 eq) and piperidine (654 μL, 7.68 mmol, 22 eq) in acetonitrile (50 mL) was heated at reflux temperature for 1 h. On cooling to RT the resulting green precipitate was collected by filtration (#4) and rinsed with acetonitrile (2.0 mL). Recrystallisation from CHCl₃(5% MeOH)/acetonitrile gave the ZnTPP-=CNCOOH (Piperidine Salt) (240.2 mg, 79%) as a purple solid.

¹H NMR (400 MHz, d₆-DMSO, TMS): δ 1.510 (br s, 6H, CH_{2, Piperidine}), 2.716 (br s, 4H CH_{2, Piperidine}), 7.67-7.82 (m, 13H, 12H_{m.p.-Ph}+1H₃), 8.01-8.04 (m, 2H, H_{o-Ph}), 8.16-8.21 (m, 6H, H_{o-Ph}), 8.64-8.79 (m, 7H, [8.635 and 8.717 (ABq, 2H, ³J=4.7, 4.7 Hz, H_{β-pyrrolic}), 8.749 (s, 2H, H_{β-pyrrolic}) 8.769 and 8.793 (ABq, 2H, ³J=4.7, 4.7 Hz, H_{β-pyrrolic})+1H⁺_{Piperidine}]), 9.298 (d, 1H, ⁴J=1.1 Hz, H_{3′(β-pyrrolic)}). Assignments aided by COSY spectra.

UV-vis (THF): λₘₐₓ [nm] (ε×10⁻³) sh 408 (41.7), 454 (150), 571 (12.6), 620 (11.5).

FAB-LRMS: m/z (%, assignment) 86 (100, piperidine), cluster at 771-778, 771 (25, M⁺).

### ZnTPP-=CNCOOH

### 2-Cyano-3-(2′-(5′,10′,15′,20′-tetraphenylporphyrinato zinc(II))yl)-acrylic acid

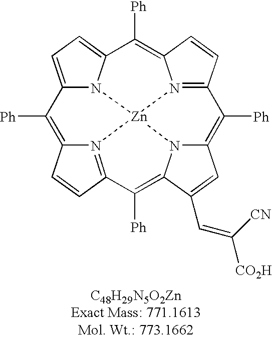

ZnTPP-=CNCOOH (Piperidine Salt) (140 mg, 163 μmol) was dissolved in DMSO-d₆ (1.5 mL). H₂O (150 mL) and CHCl₃ (50 mL) were added in separating funnel. Next 2M H₃PO₄ (2.0 mL) was added and mixture shaken vigorously (pH≈2.0). The organic layer was washed with H₂O (150 mL) containing 2M H₃PO₄ (1.0 mL) and then carefully separated. The CHCl₃ was removed by azetrope distillation using acetonitrile in in vacuo. Precipitation from the resulting acetonitrile solution (≈50 mL) using H₂O gave ZnTPP-=CNCOOH (117.7 mg, 93%) as a green powder.

### Alternative Prepation:

A solution of ZnTPP-CHO (327 mg, 463 μmol), cyanoacetic acid (118 mg, 1.39 mmol, 3.0 eq) and piperidine (855 μL, 10.2 mmol, 22 eq) in acetonitrile (66 mL) was heated at reflux temperature for 1 h. On cooling to RT the resulting green precipitate was collected by filtration (#4) and rinsed with acetonitrile (2.0 mL). The solid was dissolved in CHCl₃ (50 mL) and H₂O (150 mL) and were added in separating funnel. Next 2M H₃PO₄ (4.0 mL) was added and mixture shaken vigorously (pH z 2.0). The organic layer was washed with H₂O (150 mL) containing 2M H₃PO₄ (1.0 mL) and then carefully separated. The CHCl₃ was removed by azetrope distillation using acetonitrile in in vacuo. Precipitation from the resulting acetonitrile solution (100 mL) using H₂O gave ZnTPP-=CNCOOH (269 mg, 75%) as a purple powder.

¹H NMR (400 MHz, d₆-DMSO, TMS): δ 7.74-7.85 (m, 12H, H_{m.p.-Ph}), 8.07-8.11 (m, 3H, 2H_{o-Ph}+1H₃), 8.15-8.21 (m, 6H, H_{o-Ph}), 8.759 and 8.800 (ABq, 2H, ³J=4.7, 4.7 Hz, H_{β-pyrrolic}), 8.740 and 8.748 (ABq, 2H, ³J=4.7, 4.7 Hz, H_{β-pyrrolic}), 8.881 and 8.900 (ABq, 2H, ³J-4.7, 4.7 Hz, H_{β-pyrrolic}), 9.492 (d, 1H, ⁴J=1.0 Hz, H_{3′(β-pyrrolic)}), 13.470 (br s, 1H, CO₂H). Assignments aided by COSY spectra

UV-vis (THF): λₘₐₓ [nm] (ε×10⁻³) 407 (42.5), sh 429 (92.4), 455 (153), 571 (12.7), 620 (11.9).

FAB-LRMS: m/z (%, assignment) cluster at 771-778, 771 (100, M⁺). HRMS: Calcd for M⁺ (C₄₈H₂₉N₅O₂Zn): 771.1613, found: 771.1637.

### TPP-=CNCOOH (Piperidine Salt)

### 2-Cyano-3-(5′,10′,15′,20′-tetraphenylporphyrin-2′-yl)-acrylic acid piperidine salt

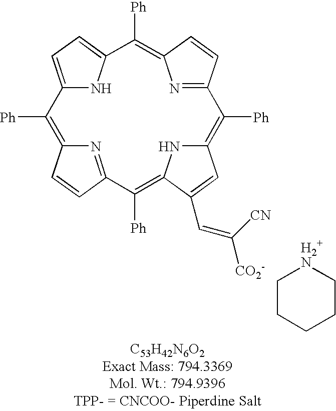

A solution of TPP-CHO (300 mg, 467 μmol), cyanoacetic acid (236 mg, 2.77 mmol, 6.0 eq) and piperidine (861 μL, 7.68 mmol, 19 eq) in acetonitrile (66 mL) was heated at reflux temperature for 3 h under N₂. On cooling to RT the resulting precipitate was collected by filtration (#4) and rinsed with acetonitrile to give TPP-=CNCOOH (Piperidine Salt) (367.3 mg, 99%) as a purple solid.

¹H NMR (400 MHz, CDCl₃, TMS): δ-2.607 (br s, 2H, NH), 1.75-1.81 (m, 2H, CH_{2, Piperidine}), 1.95-2.01 (m, 4H, CH_{2, Piperidine}), 3.257 (t, 4H, ³J=5.6 Hz, CH_{2, Piperidine}), 7.72-7.85 (m, 12H, H_{m.p.-Ph}), 8.044 (d, 1H, ⁴J=1.0 Hz H₃), 8.16-8.23 (m, 6H, H_{o-Ph}), 8.29-8.32 (m, 2H, H_{o-Ph}), 8.776 and 8.789 (ABq, 2H, ³J=4.8, 4.7 Hz, H_{β-pyrrolic}), 8.801 and 8.837 (ABq, 2H, ³J=4.9, 4.8 Hz, H_{β-pyrrolic}), 8.882 and 8.946 (ABq, 2H, ³J=4.9, 4.9 Hz, H_{β-pyrrolic}), 9.435 (d, 1H, ⁴J=0.9 Hz, H_{3′(β-pyrrolic)}), 10.0 (br s, 1H, H⁺_{Piperidine}). Assignments aided by COSY spectra.

UV-vis (THF): λₘₐₓ [nm] (ε×10⁻³) 435 (168), 526 (18.1), 568 (6.21), 608 (5.66), 667 (5.63).

FAB-LRMS: m/z (%, assignment) 86 (100, piperidine⁺), cluster at 771-778, 771 (45, M⁺).

### TPP-=CNCOOH.

### 2-Cyano-3-(5′,10′,15′,20′-tetraphenylporphyrin-2′-yl)-acrylic acid

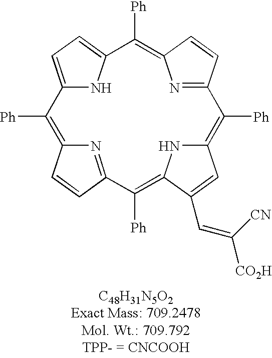

TPP-=CNCOOH (Piperidine Salt) (200 mg, 252 μmol) was dissolved in CHCl₃ (75 mL). H₂O (150 mL) was added in separating funnel. Next 2M H₃PO₄ (2.0 mL) was added and mixture shaken vigorously (pH≈2.0). The organic layer was washed with H₂O (150 mL) containing 2M H₃PO₄ (1.0 mL) and then carefully separated. The product was precipitated by removing the CHCl₃ with azetrope distillation using acetonitrile in vacuo to give TPP-=CNCOOH (166.2 mg, 93%) as a dark green powder.

¹H NMR (400 MHz, d₆-DMSO, 50° C., TMS): δ-2.649 (br s, 2H, NH), 7.78-7.89 (m, 12H, H_{m.p.-Ph}), 7.994 (s, 1H, H₃), 8.11-8.13 (m, 2H, H_{o-Ph}), 8.19-8.26 (m, 6H, H_{o-Ph}), 8.68-8.74 (m, 2H, H_{β-pyrrolic}), 8.80-8.95 (m, 4H, H_{β-pyrrolic}), 9.391 (s, 1H, H_{3′(β-pyrrolic)}). Assignments aided by COSY spectra.

UV-vis (THF): λₘₐₓ [nm] (ε×10⁻³) 436 (166), 526 (17.7), 568 (6.00), 606 (5.40), 666 (5.50).

FAB-LRMS: m/z (%, assignment) cluster at 709-712, 710 (100, MH⁺). HRMS: Calcd for M⁺ (C₄₈H₃₁N₅O₂): 710.2556, found: 710.2538.

### ZnTXP-CHO

### 2-Formyl-5,10,15,20-tetrakis(3′,5′-dimethylphenyl)porphyrinato zinc(II)

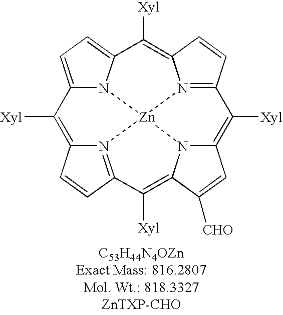

A solution of Zn(OAc)₂.2H₂O (105 mg, 478 μmol, 1.2 eq) in MeOH (3.0 mL) was added to a solution of TXP-CHO (300 mg, 397 μmol) in CHCl₃ (20 mL) with stirring at RT. After 20 min, TLC analysis indicated that the reaction was complete with the appearance of a new more polar green band (R_{f}=0.25 silica, 2:1 (CH₂Cl₂:hexane). The solvent was removed in vacuo and the residue was column chromatographed (silica, 37 mm^{dia}×130 mm, CH₂Cl₂:hexane (2:1)) collecting the major green/blue band. Recrystallisation from CH₂Cl₂/MeOH gave ZnTXP-CHO (236 mg, 73%) as a pink solid.

¹H NMR (400 MHz, CDCl₃, TMS): δ 2.57-2.61 (m, 24H, H_{Me-Xyl}), 7.403 (br s, 3H, H_{p-Xyl}), 7.432 (br s, 1H, H_{p-Xyl}), 7.778 (br s, 2H, H_{o-Xyl}), 7.808 (br s, 2H, H_{o-Xyl}), 7.818 (br s, 2H, H_{o-Xyl}), 7.862 (br s, 1H, H_{o-Xyl}), 8.91-8.95 (m, 5H, H_{β-pyrrolic}) 9.001 (d, −1H, ³J=4.7 Hz, H_{β-pyrrolic}), 9.296 (s, CHO), 9.553 (s, 1H, H_{3′(β-pyrrolic)}). Assignments aided by HETCOR spectra.

UV-vis (CH₂Cl₂): λₘₐₓ [nm] (ε×10⁻³) 311 (19.1), 435 (348), 527 (3.51), 561 (15.1), 604 (10.8), 685 (2.89).

FAB-LRMS: m/z (%, assignment) cluster at 816-822, 816 (100, M⁺). HRMS: Calcd for M⁺ (C₅₃H₄OZn): 816.2807, found: 816.2800.

### CuTPP-=CNCOOMe

### 2-Cyano-3-(2′-(5′, 10′,15′,20′-tetraphenylporphyrinato copper(II))yl)-acrylic acid methyl ester

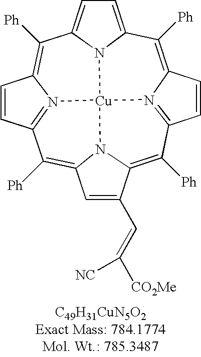

A mixture of CuTPP-CHO (100 mg, 142 μmol), methyl cyanoacetate (0.752 mL, 8.52 mmol, 60 eq) and piperidine (91 μL, 0.92 mmol, 6.5 eq) were irradiated with μ-wave for 3 min at 50% power (≈600 W). On cooling to RT CH₂Cl₂ (50 mL) was added and the solution filtered through paper (#1). The product was precipitated from solution with methanol to give CuTPP-=CNCOOMe (96.0 mg, 86%) as a dark green powder.

UV-vis (CH₂C₂): λₘₐₓ [nm] (ε×10⁻³) 308 (20.3). 388 (38.9), 444 (144), 556 (12.7), 603 (14.1).

FAB-LRMS: m/z (%, assignment) cluster at 784-788, 784 (100, M⁺). HRMS: Calcd for M⁺ (C₄₉H₃₁CuN₅O₂): 784.1774, found: 784.1759.

### CuTPP-=CNCOOH

### 2-Cyano-3-(2′-(5′,10′,15′,20′-tetraphenylporphyrinato copper(II))yl)-acrylic acid

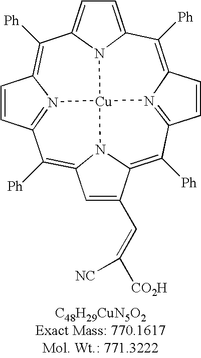

A mixture of CuTPP-CHO (210 mg, 298 μmol) and methyl cyanoacetate (1.87 mL, 21.2 mmol, 71 eq) in excess piperidine (22 mL) was irradiated with μ-wave for 2 min at 50% power (≈600 W). On cooling to RT the crude salt was precipitated with acetonitrile (150 mL). The resulting solid was dissolved in CHCl₃ (100 mL) and H₂O (100 mL) was added in separating funnel. Next 2M H₃PO₄ (3.0 mL) was added and mixture shaken vigorously (pH ≈2.0). The organic layer was washed with H₂O (100 mL) containing 2M H₃PO₄ (0.5 mL). The organic layer was then carefully separated, and the CHCl₃ was removed by azetrope distillation using acetonitrile in vacuo. Precipitation from the resulting acetonitrile solution gave CuTPP-=CNCOOH (175.4 mg, 76%) as a purple powder.

UV-vis (CH₂Cl₂): λₘₐₓ [nm] (ε×10⁻³) 309 (20.7), sh 391 (30.7), 439 (164), 555 (14.3), 601 (12.1).

FAB-LRMS: m/z (%, assignment) cluster at 770-774, 770 (100, M⁺). HRMS: Calcd for M⁺ (C₄₈H₂₉CuN₅O₂): 770.1672, found: 770.1630. ZnTXP-=CNCOOH

### 2-Cyano-3-(2′-(5′,10′,15′,20′-tetrakis(3′,5′-dimethylphenyl)porphyrinato zinc(II))yl)-acrylic acid

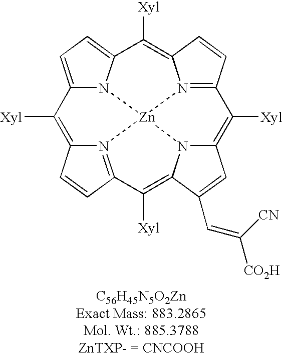

A solution of ZnTXP-CHO (200 mg, 244 μmol), cyanoacetic acid (92.0 mg, 1.08 mmol, 4.4 eq) and piperidine (451 μL, 4.56 mmol, 19 eq) in acetonitrile (35 mL) was heated at reflux temperature for 30 min under N₂. On cooling to RT CH₂Cl₂ (100 mL) and H₂O (200 mL) were added in separating funnel. Next 2M H₃PO₄ (4.0 mL) was added and mixture shaken vigorously (pH≈2.0). Additional CH₂Cl₂ (100 mL) was added and the organic layer was washed with H₂O (200 mL×3) containing 2M H₃PO₄ (1.0 mL). The organic layer was then carefully separated, and the CH₂Cl₂ was removed by azetrope distillation using acetonitrile in vacuo. Precipitation from the resulting acetonitrile solution (≈20 mL) using H₂O gave ZnTXP-=CNCOOH (176.2 mg, 81%) as a purple solid.

¹H NMR (400 MHz, d₆-DMSO, TMS): δ 2.52-2.59 (m, 24H, H_{Me-Xyl}), 7.40 (br s, 1H, H_{p-Xyl}), 7.42-7.44 (m, 3H, H_{p-Xyl}), 7.706 (br s, 2H, H_{o-Xyl}), 7.782 (br s, 4H, H_{o-Xyl}), 7.830 (br s, 2H, H_{o-Xyl}), 8.048 (d, 1H, ⁴J=0.7 Hz, H₃), 8.75-8.81 (m, 5H, H_{β-pyrrolic}), 8.876 (d, 1H, ³J=4.6 Hz, H_{β-pyrrolic}), 9.470 (d, 1H, ⁴J=0.9 Hz, H_{3′(β-pyrrolic)}). Assignments aided by COSY spectra.

UV-vis (THF): λₘₐₓ [nm] (ε×10⁻³) 319 (21.5), 410 (47.0), 458 (156), 573 (12.6), 623 (12.0).

FAB-LRMS: m/z (%, assignment) cluster at 883-889, 883 (100, M⁺). HRMS: Calcd for M⁺ (C₅₆H₄₅N₅O₂Zn): 883.2865, found: 883.2846.

### ZnTPP-=-CHO.

### 3-(2′-(5′,10′,15′,20′-tetraphenylporphyrinato zinc(II))yl)-allylaldehyde

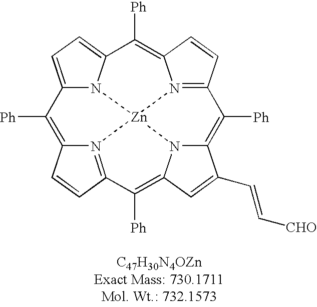

A solution of Zn(OAc)₂.2H₂O (79 mg, 359 μmol, 1.2 eq) in MeOH (1.5 mL) was added to a solution of TPP-=-CHO (200 mg, 299 μmol) as in CHCl₃ (20 mL) with stirring at RT. After 10 min, TLC analysis indicated that the reaction was complete with the appearance of a new more polar green band (R_{f}=0.38, silica, CH₂Cl₂). Precipitation from methanol gave ZnTPP-=-CHO (219 mg, 100%) as a purple microcrystalline powder.

¹H NMR (400 MHz, CDCl₃, TMS): δ 6.632 (dd, 1H, ³J=15.4 Hz, ³J=8.0 Hz, H_{2 (ethenyl)}), 6.963 (dd, 1H, ³J=15.6 Hz, ⁴J=0.7 Hz, H_{3 (ethenyl)}), 7.67-7.82 (m, 12H, H_{m.p.-Ph}), 8.11-8.13 (m, 2H, H_{o-Ph}), 8.07-8.09 (m, 6H, H_{o-Ph}), 8.85-8.92 (m, 7H, 6H_{β-pyrrolic}+1H_{CHO}), 9.208 (d, 1H, ⁴J=0.7 Hz, H_{3(β-pyrrolic)}). Assignments aided by COSY spectra.

UV-vis (CH₂Cl₂): λₘₐₓ [nm] (ε×10⁻³) 437 (255), sh 522 (4.53), 560 (18.3), 601 (10.9).

FAB-LRMS: m/z (%, assignment) cluster at 730-737, 730 (95, M⁺). HRMS: Calcd for M⁺ (C₄₇H₃₀N₄OZn): 730.1711 found: 730.1707.

### ZnTPP-=-=CNCOOH

### 2-Cyano-5-(2′-(5′, 10′, 15′,20′-tetraphenylporphyrinato zinc(II))yl)-penta-2,4-dienoic acid

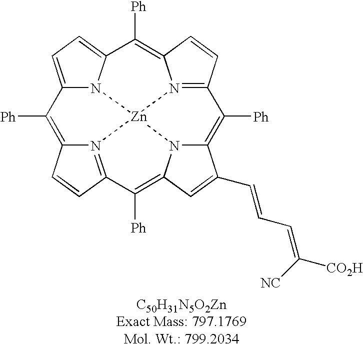

A solution of ZnTPP-=-CHO (150 mg, 205 μmol), cyanoacetic acid (87 mg, 1.02 mmol, 5.0 eq) and piperidine (490 μL, 5.0 mmol, 24 eq) in methanol (15 mL) was heated at reflux temperature for 50 min under N₂. On cooling to RT, CH₂Cl₂ (≈50 mL) and H₂O (≈100 mL) were added and shaken vigorously, adjusting the pH of the aqueous layer to pH=2 with 2M H₃PO₄ (3.0 mL). The organic layer was then carefully separated and the CH₂Cl₂ removed by azeotrope distillation using acetonitrile in vacuo. The crude product was precipitated from the resulting acetonitrile solution using H₂O and collected by filtration (#4). The solid was dissolved in CHCl₃ (aided by a little THF), filtered (#4) and the product precipitated with hexane to give ZnTPP-=-=CNCOOH (137 mg, 84%) as a dark green solid.

¹H NMR (400 MHz, d₆-DMSO, TMS): δ 6.743 (d, 1H, ³J=14.7 Hz, H_{5(pentadienyl)}), 7.203 (dd, 1H, ³J=14.7, 11.7 Hz, H_{4(pentadienyl)}), 7.388 (d, 1H, ³J=11.7 Hz, H_{3 (pentadienyl)}), 7.76-7.91 (m, 12H, H_{m,p-Ph}), 8.07-8.23 (m, 8H, H_{o-Ph}), 8.72-8.75 (m, 6H, H_{β-pyrrolic}) 9.060 (s, 1H, H_{3′(β-pyrrolic)}). Assignments aided by COSY spectra.

UV-vis (THF): λₘₐₓ [nm] (ε×10⁻³) 334 (28.4), 414 (73.8), sh 444 (109), 466 (121), 572 (16.5), 622 (18.5). UV-vis (DCM): λₘₐₓ [nm] (ε×10⁻³) 335 (22.3), 406 (68.7), 441 (85.9), 465 (96.3), 565 (13.8), 617 (19.3).

UV-vis (DMF): λₘₐₓ [nm] (ε×10⁻³) 317 (25.3), 444 (176), sh 535 (4.33), 571 (18.1), 612 (10.3).

FAB-LRMS: m/z (%, assignment) cluster at 797-803, 797 (100, M⁺). HRMS: Calcd for M⁺ (C₅₀H₃₁N₅O₂Zn): 797.1769, found: 797.1767.

Broad Soret band with multiple peaks.

### ZnTPP-=-PhCHO

### 4-(Trans-2′-(2″-(5″, 10″,15″,20″-tetraphenylporphyrinato zinc(II))yl)ethen-1′-yl)-1-benzaldehyde

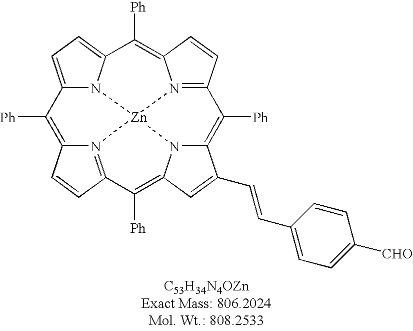

A solution of Zn(OAc)₂.2H₂O (71 mg, 322 μmol, 1.2 eq) in MeOH (2.0 mL) was added to a solution of TPP-=-Ph-CHO (200 mg, 269 μmol) in CHCl₃ (10 mL) with stirring at RT. After 20 min, TLC analysis indicated that the reaction was complete. Precipitation with methanol gave ZnTPP-=-PhCHO (218 mg, 100%) as a purple powder.

¹H NMR (400 MHz, CDCl₃, TMS): δ 7.177 and 7.256 (ABq, 2H, ³J=16.0, 16.0 Hz, H_{1′,2′}), 7.336 (d, 2H, ³J=8.0 Hz, H_{3,5}), 7.771 (m, 14H, 12H_{m.p-Ph}+2H_{2,6}), 8.217 (m, 8H, H_{o-Ph}), 8.815 and 8.894 (ABq, 2H, ³J=4.8, 4.8 Hz, H_{β-pyrrolic}), 8.903 and 8.935 (ABq, 2H, ³J=4.8, 4.8 Hz, H_{β-pyrrolic}), 8.913 (s, 2H, H_{β-pyrrolic}), 9.134 (d, 1H, ⁴J=0.8 Hz, H_{3″}), 9.918 (s, 1H, CHO).

UV-vis (CH₂Cl₂): λₘₐₓ [nm] (ε×10⁻³) 321 (28.1), 364 (24.3), 433 (231), 525 (5.54), 558 (24.8), 595 (12.7).

FAB-LRMS: m/z (%, assignment) cluster at 806-813, 806 (100, M⁺). HRMS: Calcd for M⁺ (C₅₃H₃₄N₄OZn): 806.2024, found: 806.2002.

### ZnTPP-=-Ph-=CNCOOH

### 2-Cyano-3-[4′-(trans-2″-(2′″-(5′″,10′″,15′″,20′″-tetraphenylporphyrinato zinc(II))yl)ethen-1″-yl)-phenyl]-acrylic acid

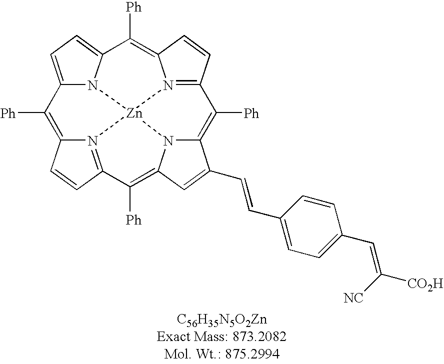

A solution of ZnTPP-=-PhCHO (150 mg, 186 μmol), cyanoacetic acid (313 mg, 3.68 mmol, 20 eq) and piperidine (1.02 mL, 10.3 mmol, 55 eq) in methanol (15 mL) was heated at reflux temperature for 15 h under argon. On cooling to RT, CH₂Cl₂ (≈100 mL) and H₂O (200 mL) were added and shaken vigorously, adjusting the pH of the aqueous layer to pH=2 with 2M H₃PO₄ (7.0 mL). The organic layer was then washed a second time with H₂O (200 mL) containing of 2M H₃PO₄ (1.0 mL, pH=2). The organic layer was then carefully separated and the product was precipitated with acetonitrile, removing the CH₂Cl₂ by azeotrope distillation in vacuo to give ZnTPP-=-Ph-=CNCOOH (133.3 mg, 82%) as a dark purple powder.

¹H NMR (400 MHz, d₆-DMSO, TMS): δ 7.143 and 7.354 (ABq, 2H, ³J=15.9, 15.9 Hz, H_{1″,2″}), 7.415 (d, 2H, ³J=8.5 Hz, H_{3′,5′}), 7.75-7.89 (m, 12H, H_{m.p-Ph}), 8.012 (d, 2H, ³J=8.5 Hz, H_{2′,6′}), 8.15-8.23 (m, 8H, H_{o-Ph}), 8.285 (s, 1H, H_{3, acrylic}), 8.656 and 8.723 (ABq, 2H, ³J=4.7, 4.6 Hz, H_{β-Pyrrolic}), 8.722 and 8.760 (ABq, 2H, ³J=4.7, 4.7 Hz, H_{β-pyrrolic}), 8.738 (s, 2H, H_{β-pyrrolic}) 9.036 (s, 1H, H_{3′″}). Assignments aided by COSY & LR-COSY spectra.

UV-vis (THF): λₘₐₓ [nm] (ε×10⁻³) 362 (27.4), 437 (136), 568 (21.9), 608 (14.8).

FAB-LRMS: m/z (%, assignment) cluster at 871-881, X (873, M⁺). HRMS: Calcd for M⁺ (C₅₆H₃₅N₅O₂Zn): 873.2082, found: 873.2063.

### ZnTXP-=-CHO

### 3-(2′-(5′,10′,15′,20′-tetra(3″,5″-dimethylphenyl)porphyrinato zinc(II))yl)-allylaldehyde

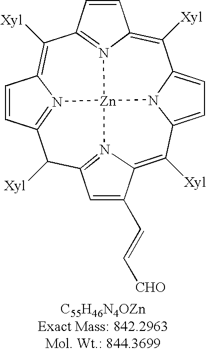

A solution of Zn(OAc)₂.2H₂O (85 mg, 388 μmol, 1.2 eq) in MeOH (2.0 mL) was added to a solution of TXP-=-CHO (250 mg, 320 μmol) as in CHCl₃ (25 mL) with stirring at RT. After 20 min, TLC analysis indicated that the reaction was complete with the appearance of a new more polar green band (R_{f}=0.13, silica, toluene). Precipitation from methanol gave ZnTXP-=-CHO (264 mg, 98%) as a purple microcrystalline powder.

¹H NMR (400 MHz, CDCl₃, TMS): δ 2.511 (s, 6H, H_{Me-Xyl}), 2.575 (s, 12H, H_{Me-Xyl}), 2.592 (s, 6H, H_{Me-Xyl}), 6.634 (dd, 1H, ³J=15.4 Hz, ³J=8.1 Hz, H_{2 (ethenyl)}), 7.020 (d, 1H, ³J=15.5 Hz, H_{3 (ethenyl)}), 7.394 (s, 2H, H_{o-Xyl}), 7.415 (s, 1H, H_{p-Xyl}), 7.430 (s, 1H, H_{p-Xyl}), 7.72 (br s, 2H, H_{o-Xyl}), 7.79 (br s, 2H, H_{o-Xyl}), 7.81 (br s, 4H, H_{o-Xyl}), 8.91-8.93 (m, 5H, H_{β-pyrrolic}), 8.958 (d, 1H, ³J=4.7 Hz, H_{β-pyrrolic}) 9.016 (d, 1H, ³J=8.0 Hz, CHO) 9.941 (d, 1H, ⁴J=0.6 Hz, H_{β-pyrrolic}). Assignments aided by COSY spectra

UV-vis (CH₂Cl₂): λₘₐₓ [nm] (ε×10⁻³) 439 (263), 561 (18.6), 603 (11.4).

FAB-LRMS: m/z (%, assignment) cluster at 842-848, 842 (100, M⁺). HRMS: Calcd for M⁺ (C₅₅H₄₆N₄OZn): 842.2963 found: 842.2946.

### ZnTXP-=-=CNCOOH

### 2-Cyano-5-(2′-(5′,10′,15′,20′-tetra(3″,5″-dimethylphenyl)porphyrinato zinc(II))yl)-penta-2,4-dienoic acid

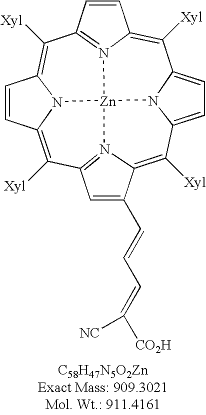

A solution of ZnTXP-=-CHO (150 mg, 178 μmol), cyanoacetic acid (75.6 mg, 889 μmol, 5.0 eq) and piperidine (422 μL, 4.27 mmol, 24 eq) in methanol (15 mL) was heated at reflux temperature for 2 h under N₂. On cooling to RT, CH₂Cl₂ (≈50 mL) and H₂O (≈100 mL) were added and shaken vigorously, adjusting the pH of the aqueous layer to pH=2 with 2M H₃PO₄ (3.5 mL). The organic layer was then carefully separated and the CH₂Cl₂ removed by azeotrope distillation using acetonitrile in vacuo. The product was precipitated from the resulting acetonitrile solution using H₂O and collected by filtration (#4) to give ZnTXP-=-=CNCOOH (157 mg, 97%) as a purple powder.

¹H NMR (400 MHz, d₆-DMSO, TMS): δ 2.540 (s, 6H, H_{Me-Xyl}), 2.575 (s, 12H, H_{Me-Xyl}), 2.592 (s, 6H, H_{Me-Xyl}), 6.459 (d, 1H, ³J=14.6 Hz, H_{5(pentadienyl)}), 7.207 (dd, 1H, ³J=14.8, 11.7 Hz, H_{4 (pentadienyl)}), 7.42-7.53 (m, 5H, 4H_{p-Xyl}+1H₃), 7.718 (s, 2H, H_{o-Xyl}), 7.773 (s, 4H, H_{o-Xyl}), 7.824 (s, 2H, H_{o-Xyl}), 8.75-8.78 (m, 5H, H_{β-pyrrolic}), 8.812 (d, 1H, ³J=4.6 Hz, H_{β-pyrrolic}), 9.055 (s, 1H, H_{3′(β-pyrrolic)}). Assignments aided by COSY & LR-COSY spectra.

UV-vis (THF): λₘₐₓ [nm] (ε×10⁻³) 334 (28.9), 417 (83.5), 430 (98.4), sh 447 (103), 469 (116), 573 (16.7), 623 (17.8).

FAB-LRMS: m/z (%, assignment) cluster at 909-915, 909 (100, M⁺). HRMS: Calcd for M⁺ (C₅₈H₄₇N₅O₂Zn): 909.3021, found: 909.3014.

### CuTBP-CHO

### 2-Formyl-5,10,15,20-tetra-n-butylporphyrinato copper(II)yl

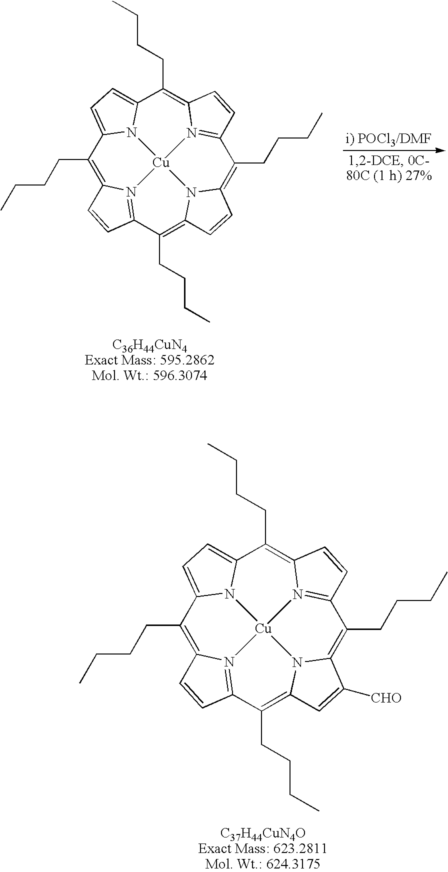

A Vilsmeier complex was prepared by adding POCl₃ (13.5 mL, mol) slowly over 10 min to dry DMF (14.6 mL, 2.01 mol) at 0° C. under argon in 500 mL 3 neck round-bottom flask. After 25 min a solution of Cu-TBP (1.15 g, 1.93 mmol) in dry 1,2-DCE (170 ml) was added and the reaction heated at 80° C. for 1 h. On cooling to RT mixture was poured into ice cold RO water (1.5 L) and extracted into CH₂Cl₂ (2×1 L). The aqueous layer was decanted off and the organic layer washed with H₂O (3×1.5 L) then sat. aq. NaHCO₃ (500 ml). The organic layer was separated then dried (MgSO₄), filtered and the solvent removed in vacuo. The residue was column chromatographed (silica, 70 mm^{dia}×200 mm) first eluting with CH₂Cl₂:Hexane (1:1) to give starting material (162 mg, 14%, recrystallized from CH₂Cl₂/MeOH), then CH₂Cl₂ to give the title compound (333 mg, 27%, recrystallized from CH₂Cl₂/MeOH) as a purple solid. UV-vis (CHCl₂): λₘₐₓ [nm] (ε×10⁻³) 309 (13.6), 434 (181), 561 (9.13), 608 (7.88). FAB-HRMS: m/z (%, assignment) cluster at 621-627, 623 (100, M⁺). HRMS: Calcd for M⁺ (C₃₇H₄₄CuN₄O): 623.2811, found: 623.2819.

### TBP-CHO

### 2-Formyl-5,10,15,20-tetra-n-butylporphyrin

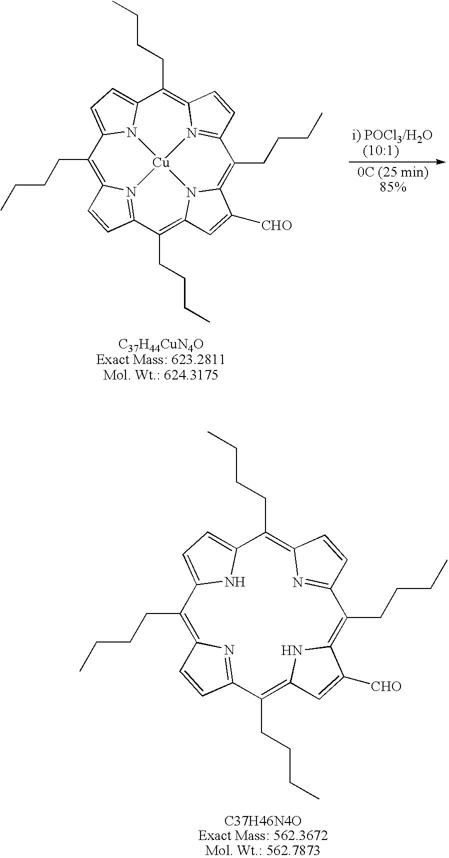

H₂O (1.5 ml) was added to a stirring solution of Cu-TBP in POCl₃ (15 ml) at 0° C. After stirring for 25 min at 0° C. the mixture was poured into stirring ice cold H₂O (400 ml) and 25% Ammonia Solution (˜80 ml) was added until pH>7 forming a purple precipitate. The precipitate was collected by filtration, redisolved in CH₂Cl₂ and the solvent removed in vacuo. The residue was column chromatographed (silica, 50 mm_{dia}×70 mm, CH₂Cl₂) to give the title compound (162 mg, 85%, recrystallized from CH₂Cl₂/MeOH) as a purple solid. ¹H NMR (500 MHz, CDCl₃) δ-2.315 (br s, 2H, NH), 1.08-1.15 (m, 12H, CH_{3 Butyl}), 1.71-1.84 (m, 8H, CH_{2 Butyl}), 2.38-2.49 (m, 8H, CH_{2 Butyl}), 4.80-4.88 (m, 6H, CH_{2 Butyl}), 4.94-4.97 (m, 2H, CH₂ Butyl), 9.356 (s, 1H, H_{β-pyrrolic}) 9.394 and 9.456 (ABq, 2H, ³J=4.87 Hz, H_{β-pyrrolic}), 9.371 and 9.428 (ABq, 2H, ³J=4.90 Hz, H_{β-pyrrolic}), 9.960 (s, 1H, H_{3, βpyrrolic}), 11.379 (s, 1H, CHO). Assignments aided by COSY and HMQC. UV-vis (CHCl₂): λₘₐₓ [nm] (ε×10⁻³) 435 (145), 531 (9.12), 581 (4.04), 620 (2.96), 683 (6.29). FAB-HRMS: m/z (%, assignment) cluster at 560-565, 563 (100, MH⁺). HRMS: Calcd for MH⁺ (C₃₇H₄₇N₄O): 563.3749, found: 563.3747.

### ZnTBP-CHO

### 2-Formyl-5,10,15,20-tetra-n-butylporphyrinato zinc(II)yl

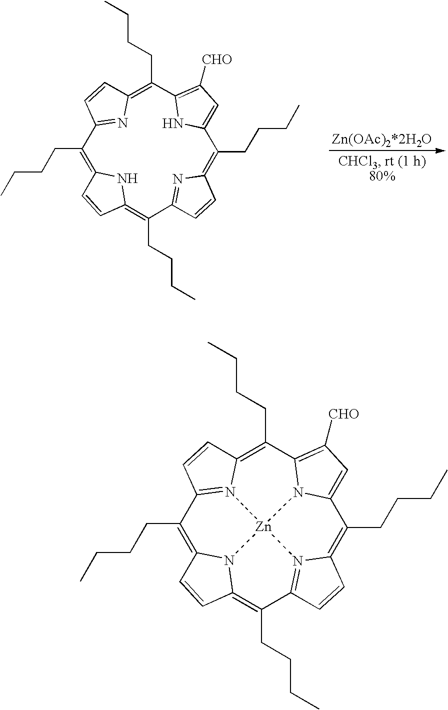

A solution of Zn(OAc)₂.2H₂O (75 mg, 341 μmol, 1.2 eq) in MeOH (1.5 mL) was added to a solution of TBP-CHO (128 mg, 227 μmol) as in chloroform (9 mL) with stirring at RT. After 60 min, TLC analysis indicated that the reaction was complete with the appearance of a new more polar green band (silica, CH₂Cl₂:hexane (2:1)). The solvents were remowed under vacuo and the remaining was recrystallized from chloroform/MeOH mixture to give the ZnTBP-=-CHO (114 mg, 80%) as a greenish powder. ¹H NMR (500 MHz, CDCl₃, TMS): δ 11.08 (s, 1H, CHO), 9.52 (s, 1H, H_{β-pyrrolic)}), 9.04 (d, 1H, J=4.5 Hz, H_{(β-pyrrolic)}), 9.01 (d, 1H, J=4.5 Hz, H_{(β-pyrrolic)}), 8.93 (d, 1H, J=4.8 Hz, H_{β-pyrrolic)}), 8.90 (d, 1H, J=4.8 Hz, H_{(β-pyrrolic)}), 8.81 (d, 1H, J=4.5 Hz, H_{(β-pyrrolic)}), 8.77 (d, 1H, J=4.5 Hz, H_{(β-pyrrolic)}), 4.43 (t, 2H, J=8.3 Hz, Porph-CH₂), 4.37 (t, 2H, j=8.3 Hz, Porph-CH₂), 4.30 (t, 1H, J=8.3 Hz, Porph-CH₂), 4.23 (t, 2H, J=8.3 Hz, Porph-CH₂), 2.33-2.23 (m, 8H, Alk-C2), 1.81-1.68 (m, 8H, Alk-C3), 1.14-1.07 (m, 12H, Alk-CH₃). UV-vis (CH₂Cl₂): λₘₐₓ [nm] (log ε) 314 (4.10), 437 (5.26), 570 (3.91), 619 (3.86). FAB-LRMS: m/z (%, assignment) cluster at 622-632, 624 (60, M⁺). HRMS: Calcd for M⁺ (C₃₇H₄₄N₄O₁Zn₁): 624.28066, found: 624.28035.

### ZnTBP-=(CN)COOH

### 2-Cyano-3-(2′-(5′,10′,15′,20′-tetra-n-butylporphyrinato zinc(II))yl)-acrylic acid

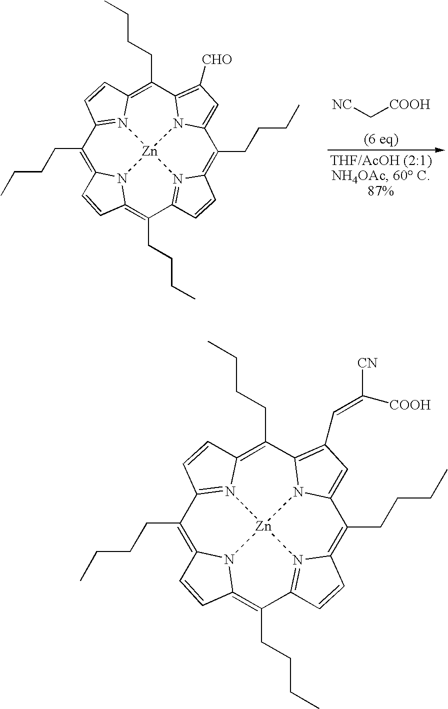

A solution of ZnTBP-CHO (50 mg, 80 μmol), cyanoacetic acid (68 mg, 0.8 mmol, 10 eq) and ammonium acetate (62 mg, 0.8 mmol, 10 eq) in a solution of acetic acid:THF (1:2, 6 mL) was heated at 60° C. for 4 hs. On cooling to room temperature H₂O (15 mL) was added, precipitating the crude product. After purification on silica (20% MeOH in DCM) and recrystalisation from acetone/hexane ZnTBP-=(CN)COOH (48 mg, 87%) as a dark solid was received. ¹H NMR (500 MHz, d₆-DMSO, TMS): δ 10.03 (br s, 1H, COOH), 9.72 (s, 1H, vinyl-H), 9.61-9.54 (m, 7H, H_{β-pyrrolic}), 4.95-4.81 (m, 8H, Porph-CH₂-Alk), 2.46-2.38 (m, 8H, Alk-C2), 1.94-1.85 (m, 8H, Alk-C3), 1.16-1.08 (m, 12H, Alk-CH₃). HRMS: Calcd for M⁺ (C₄₀H₄₆N₄O₄Zn): 710.28105, found: 710.27922.

### TBP-=-CO₂Me

### 3-trans-(5′,10′,15′,20′-tetra-n-butylporphyrin-2′-yl)-acrylic acid methyl ester

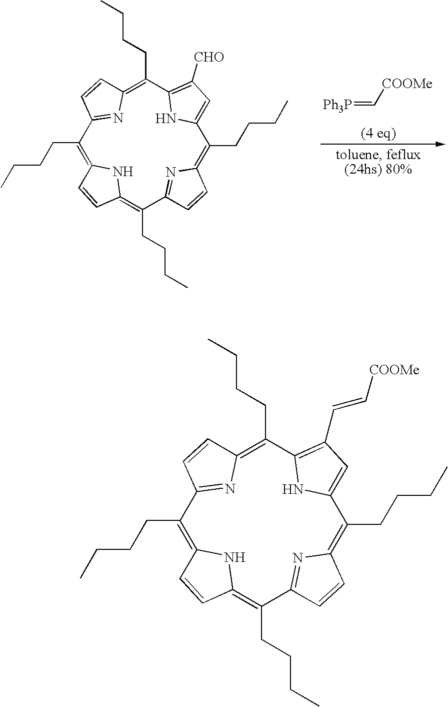

A solution of TBP-CHO (1.091 g, 1.94 mmol) and phosphorane Ph₃P=CHCO₂Me (2.754 g, 7.75 mmol, 4 eq) in dry toluene (100 mL) was heated at reflux temperature under Argon. After 24 hs, TLC analysis (silica, toluene) indicated that all of the starting material TBP-CHO had been consumed. After cooling to RT the solvent was removed in vacuo. The residue was column chromatographed (silica, 10% AcOEt in toluene) collecting the major purple colored fraction and recrystallized from DCM/MeOH to give a trans isomer of TBP-=-CO₂Me (0.959 g, 80%) as a purple solid. ¹H NMR (500 MHz, CDCl₃, TMS): 9.42-9.13 (m, 7H, H_{(β-pyrrolic)}), 9.15 (d, 1H, J=15.5 Hz, vinyl-H), 6.96 (d, 1H, J=15.5 Hz, vinyl-H), 4.91-4.83 (m, 8H, Porph-CH₂-Alk), 4.02 (s, 3H, COOMe), 2.53-2.44 (m, 8H. Alk-C2), 1.97-1.90 (m, 2H, Alk-C3), 1.85-1.80 (m, 6H, Alk-C3), 1.20 (t, 3H, J=7.5 Hz, Alk-CH₃), 1.16-1.12 (m, 9H, Alk-CH₃), −2.49 (br s, 2H, NH). UV-vis (CH₂Cl₂): Imax [nm] (log ε) 409 sh (4.72), 425 (5.14), 527 (3.96), 565 (3.64), 612 (3.42), 669 (3.55). FAB-LRMS: m/z (%, assignment) cluster at 618-619, 618 (95, M⁺). HRMS: Calcd for M⁺ (C₄₀H₅₀N₄O₂): 618.39338, found: 618.39338.

### TBP-=-CH₂OH

### 3-(5′,10′,15′, 20′-tetra-n-butylporphyrin-2′-yl)-allylhydroxide

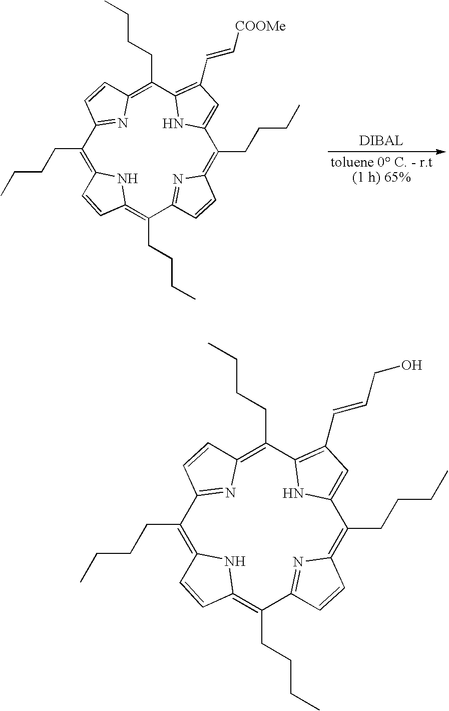

DIBAL-H (3.1 mL, 1.5 M in toluene, 4.60 mmol, 3.0 eq) was added to a solution of TBP-=-CO₂Me (0.948 g, 1.53 mmol) in dry toluene (50 mL) under argon atmosphere at 0° C. After 30 min the reaction was allowed to warm to RT. After another 30 min MeOH (5.0 mL) added followed by 10% NaOH (50 mL). CH₂Cl₂ (150 mL) was added and the organic layer separated, dried (MgSO₄) and the solvent removed in vacuo. The residue was column chromatographed (silica, toluene) collecting the first major red colored fraction. Recrystallization from CH₂Cl₂:hexane gave TBP-=-CH₂OH (587 mg, 65%) as a red powder. ¹H NMR (500 MHz, CDCl₃, TMS): 9.43-9.41 (m, 6H, H_{β-pyrrolic}), 9.34 (s, 1H, H_{β-pyrrolic}), 8.00 (d, 1H, J=15.4 Hz, vinyl-H), 6.83 (dt, 1H, J=5.5 and 15.4 Hz, vinyl-H), 4.92-4.87 (m, 8H, Porph-CH₂), 4.71 (br s, 1H, CH₂—OH), 2.55-2.40 (m, 8H, Alk-C2), 1.89-1.78 (m, 8H, Alk-C3), 1.20-1.12 (m, 12H, Alk-CH₃), −2.54 (br s, 2H, NH). UV-vis (CH₂Cl₂): λₘₐₓ [nm] (log E) 406 sh (4.86), 420 (5.44), 523 (4.10), 560 (3.87), 605 (3.57), 663 (3.71). FAB-LRMS: m/z (%, assignment) cluster at 587-590, 590 (45, M⁺). HRMS: Calcd for M⁺−1 (C₃₉H₄₉N₄O₁): 589.39064, found: 589.38856.

### TBP-=-CHO

### 3-(5′,10′,15′,20′-tetra-n-butylporphyrin-2′-yl)-allylaldehyde

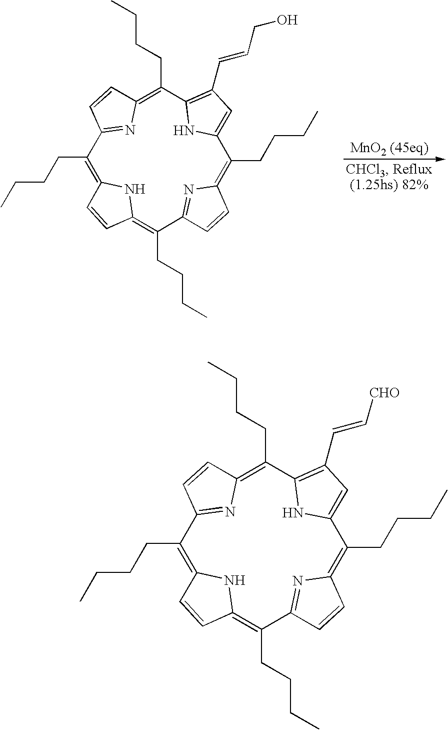

Activated MnO₂ (3.234 g, 0.04 mol, 45 eq) was added to a solution of TBP-=-CH₂OH (550 mg, 930 μmol) in dry CHCl₃ (20 mL) and heated at reflux temperature for 1.25 hs under argon atmosphere, TLC analysis (silica, CH₂Cl₂:hexane (2:1)) indicated all staring material had been consumed with the appearance of a single new less polar band. On cooling to room temperature, the solution was filtered through celite and the solvent removed in vacuo. The purple solid was purified on silica (DCM). Precipitation from CH₂Cl₂:methanol gave TBP-=-CHO (446 mg, 82%) as a purple powder. ¹H NMR (500 MHz, CDCl₃, TMS): δ 10.7 (d, 1H, J=7.6 Hz, CHO), 9.44-9.38 (m, 7H, H_{β-pyrrolic}), 8.85 (d, 1H, J=15.6 Hz, vinyl-H), 7.24 (dd, 1H, J=7.6 and 15.6 Hz, vinyl-H), 4.89-4.83 (m, 6H, Porph-CH₂), 4.77-4.74 (m, 2H, Porph-CH₂), 2.51-2.42 (m, Alk-C2), 1.85-1.77 (m, 8H, Alk-C3), 1.18-1.11 (m, 12H, Alk-CH₃), −2.43 (br s, 2H, NH). UV-vis (CH₂Cl₂): λₘₐₓ [nm] (log s) 359 (4.38), 410 sh (4.87), 427 (5.20), 530 (4.14), 573 (3.76), 615 (3.62), 674 (3.71). FAB-LRMS: m/z (%, assignment) cluster at 756-591, 588 (83 M⁺). HRMS: Calcd for M⁺ (C₃₉H₄₈N₄O₁): 588.38281, found: 588.38440.

### ZnTBP-=-CHO

### 3-(2′-(5′, 10′,15′,20′-tetra-n-butylporphyrinato zinc(II))yl)-allylaldehyde

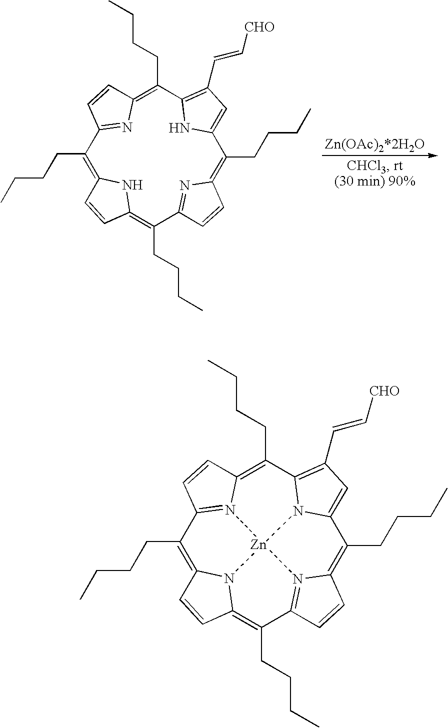

A solution of Zn(OAc)₂.2H₂O (188 mg, 856 μmol, 1.2 eq) in MeOH (2.0 mL) was added to a solution of TBP-=-CHO (420 mg, 713 μmol) as in chloroform (10 mL) with stirring at RT. After 30 min, TLC analysis indicated that the reaction was complete with the appearance of a new more polar green band (silica, CH₂Cl₂:hexane (2:1)). The solvents were remowed under vacuo and the remaining was recrystallized from DCM/MeOH mixture to give the ZnTBP-=-CHO (420 mg, 90%) as a greenish powder. ¹H NMR (500 MHz, CDCl₃, TMS): δ 10.0 (s, 1H, J=7.9 Hz, CHO), 9.22 (dd, 2H, J=3.6 and 4.6 Hz, H_{(β-pyrrolic)}), 9.17 (d, 1H, J=4.2 Hz, H_{(β-pyrrolic)}), 9.09 (d, 1H, J=4.2 Hz, H_{(β-pyrrolic)}) 9.04 (d, 1H, J=4.0 Hz, H_{(β-pyrrolic)}), 8.93-8.92 (m, 2H, H_{(β-pyrrolic)}), 8.42 (d, 1H, J=15.0 Hz, vinyl-H), 6.97 (dd, 1H, J=7.9 and 15.0 Hz, vinyl-H), 4.65-4.62 (m, 2H, Porph-CH₂), 4.59-4.56 (m, 2H, Porph-CH₂), 4.37-4.33 (m, 2H, Porph-CH₂), 4.15-4.13 (m, 2H, Porph-CH₂), 2.44-2.41 (m, 4H, Alk-C2), 2.28-2.21 (m, 4H, Alk-C2), 1.87-1.69 (m, 8H, Alk-C3), 1.18-1.09 (m, 12H, Alk-CH₃). UV-vis (CH₂Cl₂): λₘₐₓ [nm] (log c) 437 (5.21), 566 (4.11), 611 (3.92). FAB-LRMS: m/z (%, assignment) cluster at 648-658, 650 (85, M⁺). HRMS: Calcd for M⁺ (C₃₉H₄₆N₄O₁Zn₁): 650.29631, found: 650.29351.

### ZnTBP-=-=(CN)COOH

### 2-Cyano-5-(2′-(5′,10′,15′,20′-tetra-n-butylporphyrinato zinc(II))yl)-penta-2,4-dienoic acid

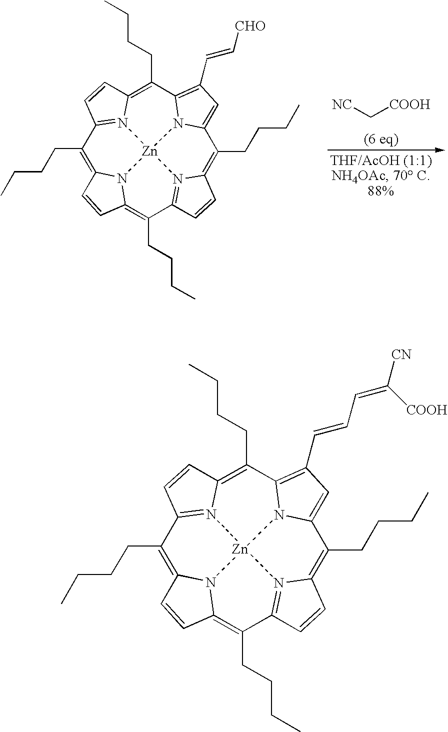

A solution of ZnTBP-=-CHO (150 mg, 230 μmol), cyanoacetic acid (117 mg, 1.38 mmol, 6.0 eq) and ammonium acetate (106 mg, 1.38 mmol, 6.0 eq) in a solution of acetic acid:THF (1:1, 14 mL) was heated at 70° C. for 1.5 hrs. On cooling to room temperature H₂O (25 mL) was added, precipitating the crude product. After purification on silica (5% MeOH, 1% AcOH in DCM) and recrystalisation from THF/acetonitrile ZnTBP-=-=(COOH)₂ (145 mg, 88%) as a dark greenish solid was received. ¹H NMR (500 MHz, d₆-DMSO, TMS): δ 13.56 (br s, 1H, COOH), 9.82 (s, 1H, H_{β-pyrrolic}), 9.60-9.54 (m, 6H, H_{β-pyrrolic}), 9.03 (d, 1H, J=14.6 Hz, vinyl-H), 8.48 (d, 1H, J=11.4 Hz, vinyl-H), 7.69 (dd, 1H, J=14.6 and 11.4 Hz, vinyl-H), 4.40-4.89 (m, 8H, Porph-CH₂-Alk), 2.41-2.36 (m, 8H, Alk-C2), 1.81-1.75 (m, 8H, Alk-C3), 1.36-1.09 (m, 12H, Alk-CH₃). FAB-LRMS: m/z (%, assignment) cluster at 716-722, 717 (56, M⁺). HRMS: Calcd for M⁺ (C₄₂H₄₇N₅O₂Zn): 717.30212, found: 717.30469.

### CuTMPP

### 5,10,15,20-Tetrakis(4′-methoxyphenyl)porphyrinato copper(II)

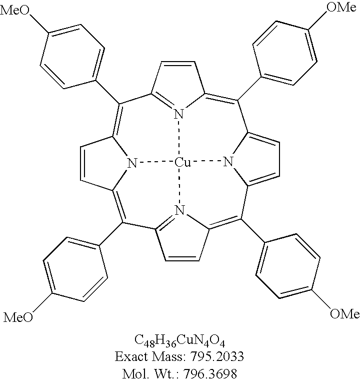

A solution of Cu(OAc)₂—H₂O (0.489 g, 2.44 mmol, 1.2 eq) in MeOH (40 mL) was added to a refluxing solution of TMPP (1.50 g, 2.04 mmol) in CHCl₃ (300 mL) with stirring under N₂ atmosphere. TLC analysis after 1 h indicated that all the starting material TMPP had been consumed with the appearance of a new red band of higher R_{f} (R_{f}=0.125, silica, CH₂Cl₂:hexane (1:1)). On cooling to RT the product was precipitated with MeOH to give ZnTMPP (1.52 g, 92%) as a purple microcrystalline solid.

UV-vis (CH₂Cl₂): λₘₐₓ [nm] (ε×10⁻³) 418 (435), 541 (18.7), 578 (4.52), 617 (2.70).

FAB-LRMS: m/z (%, assignment) cluster at 795-800, 795 (100, M⁺). HRMS: Calcd for M⁺ (C₄₈H₃₆CuN₄O₄): 795.2033, found: 795.2040.

### CuTMPP-CHO

### 2-Formyl-5,10,15,20-tetrakis(4′-methoxyphenyl)porphyrinato copper(II)

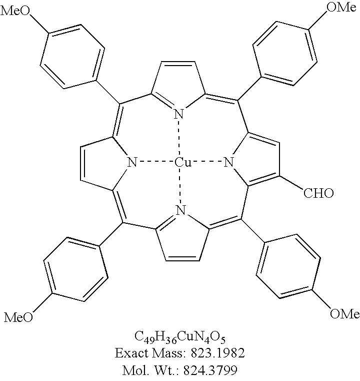

A Vilsmeier complex was prepared by adding POCl₃ (3.48 mL, 37.3 mol) slowly to dry DMF (4.37 mL, 56.4 mol) at 0° C. under argon. After 20 min, the viscous oil was warmed to RT. Dry 1,2-DCE (45 mL) and CuTMPP (446 g, 560 mmol) was added and the reaction heated at 90° C. for 1 hour under argon. On cooling to RT, CH₂Cl₂ (500 mL) and H₂O (1 L) was added. The organic layer was washed with H₂O (1 L×3), and then sat. aq. NaHCO₃ (500 mL). The organic layer was then separated and dried (K₂CO₃), filtered and the solvent removed in vacuo. Recrystallisation from CH₂Cl₂/MeOH gave CuTMPP-CHO (382.8 mg, 83%) as a purple powder.

UV-vis (CH₂Cl₂): λₘₐₓ [nm] (ε×10⁻³) 307 (14.7), 433 (227), sh 519 (3.66), 555 (11.7), 595 (8.48), 692 (1.81).

FAB-LRMS: m/z (%, assignment) cluster at 823-827, 823 (100, M⁺). HRMS: Calcd for M⁺ (C₄₉H₃₆CuN₄O₅): 823.1982, found: 823.1978.

### CuTMPP-=CNCOOH.

### 2-Cyano-3-(2′-(5′,10′,15′,20′-tetra(4″-methoxyphenyl)porphyrinato zinc(II))yl)-acrylic acid

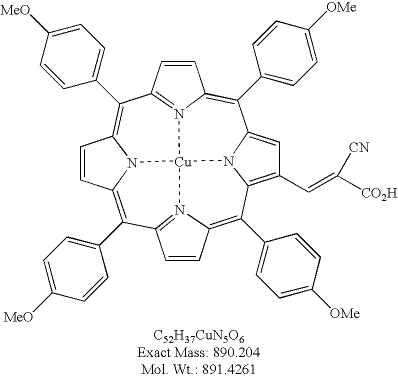

A solution of CuTMPP-CHO (250 mg, 303 μmol), cyanoacetic acid (77.4 mg, 909 μmol, 3.0 eq) and piperidine (560 μL, 5.66 mmol, 19 eq) in acetonitrile (43 mL) was heated at reflux temperature for 2 h under N₂. On cooling to RT the resulting red precipitate was collected by filtration (#4) and the solid was dissolved in a mixture of DMSO (3.0 mL) and CHCl₃ (≈80 mL). H₂O (100 mL) was added in a separating funnel followed by 2M H₃PO₄ (4.0 mL) and the mixture shaken vigorously (pH 2.0). The organic layer was washed with H₂O (250 mL×3) containing 2M H₃PO₄ (2.0 mL) and then carefully separated. The CHCl₃ was removed by azetrope distillation using acetonitrile in vacuo, precipitating the product to give CuTMPP-=CNCOOH (205.3 mg, 76%) as a purple powder.

UV-vis (THF): λₘₐₓ [nm] (ε×10⁻³) 307 (22.7), 433 (186), 556 (15.1), 600 (11.2).

FAB-LRMS: m/z (%, assignment) cluster at 890-894, 890 (100, M⁺). HRMS: Calcd for M⁺ (C₅₂H₃₇CuN₅O₆): 890.2040, found: 890.2048.

### ZnDPP-CHO

### 5-Formyl-10,20-diphenylporphyrinato zinc(II)yl

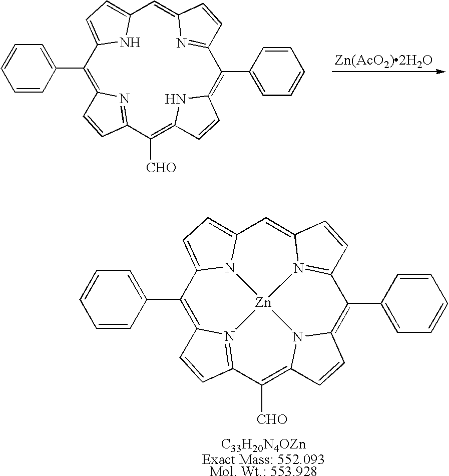

A solution of Zn(AcO)₂.2H₂O in 3.5 ml of MeOH was added to a stirring room temperature solution of DPP-CHO in 355 ml of CHCl₃ and stirred until the reaction had gone to completion by TLC (2 hr). The solution was taken to dryness in vacuo (50 degrees), the solid redissolved in a mixture of 700 ml CHCl₃, 5 ml THF and 7 drops triethylamine and passed down a plug of Silica (50 mm×60 mm) to remove excess zinc aceatate. Recrystallization from acetonitrile and collected by vacuum filtration (#4) yielded ZnDPP-CHO as a purple solid (218 mg, 97%). ¹H NMR (400 MHz, DMSO d-6, TMS): δ 12.56 (s, 1H, CHO), 10.44 (s, 1H, Hₘₑₛₒ), 10.19 and 8.76 (ABq, 4H, 3J=4.84 Hz, H_{β}), 9.45 and 8.93 (ABq, 4H, 3J=4.36 Hz, H_{β}), 8.19 (dd, 4H, Ph_{H-ortho}), 7.84 (m, 6H, Ph_{H-meta,para}). UV-vis (DMF); λₘₐₓ [nm] (ε×103) 320 (14.21), 427 (255), 524 (2.69), 561 (10.2), 602 (12.2). FAB-LRMS: m/z (%, assignment) cluster at 551-558, 825 (100, M⁺). HRMS: Calcd for M⁺ (C₃₃H₂₀N₄OZn): 552.0928, found: 552.0902.

### ZnDPP-=CNCOOH

### 2-Cyano-3-(10′,20′-diphenylporphyrinato zinc(II)yl)-acrylic acid

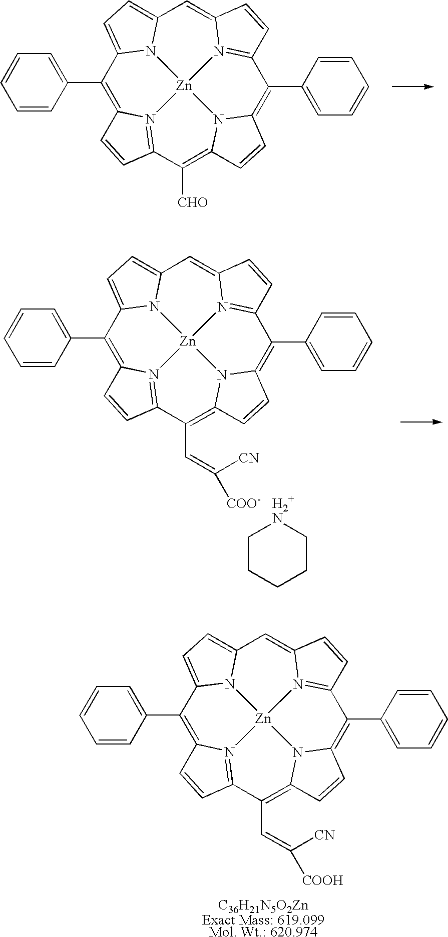

A solution of ZnDPP-CHO (101 mg, 182 μmol), cyanoacetic acid (93.1 mg, 1092 μmol, 6 eq) and piperidine (342 μl, 3458 μmol) in actonitrile (25 ml) was heated to reflux (100 degrees) for 3 hrs 40 min under nitrogen. Cooled to room temperature and a mixture of 2 ml THF and 25 ml CHCl₃ was added to ensure solubility during separation. This was added to a separation funnel along with 100 ml of water and enough 2M H₃PO₄ (2.5 ml) to bring the organic layer to pH approx. 2.0 and shaken vigorously. The organic layer was then washed with 75 ml of water containing 0.5 ml of 2 M H₃PO₄ and carefully separated. The organic layer was then passed down a Silica column (42 mm×70 mm) with DCM until all the solvent front material had been eluted ZnDPP-CHO then the elutent changed to a mixture of DCM, 5% MeOH and 0.5% AcOH and the base line material was eluted. The solvent was removed in vacuo (53 degrees) and the solid redissolved in acetonitrile (approx. 15 ml) with enough THF to allow complete solubility of the porphyrin (approx. 5 ml). Enough water was added to cause precipitation, the porphyrin was collect by vacuum filtration (#4) and washed with 1:2 acetonitrile:water giving an off purple solid (76 mg, 66%). ¹H NMR (400 MHz, DMSO d-6, TMS): δ 10.88 (br s, 1H, —CH═CNCOOH), 10.40 (s, 1H, Hₘₑₛₒ), 9.51 and 8.90 (ABq, 4H, 3J=4.68 Hz, H_{β}), 9.48 and 8.84 (ABq, 4H, 3J=4.44 Hz, H_{β}), 8.21 (m, 4H, Ph_{H-ortho}), 7.84 (m, 6H, Ph_{H-meta,para}). UV-vis (DMF); λₘₐₓ [nm] (ε×10³) 314 (14.71), 351 (9.31), 424 (279), 519 (2.40), 557 (12.7), 600 (4.46). FAB-LRMS: m/z (%, assignment) cluster at 619-624, 619 (100, M⁺). HRMS: Calcd for M⁺ (C₃₆H₂₁N₅O₂Zn): 619.0986, found: 619.0976.

### Porphyrin Malonic Acids

### ZnTBP-=(COOH)₂

### 2-Carboxy-3-(2′-(5′, 10′,15′,20′-tetra-n-butylporphyrinato zinc(II))yl)-prop-2-enoic acid

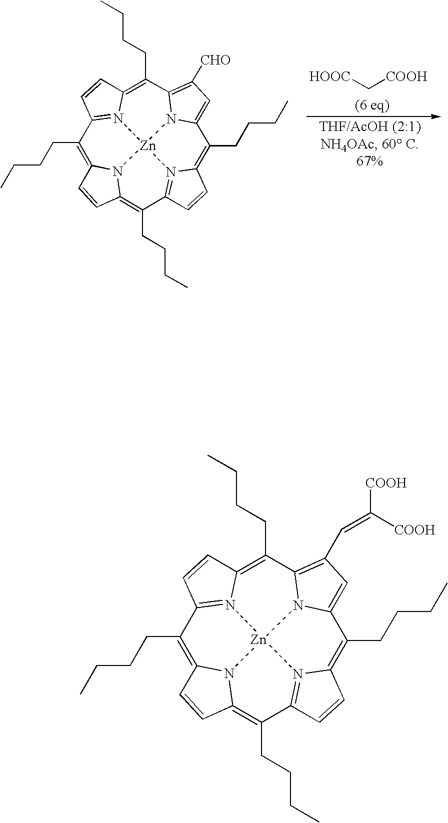

A solution of ZnTBP-CHO (50 mg, 80 μmol), malonic acid (83 mg, 0.8 mmol, 10 eq) and ammonium acetate (62 mg, 0.8 mmol, 10 eq) in a solution of acetic acid:THF (1:2, 6 mL) was heated at 60° C. for 4.5 hs. On cooling to room temperature H₂O (15 mL) was added, precipitating the crude product. After purification on silica (30% MeOH in DCM) and recrystalisation from THF/MeOH/hexane ZnTBP-=(COOH)₂ (38 mg, 67%) as a green solid was received. ¹H NMR (500 MHz, d₆-DMSO, TMS): δ 13.18 (br s, 1H, COOH), 9.58-9.56 (m, 6H, H_{β-pyrrolic}) 9.39 (s, 1H, H_{β-pyrrolic}), 9.25 (br s, 1H, COOH), 4.95-4.23 (m, 8H, Porph-CH₂-Alk), 2.43-2.40 (m, 8H, Alk-C2), 1.83-1.74 (m, 8H, Alk-C3), 1.13-1.11 (m, 12H, Alk-CH₃). UV-vis (DMF): λₘₐₓ [nm] (log ε) 314 (4.22), 413 sh (4.67), 429 (5.43), 531 (3.52), 570 (4.07), 610 (3.91). FAB-LRMS: m/z (%, assignment) cluster at 710-716, 710 (67, M⁺). HRMS: Calcd for M⁺ (C₄₀H₄₆N₄O₄Zn): 710.28105, found: 710.27922.

### ZnTBP-=-=(COOH)₂.

### 2-Carboxy-5-(2′-(5′,10′,15′,20′-tetra-n-butylporphyrinato zinc(II))yl)-penta-2,4-dienoic acid

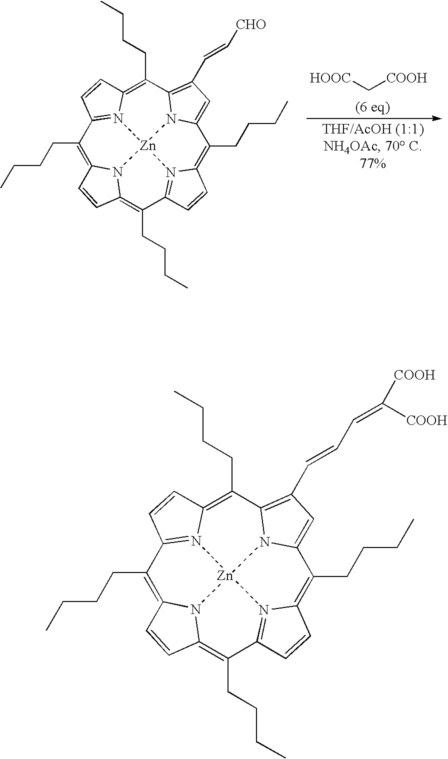

A solution of ZnTBP-=-CHO (150 mg, 230 μmol), malonic acid (144 mg, 1.38 mmol, 6.0 eq) and ammonium acetate (106 mg, 1.38 mmol, 6.0 eq) in a solution of acetic acid:THF (1:1, 14 mL) was heated at 70° C. for 2 hs. On cooling to room temperature H₂O (20 mL) was added, precipitating the crude product. After purification on silica (10% MeOH in DCM) and recrystalisation from THF/acetonitrile ZnTBP-=-=(COOH)₂ (130 mg, 77%) as a dark purple solid was received. ¹H NMR (500 MHz, d₆-DMSO, TMS): δ 11.99 (br s, 1H, COOH), 9.61-9.55 (m, 7H, H_{β-pyrrolic}), 8.59 (d, 1H, J=15.5 Hz, vinyl-H), 8.43 (br s, 1H, COOH), 8.03 (d, 1H, J=15.5 Hz, vinyl-H), 4.97-4.93 (m, 8H, Porph-CH₂-Alk), 2.46-2.40 (m, 8H, Alk-C2), 1.85-1.74 (m, 8H, Alk-C3), 1.23-1.18 (m, 12H, Alk-CH₃). UV-vis (DMF): λₘₐₓ [nm] (log s) 312 (4.31), 434 (5.03), 353 (3.57), 575 (4.01), 621 (3.82). FAB-LRMS: m/z (%, assignment) cluster at 736-742, 736 (100, M⁺). HRMS: Calcd for M⁺ (C₄₂H₄₈N₄O₄Zn): 736.29670, found: 736.29562.

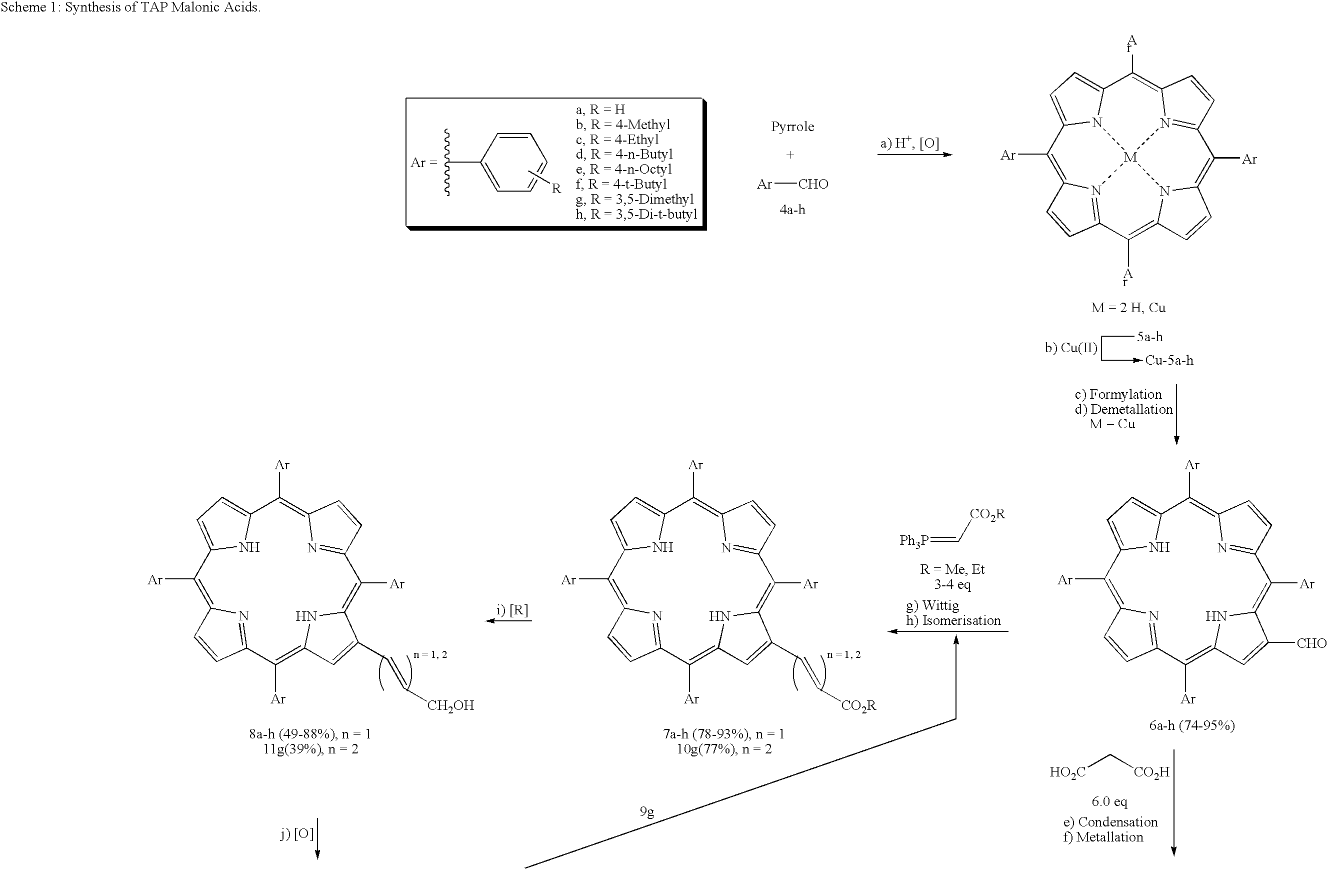

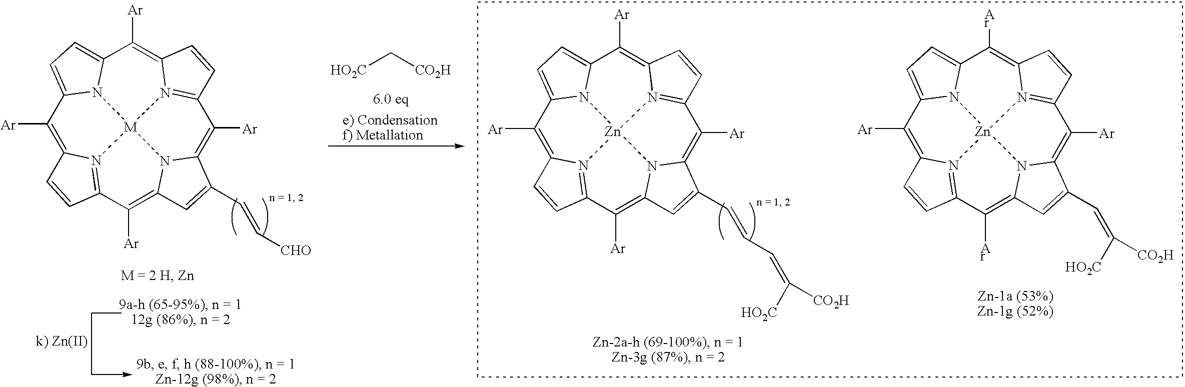

Reagents and conditions: a) i & ii: Adler or Lindsey conditions. b) Cu(OAc)₂.H₂O, CHCl₃:MeOH, reflux (≈30 min). c) POCl₃/DMF, 1,2-DCE, reflux (1.5-7 h). d) Conc. H₂SO₄. e) NH₄OAc (6.0 eq), AcOH:THF, 70° C. (1-7 h), N₂. f) Zn(OAc)₂.2H₂O (2-4 eq), 70° C. (15-60 min), N₂. g) Toluene, reflux (19-42 h), N₂. h) i: I₂, CHCl₃, RT (≈17 h), ii: sat. Na₂S₂O₃ (excess). i) i: DIBAL-H (3 eq), toluene, 0° C. (30-60 min)→RT (30 min), argon; ii: MeOH. j) MnO₂ (14-90 eq), (CH₂Cl₂, CHCl₃, toluene), rt→reflux (0.5-26 h). k) Zn(OAc)₂.2H₂O (1.2 eq), CHCl₃:MeOH, RT (15-30 min).

The required malonic acids Zn-1a, Zn-1g, Zn-2a-h and Zn-3g were synthesised by Knoevenagel condensation of malonic acid from the appropriate aldehydes (where n=0, 1, 2).

The known parent free-base FB porphyri.n compounds 5a-h were first prepared using either Adler-Longo or Lindsey conditions for the condensation of pyrrole with the appropriate benzaldehydes 4a-h. {Kadish, 2000 #1} A new more efficient synthesis of ethylphenyl derivative 5c (20% cf 8%{Berlin, 1998 #12}) was used here, using a 1:1 mixture of refluxing propanoic and octanoic acid. Also a more efficient synthesis of the n-butylphenyl derivative 5d (14% (TFA), 28% (BF₃.OEt₂) cf 2%{Gerasimchuk, 1998 #8}) was achieved here employing Lindsey conditions (additional ¹H NMR characterisation of this compound has been included). The tetra(4-tert-butylpehyl)porphyrin 5f was obtained in 20% using refluxing propionic acid, instead of the μ-wave synthesis reported by Liu et. al . . . {Liu, 2004 #9} The Cu(II) derivatives Cu-5a-h required here for formylation were synthesised from the FB derivatives via the acetate method {Smith, 1975 #19} in near quantitative yields. The Cu(II) derivatives Cu-5c, Cu-5d, Cu-5e are new compounds to the best of our knowledge.

The synthesis of the β-formylporphyrin derivatives 6a-h was achieved using established procedures {Bonfantini, 2002 #6} from their Cu(II) unsubstituted porphyrin derivatives. Formylation and subsequent demetallation of these Cu(II) derivatives yielded the FB aldehydes 6a-h typically in excellent yields (a 95%, b 73%, c 78%, d 85% e 87%, f 74% g 61%, h 94%). Generally formylation times vary from 1.5-7 h depending on a combination of solubility and reactivity of the porphyrin. The tolyl β-formylporphyrin 6b has been previously reported by Buchler et. al. {Buchler, 1988 #7} via the Vilsmeier formylation of the Fe(III)-Cl porphyrin complex, however here the formylation of the Cu(II) derivative using our procedures affords a much more efficient synthesis of 6b (73% cf 50%). Also the 3,5-di-tert-butyl β-formylporphyrin 6h has been previously reported by Aksenova et. al. {Aksenova, 2001 #15} via Vilsmeier formylation and demetallation of Cu-5h.

The synthesis of malonic-acids Zn-1a and Zn-1g where n=0 were obtained by malonic-acid condensation with aldehydes 5a (53%) and 5g (52%), followed by the in situ metallation of the resulting products with zinc acetate.

The extended malonic acids Zn-2a-h where n=2 were synthesised by the Wittig reaction of aldehydes 6a-h with the ethyl or methyl ester of (Triphenyl-λ⁵-phosphanylidene)-acetic acid using the same procedure we have previously reported for Zn-2g.{Schmidt-Mende, 2005 #22} This resulted in isomeric cis/trans mixtures of the vinyl esters 7a-h. Isomerization of these mixtures to the all-trans esters 7a-h was achieved efficiently with iodine. DIBAL-H reduction of esters 7a-h, followed by oxidation of the alcohols 8a-h, with MnO₂ afforded in excellent yields the allylaldehydes 9a-h. Near quantitative yields of the extended malonic-acids Zn-2a-h was subsequently obtained by malonic-acid condensation with these aldehydes, some aldehydes were metallated prior to before condensation. However, it was found necessary to add extra zinc(II) acetate after condensation, as often partial demetallation occurs during the reaction.

The extended-extended (i.e. n=3) malonic acid Zn-3g was synthesised by simply resubjecting the extended aldehyde 9g through the previous synthesis protocol for the extended malonic acids Zn-2a-h. This gave the extended-extended ester 10g (77%), alcohol 11g (39%), aldehydes 12g (86%) and Zn-12g (98%). Malonic acid condensation with Zn-12g resulted in an 87% yield of the desired product Zn-3g.

The analytical and spectroscopic data for these compounds are fully consistent with their structures. All compounds were characterised by ¹H NMR spectroscopy, UV-visible spectroscopy and FAB HRMS. ¹H NMR spectra were typical of β-substituted tetraphenylporphyrins with no unusual features. Where necessary, ¹H NMR assignments were aided by 2D NMR (COSY & HETCOR) spectra. The HRMS data was as expected for the required compounds.

### Acids

### Zn-1a, ZnTPP-=(COOH)₂

### 2-Carboxy-3-(2′-(5′,10′,15′,20′-tetraphenylporphyrinato zinc(II))yl)-prop-2-enoic acid

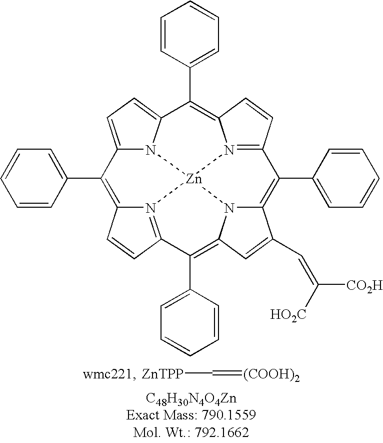

A solution of 6a{Bonfantini, 2002 #6} (250 mg, 389 μmol), malonic acid (242 mg, 2.33 mmol, 6.0 eq) and ammonium acetate (180 mg, 2.33 mmol, 6.0 eq) in a solution of acetic acid (3.0 mL) and CHCl₃ (6.0 mL) was heated at 70° C. for 20 h. On cooling to RT the solution was filtered and a solid precipitated with acetonitrile. The solid was dissolved in THF with excess Et₃N and filtered, then acidified with acetic acid (3.0 mL). The THF was then removed by azeotrope distillation using acetonitrile in vacuo. Zn(OAc)₂.2H₂O (171 mg, 778 μmol, 2.0 eq) was added to the resulting red solution with stirring at RT. After 1 h the solution was filtered through a silica gel plug with acetonitrile. The solvent was reduced in vacuo and the resulting purple precipitate collected by filtration (#4) to give the Zn-1a (163 mg, 53%). ¹H NMR (400 MHz, d₆-DMSO, TMS): δ 7.46 (br s, 1H, H₃), 7.71-7.83 (m, 12H, H_{m.p-Ph}), 8.04-8.06 (m, 2H, H_{o-Ph}), 8.14-8.20 (m, 6H, H_{o-Ph}), 8.66 and 8.74 (ABq, 2H, ³J=4.7, 4.7 Hz, H_{β-pyrrolic}) 8.76 (s, 2H, H_{β-pyrrolic}), 8.77 (s, 2H, H_{β-pyrrolic}), 8.94 (br s, 1H, H_{3′(β-pyrrolic)}), 12.8 (br s, 2H, CO₂H). UV-vis (THF): λₘₐₓ [nm] (ε×10⁻³) 319 (17.1), 431 (271), sh 525 (3.20), 562 (16.6), 603 (5.67). FAB-LRMS: m/z (%, assignment) cluster at 790-796, 790 (100, M⁺). HRMS: Calcd for M⁺ (C₄₈H₃₀N₄O₄Zn): 790.1559, found: 790.1546.

### Zn-1g, ZnTXP-=(COOH)₂

### 2-Carboxy-3-(2′-(5′10′,15′,20′-tetra(3″,5″-dimethylphenyl)porphyrinato zinc(II))yl)-prop-2-enoic acid

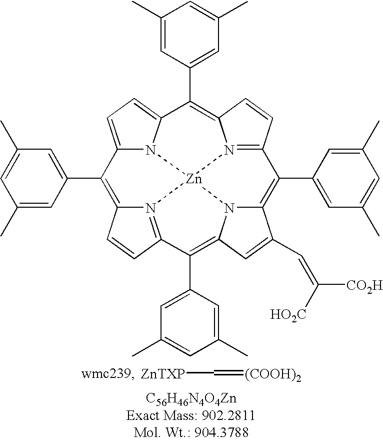

A solution of 6g{Bonfantini, 2002 #6} (250 mg, 331 μmol), malonic acid (207 mg, 1.99 mmol, 6.0 eq) and ammonium acetate (153 mg, 1.99 mmol, 6.0 eq) in a solution of acetic acid (3.0 mL) and CHCl₃ (3.0 mL) was heated at 70° C. for 2 h. On cooling to RT acetonitrile (≈50 mL) was added and the solvent removes in vacuo. CHCl₃ (100 mL) was added and the solution filtered (#4). Zn(OAc)₂.2H₂O (145 mg, 661 μmol, 2.0 eq) in MeOH (5.0 mL) was added to the resulting solution and heated at 70° C. for 15 min. The CHCl₃ was then removed by azeotrope distillation using MeOH in vacuo. Sufficient H₂O was added, precipitating the crude product (235 mg) as a purple solid. The crude product was column chromatographed (silica, 37 mm_{dia}×100 mm, acetonitrile:CHCl₃ (9:1)→acetonitrile:CHCl₃:MeOH (8:1:1)) collecting the major green/red band in two fractions. Additional acetonitrile was added and the volume reduced by half in vacuo. The products were then precipitated from solution with H₂O and collected by filtration (#4) to give the Zn-1g (155 mg, 52%) as a purple powder. ¹H NMR (400 MHz, d₆-DMSO): δ 2.48-2.60 (m, 24H, H_{Me-Xyl}), 7.34 (s, 1H, H_{p-Ar}), 7.42 (br s, 4H, 3H_{p-Ar}+1H₃), 7.66 (s, 2H, H_{o-Ar}), 7.76 (s, 2H, H_{o-Ar}), 7.78 (s, 4H, H_{o-Ar}), 8.75-8.80 (m, 6H, H_{β-pyrrolic}) 8.92 (br s, 1H, H_{3′(β-pyrrolic)}), 12.8 (br s, 2H, CO₂H). Assignments aided by COSY & LR-COSY spectra. UV-vis (THF): λₘₐₓ [nm] (ε×10⁻³) 318 (19.3), 432 (314), sh 526 (3.32), 563 (18.3), 604 (6.81). FAB-LRMS: m/z (%, assignment) cluster at 901-907, 902 (100, M⁺). HRMS: Calcd for M⁺ (C₅₆H₄₆N₄O₄Zn): 902.2811, found: 902.2798.

### Zn-2a, ZnTPP-=-=(COOM)₂

### 2-Carboxy-5-(2′-(5′,10′,15′,20′-tetraphenylporphyrinato zinc(II))yl)-penta-2,4-dienoic acid

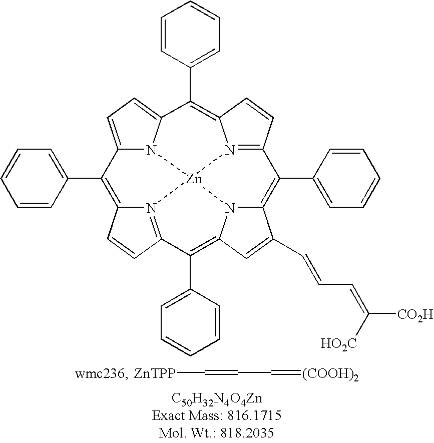

A solution of 9a (175 mg, 262 μmol), malonic acid (164 mg, 1.58 mmol, 6.0 eq) and ammonium acetate (121 mg, 1.57 mmol, 6.0 eq) in a solution of acetic acid (5.0 mL) and CHCl₃ (3.0 mL) was heated at 70° C. for 3 h. On cooling to room temperature, MeOH (25 mL) was added and the resulting precipitate collected (#4). The solid was dissolved CHCl₃ (100 mL) and Zn(OAc)₂.2H₂O (230 mg, 1.05 mmol, 4.0 eq) in MeOH (15 mL) was added to the resulting solution heated at 70° C. for 15 min. On cooling to room temperature the CHCl₃ was then removed by azeotrope distillation using MeOH in vacuo. Sufficient H₂O was added, precipitating the product to give Zn-2a (186 mg, 87%) as a purple solid. ¹H NMR (Broadened Spectrum, 400 MHz, d₆-DMSO, TMS): δ 6.41 (d, 1H, ³J=14.7 Hz, H_{5 (pentadienyl)}), 7.78 (br s, 1H, H_{3 (pentadienyl)}), 7.73-7.92 (m, 13H, 12H_{m.p-Ar}+1H (pentadienyl)), 8.07-8.16 (m, 8H, 6H_{o-Ar}), 8.66-8.81 (m, 6H, H_{β-pyrrolic}), 8.89 (br s, 1H, H_{3′(β-pyrrolic)}). UV-vis (THF): λₘₐₓ [nm] (ε×10⁻³) 320 (29.5), 440 (172), 569 (19.9), 612 (13.6). FAB-LRMS: m/z (%, assignment) cluster at 816-821, 816 (100, M⁺). HRMS: Calcd for M⁺ (C₅₀H₃₂N₄O₄Zn): 816.1715, found: 816.1716.

### Zn-2b, ZnTTP-=-=(COOM)₂

### 2-Carboxy-5-(2′-(5′,10′,5′,20′-tetra(4″-methylphenyl)porphyrinato zinc(II))yl)-penta-2,4-dienoic acid

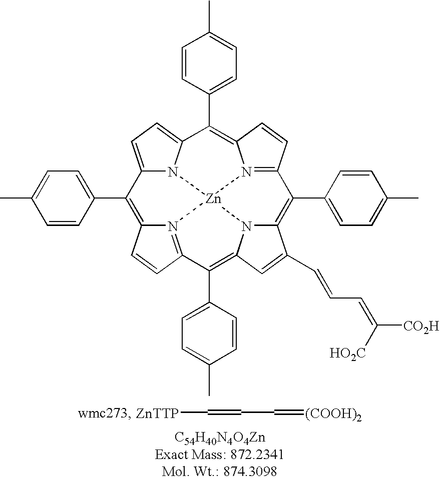

A solution of Zn-9b (110 mg, 0.140 mmol), malonic acid (87 mg, 0.84 mmol, 6.0 eq) and ammonium acetate (65 mg, 0.84 mmol, 6.0 eq) in a solution of AcOH:THF (1:1, 5.0 mL) was heated at 70° C. for 90 min. Zn(OAc)₂.2H₂O (123 mg, 0.56 mmol, 4.0 eq) was added and the solution heated at 70° C. for 15 min. On cooling to room temperature sufficient H₂O was added, precipitating the crude product, R_{f}=0.13 (silica, 5% MeOH: 1% AcOH:CH₂Cl₂). Recrystalisation from CHCl₃:hexane gave Zn-2b (114 mg, 94%) as a dark purple powder. ¹H NMR (400 MHz, d₆-DMSO, TMS): δ 2.65 (s, 6H, H_{Me-Ar}), 2.68 (s, 3H, H_{Me-Ar}), 2.70 (s, 3H, H_{Me-Ar}), 6.45 (d, 1H, ³J=14.9 Hz, H_{5 (pentadienyl)}), 6.99 (d, 1H, ³J=11.6 Hz, H_{3. (pentadienyl)}), 7.38 (appt t, 1H, ³J=12.8 Hz, H_{4 (pentadienyl)}), 7.56-7.61 (m, 8H, H_{m-Ar}), 7.94 (d, 2H, ³J=7.7 Hz, H_{o-Ar}), 8.01-8.07 (m, 6H, H_{o-Ar}), 8.70-8.80 (m, 6H, H_{β-pyrrolic}), 8.94 (br s, 1H, H_{3′(β-pyrrolic)}), Assignments aided by COSY spectra. UV-vis (THF): λₘₐₓ [nm] (ε×10⁻³) 329 (30.0), 446 (142), 570 (19.3), 621 (16.3). FAB-LRMS: m/z (%, assignment) cluster at 872-877, 872 (100, M⁺). HRMS: Calcd for M⁺ (C₅₄H₄₀N₄O₄Zn): 872.2341, found: 872.2343.

### Zn-2c, ZnTEPP-=-=(COOH)₂

### 2-Carboxy-5-(2′-(5′,10′,15′,20′-tetra(4″-ethylphenyl)porphyrinato zinc(II))yl)-penta-2,4-dienoic acid

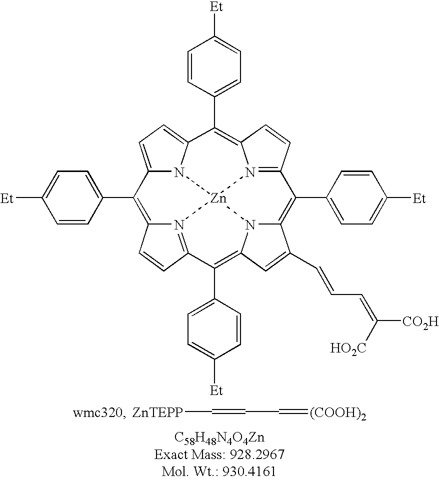

A solution of 9c (200 mg, 0.256 mmol), malonic acid (160 mg, 1.54 mmol, 6.0 eq) and ammonium acetate (118 mg, 1.53 mmol, 6.0 eq) in a solution of AcOH:THF (1:1, 10.0 mL) was heated at 70° C. for 135 min under N₂ atmosphere. Zn(OAc)₂.2H₂O (225 mg, 1.03 mmol, 4.0 eq) was added and the solution heated at 70° C. for 30 min. On cooling to room temperature the mixture was filtered. To the filtrate was added CHCl₃ (100 mL). The organic layer was then washed with H₂O (100 mL×2) buffering the second wash with 2 M aqueous H₃PO₄ (0.5 mL, pH 2.5). The organic layer was carefully separated and the solvent removed in vacuo. Recrystalisation from CHCl₃:hexane gave Zn-2c (225 mg, 95%) as a dark purple powder. ¹H NMR (500 MHz, d₆-DMSO, TMS): δ 1.44-1.55 (m, 12H, CH₃), 2.92-3.01 (m, 8H, CH₂), 6.47 (d, 1H, ³J=15.1 Hz, H_{5 (pentadienyl)}), 6.97 (d, 1H¹³J=11.6 Hz, H_{3. (pentadienyl)}), 7.34 (appt t, 1H, ³J=12.9 Hz, H_{4 (pentadienyl)}), 7.58-7.64 (m, 8H, H_{m-Ar}), 7.96 (d, 2H, ³J=7.9 Hz, H_{o-Ar}), 8.05 (d, 4H, ³J=7.8 Hz, H_{o-Ar}), 8.09 (d, 2H, ³J=7.9 Hz, H_{o-Ar}), 8.71-8.77 (m, 6H, H_{β-pyrrolic}) 8.90 (s, 1H, H_{3′(β-pyrrolic)}), 13.3 (br s, COOH). Assignments aided by COSY spectra. UV-vis (DMF): λₘₐₓ [nm] (ε×10⁻³) 317 (27.6), 361 (19.9), 444 (187), 538 (5.72), 572 (19.9), 612 (11.1). FAB-LRMS: m/z (%, assignment) cluster at 927-933, 928 (100, M⁺). HRMS: Calcd for M⁺ (C₅₈H₄₈N₄O₄Zn): 928.2967, found: 928.2966.

### Zn-2d, ZnTnBPP-=-=(COOH)₂

### 2-Carboxy-5-(2′-(5′,10′,15′,20′-tetra(4″-buylphenyl)porphyrinato zinc(II))yl)-penta-2,4-dienoic acid

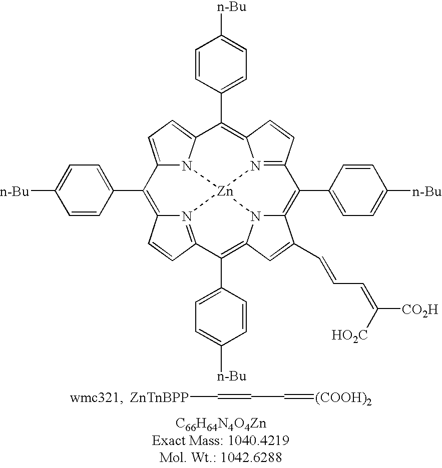

A solution of 9d (190 mg, 0.213 mmol), malonic acid (133 mg, 1.28 mmol, 6.0 eq) and ammonium acetate (98 mg, 1.27 mmol, 6.0 eq) in a solution of AcOH:THF (1:1, 9.0 mL) was heated at 70° C. for 135 min under N₂ atmosphere. Zn(OAc)₂.2H₂O (187 mg, 0.852 mmol, 4.0 eq) was added and the solution heated at 70° C. for 30 min. On cooling to room temperature the mixture was filtered. To the filtrate was added CHCl₃ (100 mL). The organic layer was then washed with H₂O (≈100 mL×2) buffering the second wash with 2 M aqueous H₃PO₄ (0.5 mL, pH z 2.5). The organic layer was carefully separated and the solvent removed in vacuo. Recrystalisation from CHCl₃:hexane gave Zn-2d (214 mg, 96%) as a dark purple powder. (Often a little THF may be needed, to be added, to the CHCl₃ to aid redissolving). ¹H NMR (500 MHz, d₆-DMSO, TMS): δ 1.05-1.09 (m, 12H, CH₂CH₂CH₂CH₃), 1.48-1.61 (m, 8H, CH₂CH₂CH₂CH₃), 1.80-1.93 (m, 8H, CH₂CH₂CH₂CH₃), 2.87-2.96 (m, 8H, CH₂CH₂CH₂CH₃), 6.52 (d, 1H, ³J=15.0 Hz, H_{5 (pentadienyl)}), 6.96 (d, 1H, ³J=11.7 Hz, H_{3. (pentadienyl)}), 7.32 (br m, 1H, H_{4 (pentadienyl)}), 7.53-7.60 (m, 8H, H_{m-Ar}), 7.94 (d, 2H, ³J=7.9 Hz, H_{o-Ar}), 8.01 (d, 4H, ³J=7.9 Hz, H_{o-Ar}), 8.07 (d, 2H, ³J=8.0 Hz, H_{o-Ar}), 8.69-8.73 (m, 6H, H_{β-pyrrolic}), 8.89 (s, 1H, H_{3′(β-pyrrolic)}), Assignments aided by COSY spectra. UV-vis (DMF): λₘₐₓ [nm] (ε×10⁻³) 317 (29.4), 363 (21.1), 444 (204), sh 533 (5.67), 572 (21.3), 612 (11.8). FAB-LRMS: m/z (%, assignment) cluster at 1039-1047, 1040 (100, M⁺). HRMS: Calcd for M⁺ (C₆₆H₆₄N₄O₄Zn): 1040.4219, found: 1040.4192.

### Zn-2e, ZnTOPP-=-=(COOH)₂

### 2-Carboxy-5-(2′-(5′,10′,15′,20′-tetra(4″-octylphenyl)porphyrinato zinc(II))yl)-penta-2,4-dienoic acid

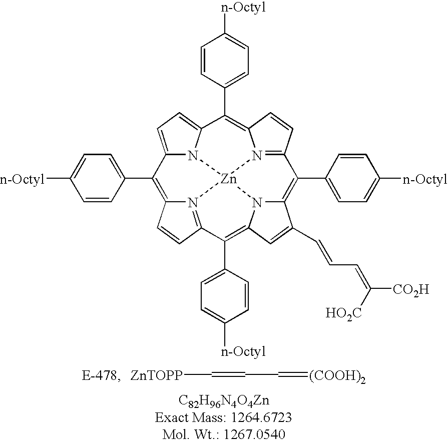

A solution of Zn-9e (236 mg, 200 μmol), malonic acid (125 mg, 1.20 mmol, 6.0 eq) and ammonium acetate (92 mg, 1.20 mmol, 6.0 eq) in a solution of AcOH:THF (1:1, 12 mL) was heated at 70° C. for 90 min. On cooling to room temperature H₂O (50 mL) was added, precipitating the crude product. After purification on silica (1% AcOH, 5% MeOH in DCM) and recrystalisation from DCM/acetonitrile Zn-2e (175 mg, 69%) as a dark purple powder was received. ¹H NMR (500 MHz, d₆-DMSO, TMS): δ 0.91 (br s, 12H, Alk-CH₃). 1.59-1.28 (m, 40H, Alk), 1.95-1.81 (m, 8H, Alk), 2.98-2.87, m, 8H, Ar—CH₂-Alk), 6.51 (d, 1H, ³J=15.5 Hz, H_{ethenyl}), 6.97 (d, 1H, ³J=15.5 Hz, H_{ethenyl}), 7.35 (br s, 1H, H_{ethenyl}), 7.63-7.51 (m, 8H, H_{Ar}), 8.13-7.90 (m, 8H, H_{o-Ar}), 8.78-8.67 (m, 6H, H_{β-pyrrolic}), 8.89 (s, 1H, H_{3′(β-pyrrolic)}), 13.35 (br s, 1H, COOH). UV-vis (DMF): λₘₐₓ [nm] (ε×10⁻³) 317 (33.8), 444 (186), 534 (7.65), 572 (20.8), 612 (12.2). FAB-LRMS: m/z (%, assignment) cluster at 1262-1268, 1265 (94, MH⁺). HRMS: Calcd for MH⁺ (C₈₂H₉₇N₄O₄Zn): 1265.6801, found: 1265.6827.

### Zn-2f, ZnT4BP-=-=(COOM)₂

### 2-Carboxy-5-(2′-(5′,10′,15′,20′-tetra(4″-tert-butylphenyl)porphyrinato zinc(II))yl)-penta-2,4-dienoic acid

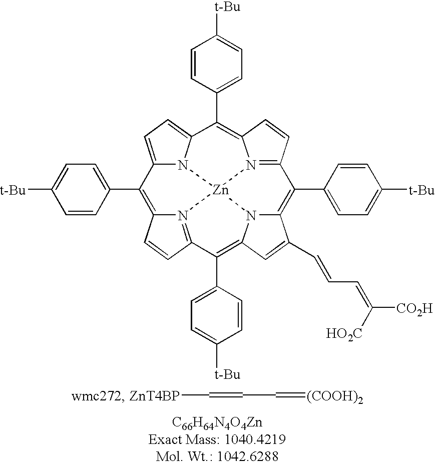

A solution of Zn-9f (110 mg, 115 μmol), malonic acid (72 mg, 0.69 mmol, 6.0 eq) and ammonium acetate (53 mg, 0.69 mmol, 6.0 eq) in a solution of AcOH:THF (1:1, 5.0 mL) was heated at 70° C. for 100 min. Zn(OAc)₂.2H₂O (101 mg, 0.46 mmol, 4.0 eq) was added and the solution heated at 70° C. for 15 min. On cooling to room temperature sufficient H₂O was added, precipitating the crude product, R_{f}=0.33 (silica, 5% MeOH: 1% AcOH:CH₂Cl₂). Recrystalisation from THF:H₂O gave Zn-2f (116 mg, 96%) as a black solid. ¹H NMR (400 MHz, d₈-THF, TMS): δ 1.6 (s, 18H, C(CH₃)₃), 1.63 (s, 9H, C(CH₃)₃), 1.65 (s, 9H, C(CH₃)₃), 6.80 (d, 1H, ³J=15.0 Hz, H_{5 (pentadienyl)}), 7.53 (d, 1H, ³J=11.8 Hz, H_{3 (pentadienyl)}), 7.74-7.83 (m, 8H, H_{m-Ar}), 8.03 (d, 2H, ³J=8.1 Hz, H_{o-Ar}), 8.10-8.15 (m, 6H, H_{o-Ar}), 8.25 (dd, 1H, ³J=14.9, 11.7 Hz, H_{4 (pentadienyl)}), 8.78-8.83 (m, 6H, H_{β-pyrrolic}), 9.19 (s, 1H, H_{3′(β-pyrrolic)}), Assignments aided by COSY spectra. UV-vis (THF): λₘₐₓ [nm] (ε×10⁻³) 330 (25.4), 448 (112), 572 (15.8), 624 (14.6). FAB-LRMS: m/z (%, assignment) cluster at 1040-1046, 1040 (100, M⁺). HRMS: Calcd for M⁺ (C₆₆H₆₄N₄O₄Zn): 1040.4219, found: 1040.4212.

### Zn-2g, ZnTXP-=-=(COOH)₂

### 2-Carboxy-5-(2′-(5′,10′,15′,20′-tetra(3″,5″-dimethylphenyl)porphyrinato zinc(II))yl)-penta-2,4-dienoic acid

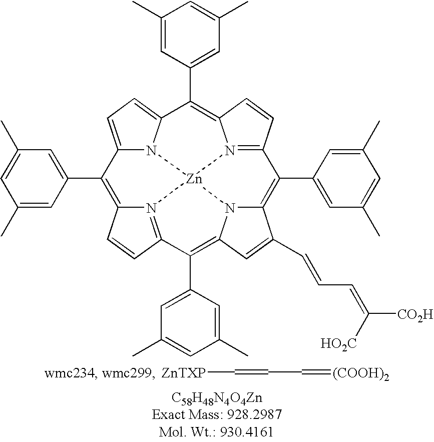

A solution of 9g (200 mg, 256 μmol), malonic acid (160 mg, 1.54 mmol, 6.0 eq) and ammonium acetate (118 mg, 1.53 mmol, 6.0 eq) in a solution of AcOH (5.0 mL) was heated at 70° C. for 3 h. Zn(OAc)₂.2H₂O (222 mg, 1.01 mmol, 4.0 eq) was added to the resulting red solution heated at 70° C. for 15 min. On cooling to room temperature sufficient H₂O was added, precipitating the product to give Zn-2g (238 mg, 100%) as a purple solid. ¹H NMR (400 MHz, d₆-DMSO, TMS): δ 2.54 (s, 6H, H_{Me-Xyl}), 2.58 (s, 12H, H_{Me-Xyl}), 2.60 (s, 6H, H_{Me-Xyl}), 6.51 (d, 1H¹³J=15.1 Hz, H_{5 (pentadienyl)}), 7.16 (d, 1H, ³J=11.6 Hz, H_{3 (pentadienyl)}), 7.42 (s, 2H, H_{p-Ar}), 7.459 (s, 1H, H_{p-Ar}), 7.51 (s, 1H, H_{p-Ar}), 7.691 (s, 2H, H_{o-Ar}), 7.77-7.81 (m, 7H, 6H_{o-Ar}+1H_{4 (pentadienyl)}), 8.74-8.80 (m, 6H, H_{β-pyrrolic}), 8.91 (br s, 1H, H_{3′(β-pyrrolic)}), Assignments aided by COSY & LR-COSY spectra. UV-vis (THF): λₘₐₓ [nm] (ε×10⁻³) 326 (24.0), sh 431 (116), 443 (120), 570 (15.9), 618 (12.5). FAB-LRMS: m/z (%, assignment) cluster at 928-934, 928 (100, MH⁺). HRMS: Calcd for M⁺ (C₅₈H₄₈N₄O₄Zn): 928.2967, found: 928.2966.

### Zn-2h, ZnTBP-=-=(COOH)₂

### 2-Carboxy-5-(2′-(5′,10′,15′,20′-tetra(3″,5″-di-tert-butylphenyl)porphyrinato zinc(II))yl)-penta-2,4-dienoic acid

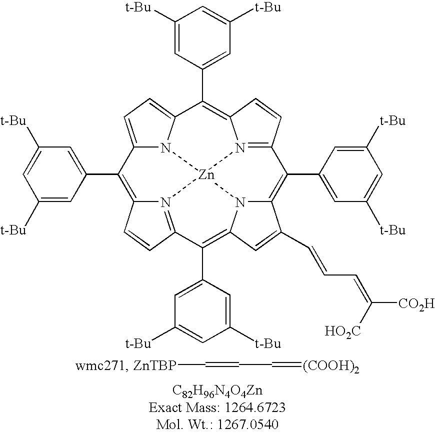

A solution of Zn-9h (100 mg, 84.7 μmol), malonic acid (53 mg, 0.51 mmol, 6.0 eq) and ammonium acetate (39 mg, 0.51 mmol, 6.0 eq) in a solution of AcOH:THF (1:1, 5.0 mL) was heated at 70° C. for 3 h. Zn(OAc)₂.2H₂O (74 mg, 0.34 mmol, 4.0 eq) was added and the solution heated at 70° C. for 15 nm in. On cooling to room temperature sufficient H₂O was added, precipitating the crude product. Recrystalisation from THF:H₂O gave Zn-2h (99.2 mg, 92%) as a dark green powder. ¹H NMR (400 MHz, d₈-THF, TMS): δ 1.50 (s, 18H, C(CH₃)₃), 1.53 (s, 36H, C(CH₃)₃), 1.56 (s, 18H, C(CH₃)₃), 6.63 (d, 1H, ³J=15.0 Hz, H_{5 (pentadienyl)}), 7.60 (d, 1H, ³J=11.7 Hz, H_{3 (pentadienyl)}), 7.85-7.88 (m, 3H, H_{P-Ar}), 7.99-8.02 (m, 3H, 1H_{P-Ar}+2H_{o-Xyl}), 8.07 (d, 4H, ⁴J=1.8 Hz, H_{o-Ar}), 8.12 (d, 2H, ⁴J=1.8 Hz, H_{o-Ar}), 8.21-8.31 (m, 1H, H_{4 (pentadienyl)}), 8.78-8.86 (m, 6H, H_{β-pyrrolic}), 9.27 (s, 1H, H_{3′(β-pyrrolic)}), Assignments aided by COSY spectra UV-vis (THF): λₘₐₓ [nm] (ε×10⁻³) 330 (26.1), sh 416 (73.4), 449 (116), 571 (16.5), 624 (16.5). FAB-LRMS: m/z (%, assignment) cluster at 1264-1271, 1265 (95, M⁺). HRMS: Calcd for M⁺ (C₈₂H₉₆N₄O₄Zn): 1264.6723, found: 1264.6692.

### Zn-3g, ZnTXP-=-=-=(COOH)₂

### 2-Carboxy-5-(2′-(5′,10′,15′,20′-tetra(3″,5″-dimethylphenyl)porphyrinato zinc(II))yl)-hepta-2,4,6-trienoic acid

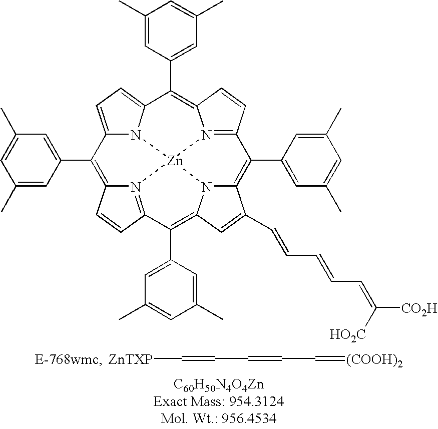

A solution of Zn-12g (184 mg, 210 μmol), malonic acid (132 mg, 1.20 mmol, 6.0 eq) and ammonium acetate (92 mg, 1.20 mmol, 6.0 eq) in a solution of AcOH:THF (1:1, 10 mL) was heated at 70° C. for 90 min. On cooling to room temperature H₂O (50 mL) was added, precipitating the crude product. The solid was column chromatographed (silica, AcOH:MeOH:DCM (1:5:94)) to give Zn-3g (174 mg, 87%) as a dark purple powder. ¹H NMR (500 MHz, d₆-DMSO, TMS): 2.60-2.56 (m, 24H, H_{Me-Xyl}), 6.32 (d, 1H, ³J=14.6 Hz, H_{ethenyl}), 6.57 (dd, 1H, ³J=11.0, 14.6 Hz, H_{ethenyl}), 7.01 (br s, 1H, H_{ethenyl}), 7.24 (dd, 1H, ³J=10.6, 15.1 Hz, H_{ethenyl}), 7.41 (d, 1H, ³J=10.6 Hz, H_{ethenyl}), 7.79-7.43 (m, 12H, H_{Ar}), 8.79-8.82 (m, 6H, H_{β-pyrrolic}), 8.96 (s, 1H, H_{3′(β-pyrrolic)}), 13.28 (br s, 1H, COOH). UV-vis (DMF): λₘₐₓ [nm] (ε×10⁻³) 343 (22.4), 442 (106), 572 (14.9), 612 (10.0). FAB-LRMS: m/z (%, assignment) cluster at 952-960, 954 (100, M⁺). HRMS: Calcd for M⁺ (C₆₀H₅₀N₄O₄Zn): 954.3124, found: 954.3140.

### Methoxyphenyl Malonic Acids

### CuTMPP-=(COOH)₂

### 2-Carboxy-3-(2′-(5′,10′,15′, 20′-tetra(4″-methoxyphenyl)porphyrinato copper(II))yl)-prop-2-enoic acid

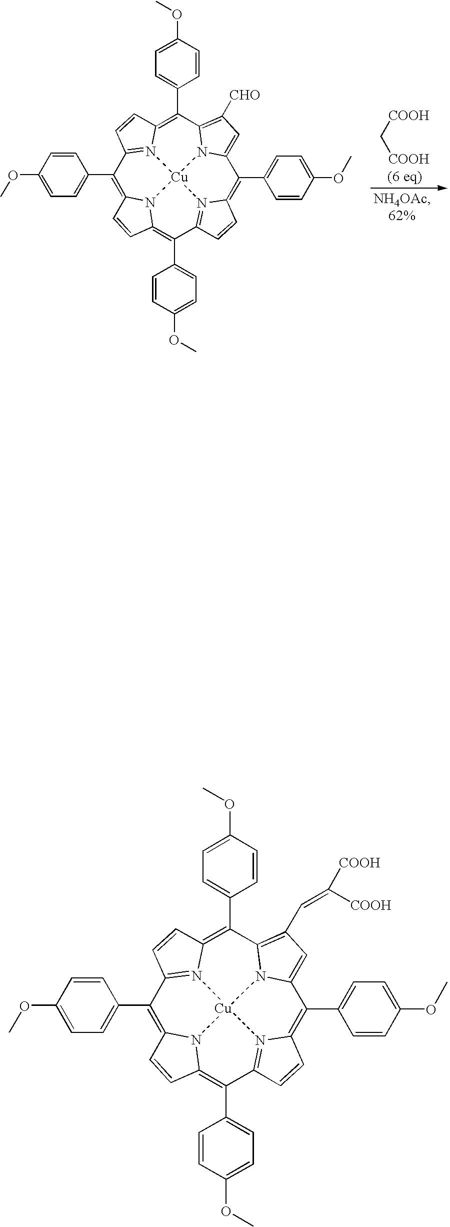

A solution of CuTMPP-CHO (165 mg, 200 μmol), malonic acid (124 mg, 1.20 mmol, 6.0 eq) and ammonium acetate (93 mg, 1.20 mmol, 6.0 eq) in a solution of acetic acid:THF (1:2, 15 mL) was heated at 70° C. for 180 min. On cooling to room temperature acetonitrile (50 mL) was added, precipitating the product. CuTMPP-=(COOH)₂ (100 mg, 62%) as a dark purple powder was received.

### H₂TMPP-=(COOM)₂

### 2-Carboxy-3-(5′,10′,15′,20′-tetra(4″-methoxyphenyl)porphyrin-2′-yl)-prop-2-enoic acid

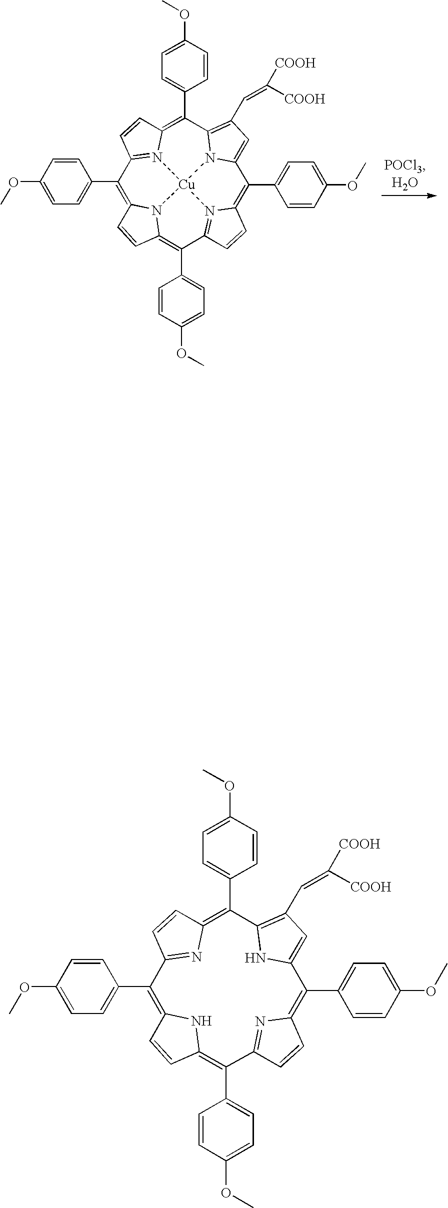

CuTMPP-=(COOH)₂ (201 mg, 220 μmol) was dissolved in POCl₃ (20 ccm) and cooled to 0° C. then water (2 mL) was added. The resulting mixture was stirred at 0° C. for 30 min then poured into ice (100 g) and neutralized by ammonia. The dark solid was filtered off. After recryst. from THF/acetonitrile mixture H₂TMPP-=(COOH)₂ (102 mg, 55%) was received. ZnTMPP-=(COOH)₂

### 2-Carboxy-3-(2′-(5′, 10′,15′,20′-tetra(4″-methoxyphenyl)porphyrinato zinc(II))yl)-prop-2-enoic acid

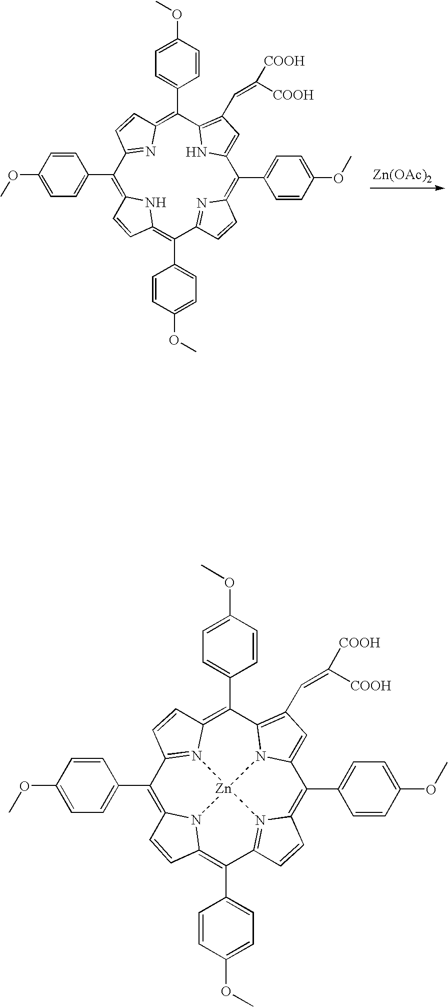

H₂TMPP-=(COOH)₂ (85 mg, 100 μmol) was dissolved in DCM (50 ccm) and zinc acetate dehydrate (33 mg 0.15 mmol) was added in MeOH (10 mL). The resulting mixture was stirred for 0.5 h. The solvents were removed under vacuo and recryst. from THF/acetonitrile mixture to give ZnTMPP-=(COOH)₂ (30 mg, 77%).

### CuTMPP-=-CO₂Et.

Copper 3-trans-(5′,10′,15′,20′-tetra(4″-methoxphenyl)porphyrin-2′-yl)-acrylic acid methyl ester.

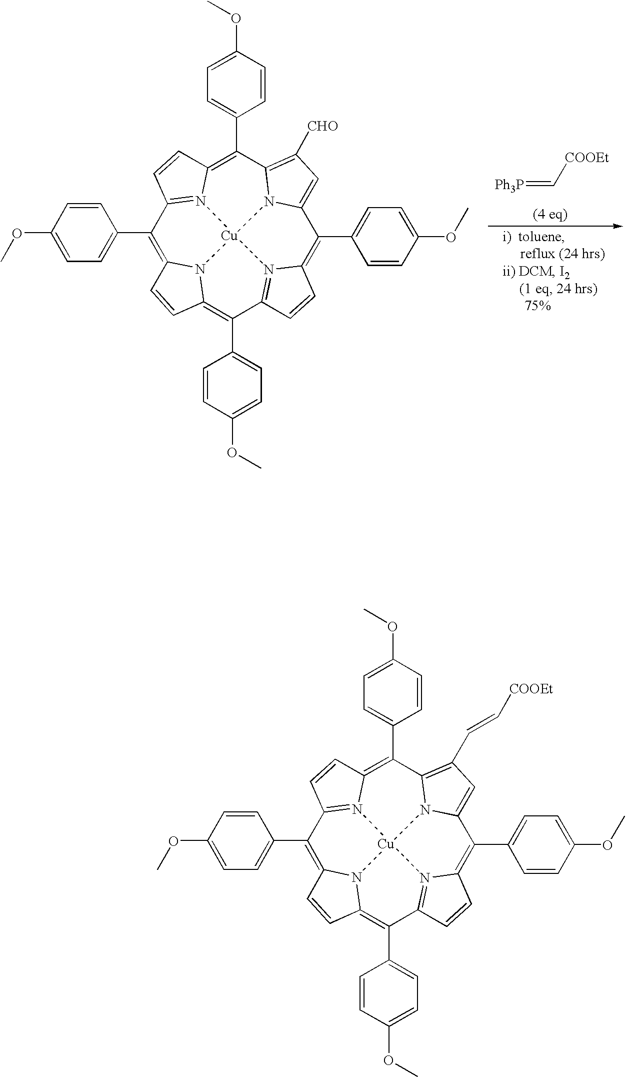

### Wittig:

A solution of CuTMPP-CHO (0.412 g, 0.50 mmol) and phosphorane Ph₃P═CHCO₂Et (0.649 g, 2.00 mmol, 4 eq) in dry toluene (50 mL) was heated at reflux temperature under Argon. After 24 hs, TLC analysis (silica, toluene) indicated that all of the starting material CuTMPP-CHO had been consumed. After cooling to RT the solvent was removed in vacuo. The residue was column chromatographed (silica, 2% of AcOEt in toluene) collecting the major purple colored fraction to give a cis/trans isomeric mixture of CuTBPP-=-CO₂Et (0.382 g,) as a reddish solid.

### Isomerisation:

The isomeric mixture was dissolved in DCM (35 mL) and 12 (108 mg, 0.43 mmol, 1.0 eq) was added. After stirring at RT for 24 hs in darkness, excess sat. Na₂S₂O₃ (≈40 mL) was added and stirring continued for 30 min. The organic layer was separated, dried (MgSO₄) and the product precipitated with methanol to give trans CuTMPP-=-CO₂Et (0.336 g, 75% overall) as a purple powder. FAB-LRMS: m/z (%, assignment) cluster at 889-902, 893 (100, M⁺). HRMS: Calcd for M⁺ (C₅₃H₄₂N₄O₆Cu): 893.24003, found: 893.24021.

### CuTMPP-=-CH₂OH

### Copper 3-(5′,10′,15′,20′-tetra(4″-methoxyphenyl)porphyrin-2′-yl)-allylhydroxide

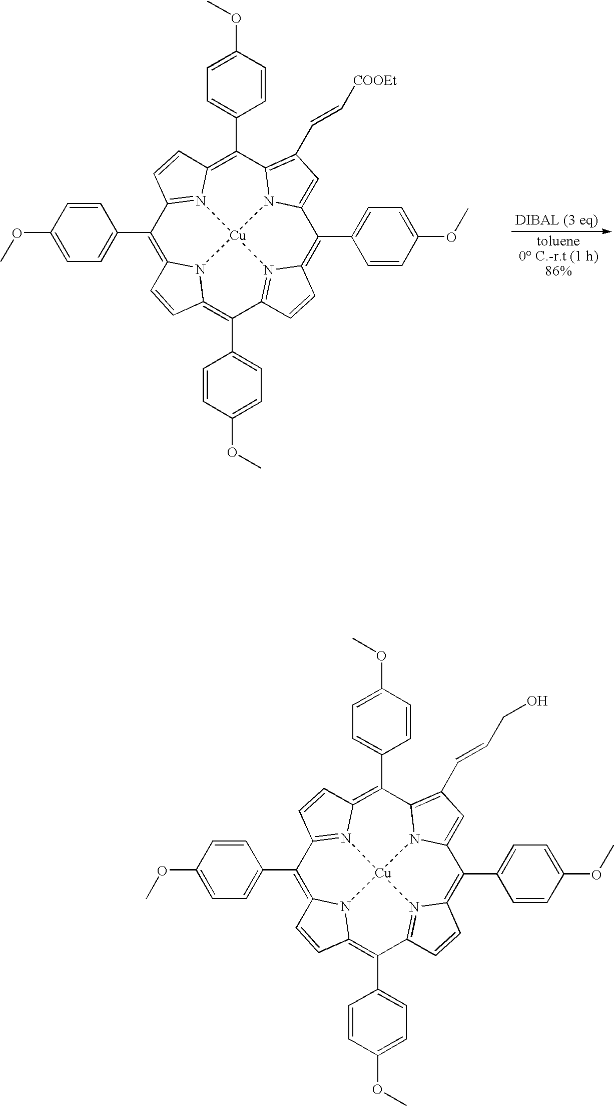

DIBAL-H (1.8 mL, 1.5 M in toluene, 2.70 mmol, 3.0 eq) was added to a solution of TOPP-=-CO₂Et (0.805 g, 0.90 mmol) in dry toluene (50 mL) under argon atmosphere at 0° C. After 30 min the reaction was allowed to warm to RT. After another 30 min MeOH (5.0 mL) added followed by 10% NaOH (50 mL). CH₂Cl₂ (150 mL) was added and the organic layer separated, dried (MgSO₄) and the solvent removed in vacuo. The residue was column chromatographed (silica, 2% of EtO2 in toluene) collecting the first major red colored fraction. Recrystallization from CH₂Cl₂:MeOH gave CuTMPP-=-CH₂OH (660 mg, 86%) as a purple solid. FAB-LRMS: m/z (%, assignment) cluster at 847-866, 851 (100, M⁺).

HRMS: Calcd for M⁺ (C₅₁H₄₀N₄O₅Cu): 851.22947, found: 851.22693.

### CuTMPP-=-CHO

### Copper 3-(5′, 10′, 15′,20′-tetra(4″-methoxyphenyl)porphyrin-2′-yl)-allylaldehyde

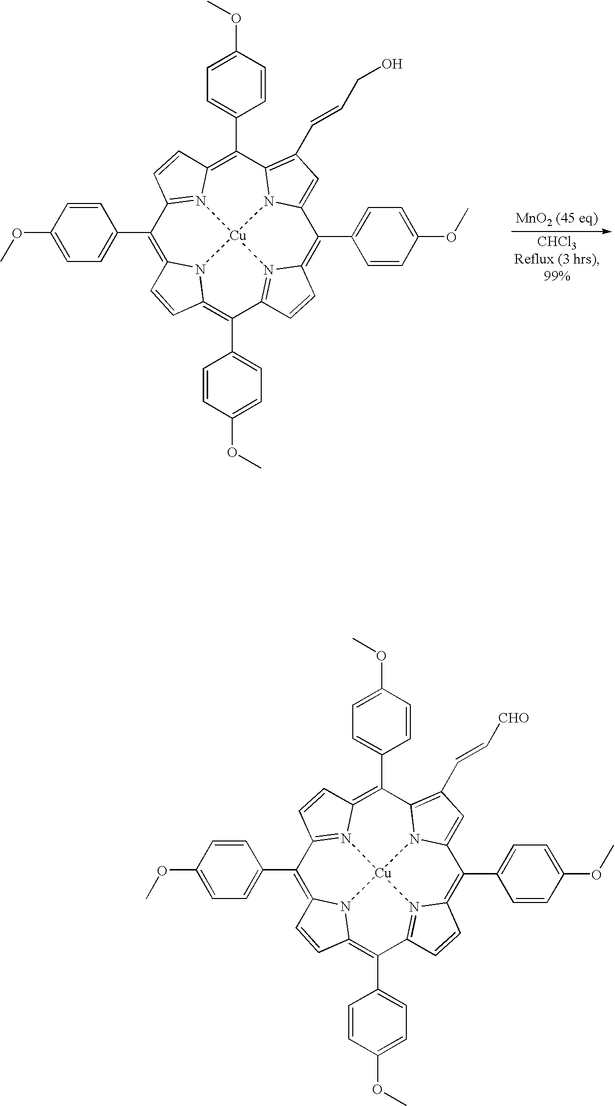

Activated MnO₂ (2.347 g, 27.0 mmol, 45 eq) was added to a solution of CuTMPP-=-CH₂OH (508 mg, 600 μmol) in dry CHCl₃ (30 mL) and heated at reflux temperature for 3 hs under argon atmosphere, TLC analysis (silica, CH₂Cl₂:hexane (2:1)) indicated all staring material had been consumed with the appearance of a single new less polar band. On cooling to room temperature, the solution was filtered through celite and the solvent removed in vacuo. Precipitation from CH₂Cl₂:methanol gave CuTMPP-=-CHO (502 mg, 99%) as a red fine crystals. FAB-LRMS: m/z (%, assignment) cluster at 847-854, 849 (90, M⁺). HRMS: Calcd for M⁺ (C₅₁H₃₈N₄O₅Cu): 849.21382, found: 849.21125.

### CuTMPP-=-=(COOH)₂

### 2-Carboxy-5-(2′-(5′,10′,15′,20′-tetra(4″-methoxylphenyl)porphyrinato copper(II))yl)-penta-2,4-dienoic acid

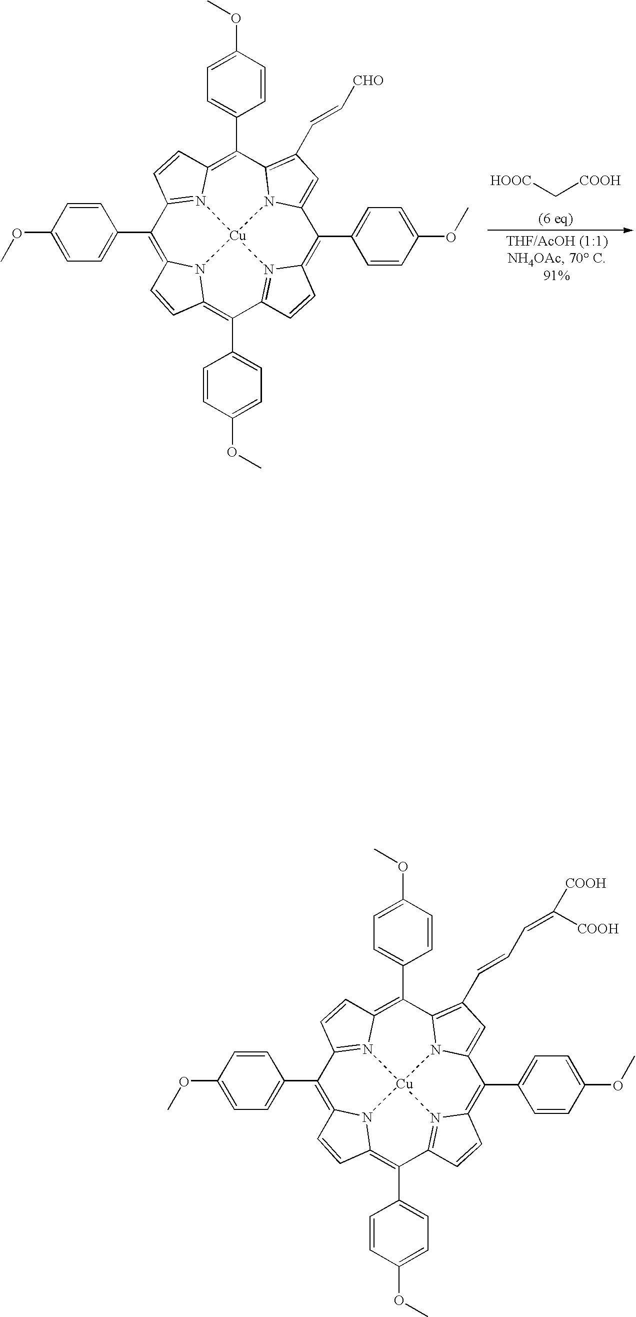

A solution of CuTMPP-=-CHO (494 mg, 580 μmol), malonic acid (363 mg, 3.50 mmol, 6.0 eq) and ammonium acetate (270 mg, 1.20 mmol, 6.0 eq) in a solution of acetic acid:THF (1:2, mL) was heated at 70° C. for 90 min. On cooling to room temperature acetonitrile (50 mL) was added, precipitating the product. CuTMPP-=-=(COOH)₂ (495 mg, 91%) as a dark purple powder was received. FAB-LRMS: m/z (%, assignment) cluster at 1262-1268, 1264 (4, M⁺). HRMS: Calcd for M⁺ (C₈₂H₉₇N₄O₄Zn): 1265.68013, found: 1265.68270.

### ZnTMPP-=-=(COOH)₂

### 2-Carboxy-5-(2′-(5′,10′,15′,20′-tetra(4″-methoxylphenyl)porphyrinato zinc(II))yl)-penta-2,4-dienoic acid

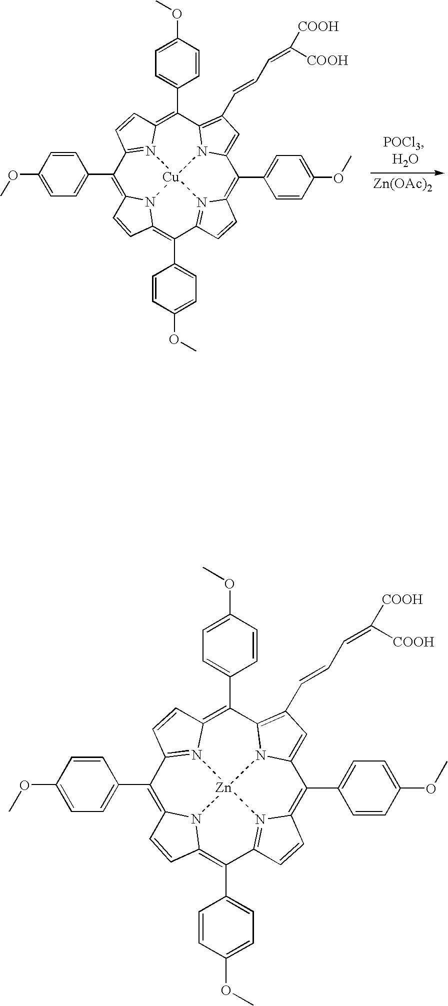

CuTMPP-=-=(COOH)₂ (200 mg, 210 μmol) was dissolved in POCl₃ (20 ccm) and cooled to 0° C. then water (2 mL) was added. The resulting mixture was stirred at 0° C. for 30 min then poured into ice (100 g) and neutralized by ammonia. The dark solid was filtered off. The solid was dissolved in DCM (15 ccm) and zinc acetate dehydrate (84 mg 0.42 mmol) was added in MeOH (1.5 mL). The resulting mixture was stirred for 1 h. The solvents were removed under vacuo and recryst. from DCM/MeOH mixture to give ZnTMPP-=-=(COOH)₂ (150 mg, 77%). FAB-LRMS: m/z (%, assignment) cluster at 932-943, 936 (100, M⁺). HRMS: Calcd for M⁺ (C₅₄H₄₀N₄O₈Zn): 936.21376, found: 936.21142.

Where in the foregoing description reference has been made to integers, elements, features or components having known equivalents, then such equivalents are incorporated as if individually set forth.

Although the invention has been described by way of example and with reference to possible embodiments, it is to be appreciated that improvements and/or modification may be made to the invention without departing from the scope or spirit of the invention.

1. A photoelectric device incorporating a dye-sensitised semiconductor where the bound dye has the structure: 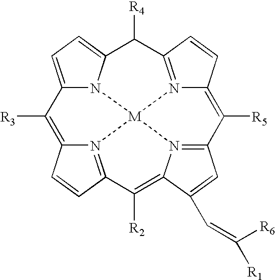
and where:
R₁ is selected from the group consisting of: carboxylic acids, phosphonic acids, sulfonic acids, or salts thereof;
R₂, R₃, R₄ and R₅ are independently selected from the group consisting of: H, substituted or unsubstituted alkyl, substituted or unsubstituted aryl, and substituted or unsubstituted alkyl aryl;
R₆ is selected from the group consisting of: H, CN or —COOH; and
M is absent (and the porphin exists in the free base, protonated diacid, or dianion form) or is selected from the group consisting of: Cu, Ni or Zn.

2. The photoelectric device of claim 1 where the porphin of the dye exists in the metallated form.

3. The photoelectric device of claim 1 where the porphin of the dye is metallated with Zn.

4. The photoelectric device of claim 1 where the semiconductor is selected from the group consisting of: zinc oxide (ZnO), titanium dioxide (TiO₂) and tin dioxide (SnO₂).

5. The photoelectric device of claim 1 where the semiconductor is titanium dioxide (TiO₂).

6. The photoelectric device of claim 1 where the semiconductor is in a mesoporous nanocrystalline form.

7. The photoelectric device of claim 1 where the photoelectric device is a solid state device including a gelled or solid electrolyte or hole transport material.

8. The photoelectric device of claim 1 where R₁ is a carboxylic acid selected from the group consisting of: cyanoacetatic acids, malonatic acids, or salts thereof.

9. The photoelectric device of claim 1 where R₂, R₃, R₄ and R₅ are independently selected from the group consisting of: tert-butyl, phenyl, methylphenyl, methoxyphenyl, ethylphenyl, dimethylphenyl (xylyl), tert-butylphenyl, octylphenyl, di-tert-butylphenyl, and methoxyphenyl.

10. The photoelectric device of claim 1 where R₆ is selected from the group consisting of: H or CN.

11. The photoelectric device of claim 1 where the dye is a cyanoacetic acid and selected from the group consisting of: 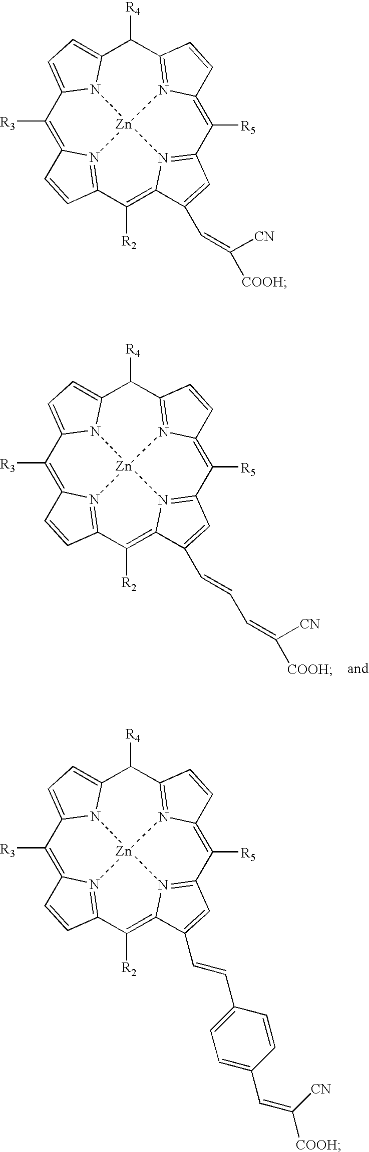
where R₂, R₃, R₄ and R₅ are tert-butyl, phenyl, methylphenyl, ethylphenyl, dimethylphenyl (xylyl), tert-butylphenyl, octylphenyl, di-tert-butylphenyl, or methoxyphenyl.

12. The photoelectric device of claim 1 where the dye is a malonic acid and selected from the group consisting of: 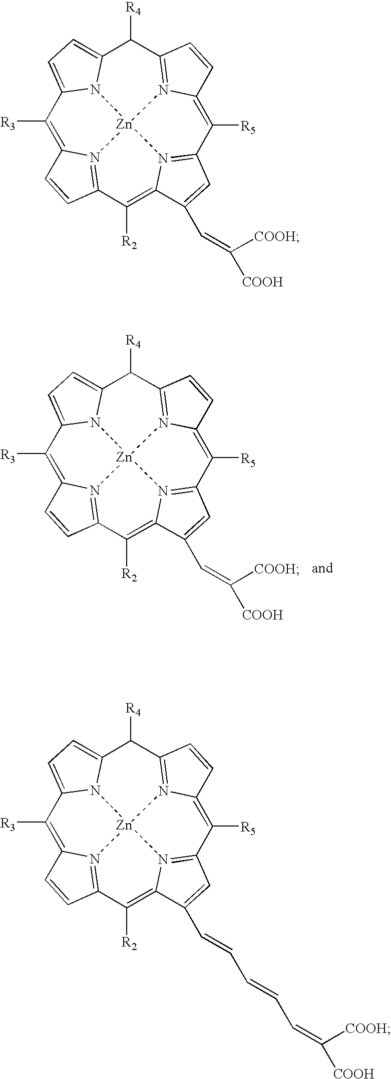
where R₂, R₃, R₄ and R₅ are tert-butyl, phenyl, methylphenyl, ethylphenyl, dimethylphenyl (xylyl), tert-butylphenyl, octylphenyl, di-tert-butylphenyl, or methoxyphenyl.

13. The photoelectric device of claim 1 where the semiconductor includes a surface coating of a non-acceptor.

14. The photoelectric device of claim 1 where the non-acceptor is selected from the group consisting of: 4-tert-butylpyridine and Nb2O₅.

15. The photoelectric device of claim 1 where the electrolyte or hole transport material comprises 2,2′,7,7′-tetrakis(N,N-dip-methoxyphenyl-amine)9,9′-spirobifluorene.

16. The photoelectric device of claim 1 where the electrolyte or hole transport material further comprises tris-(4-bromophenyl)-ammoniumylhexachloroantimonate.

17. The photoelectric device of claim 1 where the electrolyte or hole transport material comprises lithium triflate and tert-butylpyridine.

18. The photoelectric device of claim 1 where the photoelectric device is a photoelectro-chemical cell.

19. The photoelectric device of claim 1 where the photoelectric device is a photoelectro-chemical cell with an overall conversion efficiency of at least 2.5%.

20. The photoelectric device of claim 1 where the photoelectric device is a photoelectro-chemical cell with an overall conversion efficiency of at least 3.0%.

21. A dye for use in the preparation of dye sensitised semiconductors (DSSCs) where the dye has the structure: 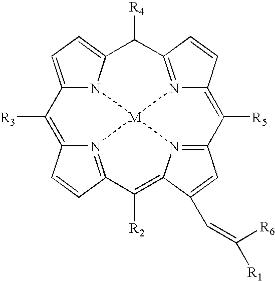
and where:
R₁ is selected from the group consisting of: carboxylates, phosphonates and sulphonates or free acids thereof;
R₂, R₃, R₄ and R₅ are independently selected from the group consisting of: H, substituted or unsubstituted alkyl, substituted or unsubstituted aryl and substituted or unsubstituted alkyl aryl;
R₆ is selected from the group consisting of: H, CN or —COOH; and
M is absent (and the porphin exists in the free base, protonated diacid, or dianion form) or is selected from the group consisting of: Cu, Ni or Zn.

22. The dye of claim 21 where the porphin of the dye exists in the metallated form.

23. The dye of claim 21 where the porphin of the dye is metallated with Zn.

24. The dye of claim 21 where R₁ is a carboxylate selected from the group consisting of:
cyanoacetates, malonates, or free acids thereof.

25. The dye of claim 21 where R₂, R₃, R₄ and R₅ are independently selected from the group consisting of: tert-butyl, phenyl, methylphenyl, methoxyphenyl, ethylphenyl, dimethylphenyl (xylyl), tert-butylphenyl, octylphenyl, di-tert-butylphenyl, and methoxyphenyl.

26. The dye of claim 21 where R₆ is selected from the group consisting of: H or CN.

27. The dye of claim 21 where the dye is a cyanoacetic acid and selected from the group consisting of: 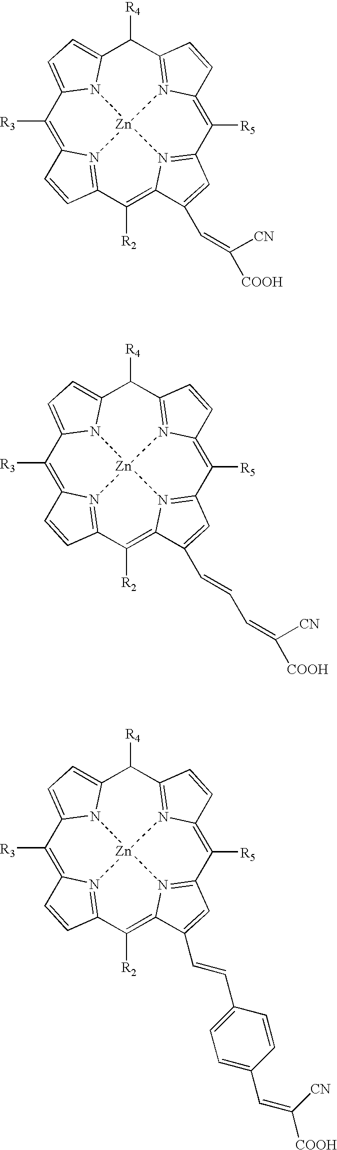
where R₂, R₃, R₄ and R₅ are tert-butyl, phenyl, methylphenyl, ethylphenyl, dimethylphenyl (xylyl), tert-butylphenyl, octylphenyl, di-tert-butylphenyl, or methoxyphenyl.

28. The dye of claim 21 where the dye is a malonic acid and selected from the group consisting of: 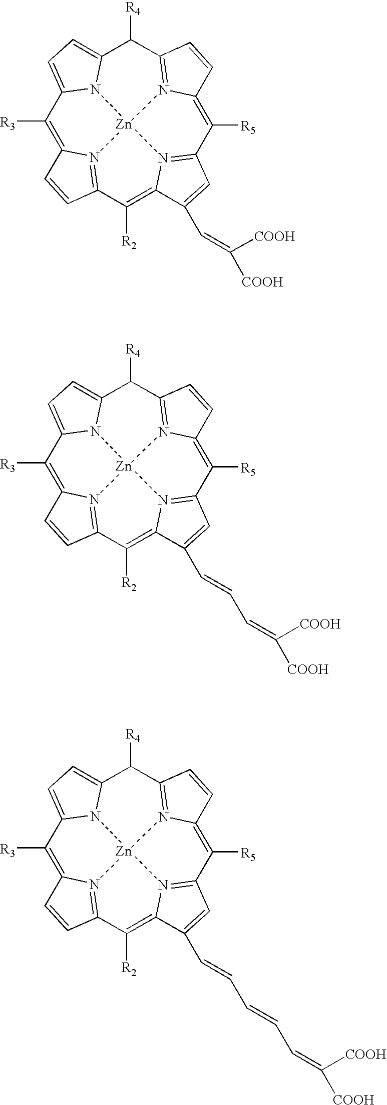
where R₂, R₃, R₄ and R₅ are tert-butyl, phenyl, methylphenyl, ethylphenyl, dimethylphenyl (xylyl), tert-butylphenyl, octylphenyl, di-tert-butylphenyl, or methoxyphenyl.

29. A solid state photovoltaic window comprising nanocrstalline TiO₂ dye sensitised with a dye of claim 21 and an overall conversion efficiency of at least 2.5%.

30. A solid state photovoltaic window comprising nanocrstalline TiO2 dye sensitised with a dye of claim 21 and an overall conversion efficiency of at least 3.0%.
